(19) United States
(12) Patent Application Publication
(10) Pub. No.: US 2024/0112757 A1
(43) Pub. Date: Apr. 4, 2024

(54) METHODS AND SYSTEMS FOR CHARACTERIZING AND TREATING COMBINED HEPATOCELLULAR CHOLANGIOCARCINOMA

(71) Applicant: Foundation Medicine, Inc., Cambridge, MA (US)

(72) Inventors: Karthikeyan MURUGESAN, Boston, MA (US); Siraj ALI, Cambridge, MA (US); Yutong QIU, Pittsburgh, PA (US); Kimberly MCGREGOR, Grand Junction, CO (US)

(73) Assignee: Foundation Medicine, Inc., Cambridge, MA (US)

(21) Appl. No.: 18/274,675

(22) PCT Filed: Jan. 27, 2022
(86) PCT No.: PCT/US2022/014148

§ 371 Date: Jul. 27, 2023

Related U.S. Application Data
(60) Provisional application No. 63/171,423, filed on Apr. 6, 2021.
(60) Provisional application No. 63/143,619, filed on Jan. 29, 2021.

(51) Int. Cl. G16B 40/00; C12Q 1/6886

(52) U.S. Cl. CPC G16B 40/00; C12Q 1/6886

(57) ABSTRACT

A method of characterizing a cancer, such as a combined hepatocellular cholangiocarcinoma (cHCC-CCA), as hepatocellular carcinoma (HCC)-like or cholangiocarcinoma (CCA)-like are described herein, as well as electronic devices and non-transitory computer readable storage mediums for implementing such methods. Also described are methods of treating a cancer, such as cHCC-CCA, characterized as HCC-like or CCA-like. The cancer can be characterized as CCA-like or HCC-like using a cHCC-CCA machine-learning model trained using HCC data from a plurality of HCC samples and CCA data from a plurality of CCA samples. The HCC data, CCA data, and the data from the cancer test sample can include one or more features, such as features from a genomic profile. Exemplary features include tumor purity, a chromosomal aneuploidy status for one or more chromosomes or chromosome arms, and a cancer cell fraction (CCF) for one or more genes differentially represented in CCA and HCC, among others.

### CROSS-REFERENCE TO RELATED APPLICATIONS

This application claims the priority benefit of U.S. Provisional Patent Application Ser. No. 63/143,619, filed on Jan. 29, 2021; and U.S. Provisional Patent Application Ser. No. 63/171,423, filed on Apr. 6, 2021; the content of each of which is incorporated herein by reference.

### FIELD OF THE INVENTION

Described herein are methods, devices, and systems for characterizing a cancer, such as a combined hepatocellular cholangiocarcinoma (cHCC-CCA), as hepatocellular carcinoma (HCC)-like or cholangiocarcinoma (CCA)-like. Also described are methods of treating a cancer, such as cHCC-CCA.

### BACKGROUND

Hepatocellular carcinoma (HCC) and liver cholangiocarcinoma (CCA) are rare, lethal cancers of the liver and have uniquely different treatment strategies. When recent trends demonstrated the rise of CCA in some countries while declining in others, it was thought that this may be due to changes in classification, new diagnostic methods, misclassification rather than an increase or change in lifestyle, or environmental risk factors. Misclassification of tumor type can result in inaccurate prognosis and inefficient disease management and/or treatment. For example, therapeutics vary dramatically between CCA and HCC, as chemotherapeutic agents and more targeted therapies have generally been used to treat CCA, while localized therapies, multi-targeted tyrosine kinase inhibitors, and immunotherapies are generally used to treat HCC.

Combined hepatocellular cholangiocarcinoma (cHCC-CCA) is an even rarer, aggressive primary liver carcinoma, with morphologic features of both HCC and CCA. Histologically, cHCC-CCA can be subdivided into separate, combined, and mixed subtypes on the basis of morphology; however, these classifications have no impact on clinical care. There remains a need to characterize cancers, such as a cHCC-CCA cancer, so that timely and effective treatments can be administered to the patient.

### BRIEF SUMMARY OF THE INVENTION

Described herein are methods for classifying a cancer, such as a combine hepatocellular cholangiocarcinoma (cHCC-CCA) as HCC-like, CCA-like, or ambiguous (e.g., being unable to classify the cancer as HCC-like or CCA-like). The method may be a computer-implemented method, which may be performed, for example, on an electronic system. Also described herein are methods of treating a subject with cancer, which can include obtaining a classification of the cancer in the subject (or a sample, such as a cancer test sample obtained from the subject) as being HCC-like or CCA-like, and treating the cancer using a treatment effective for treating HCC if the cancer is characterized as HCC-like or a treatment effective for treating CCA if the cancer is characterized as CCA-like.

The method for classifying the cancer can include receiving, at one or more processors, test data comprising genomic data (also referred to as “genomic profile data”) associated with a sample from a subject (which may be a cancer sample from the subject, e.g., a sample of the cancer form the subject, for example from a tissue biopsy, or a liquid biopsy sample that includes nucleic acid molecules derived from the cancer); inputting, using the one or more processors, the test data into a combined hepatocellular cholangiocarcinoma (cHCC-CCA) machine-learning model trained using hepatocellular carcinoma (HCC) data comprising HCC genomic data from a plurality of HCC samples and cholangiocarcinoma (CCA) data comprising CCA genomic data from a plurality of CCA samples, wherein the cHCC-CCA machine-learning model is configured to classify sample (or cancer), based on the test data, as CCA-like or HCC-like; and classifying, using the one or more processors and the cHCC-CCA machine-learning model, the sample (or cancer) as HCC-like or CCA-like. In some implementations of the method, the cHCC-CCA machine-learning model may be configured to classify the sample (or cancer), based on the test data, as CCA-like, HCC-like, or ambiguous. The HCC-CCA machine-learning model may be a probabilistic classifier configured to compute a probability that the sample (or cancer) is HCC-like or a probability that the sample (or cancer) is CCA-like. The method can optionally further include training the cHCC-CCA machine-learning model using the HCC data and the CCA data.

The genomic data associated with the sample (i.e., the “test genomic data”) may be generated by sequencing nucleic acid molecules obtained from the sample. For example the genomic data for the sample may be generated by providing a plurality of nucleic acid molecules obtained from the sample from a subject; ligating one or more adapters to one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; and sequencing, by a sequencer (for example, a next generation sequencer), the captured nucleic acid molecules to obtain a plurality of sequence reads that represent the captured nucleic acid molecules, wherein one or more of the plurality of sequencing reads overlap one or more gene loci within a subgenomic interval in the sample. The one or more adapter may comprise amplification primers, flow cell adaptor sequences, substrate adapter sequences, or sample index sequences. The captured nucleic acid molecules may be captured from the amplified nucleic acid molecules by hybridization to one or more bait molecules. For example, the one or more bait molecules may comprise one or more nucleic acid molecules, each comprising a region that is complementary to a region of a captured nucleic acid molecule. Amplifying the nucleic acid molecules may comprise performing a polymerase chain reaction (PCR) amplification technique, a non-PCR amplification technique, or an isothermal amplification technique. The sequencing may comprise use of a massively parallel sequencing (MPS) technique, whole genome sequencing (WGS), whole exome sequencing, targeted sequencing, direct sequencing, or Sanger sequencing technique. In some implementations, the sequencing comprises massively parallel sequencing, and the massively parallel sequencing technique comprises next generation sequencing (NGS).

The cancer characterized or treated according to the methods described herein may be a bile duct cancer. For example, the bile duct cancer could be an intrahepatic bile duct cancer, an extrahepatic bile duct cancer, a perihilar bile duct cancer, or a distal bile duct cancer. In some implementations of the method, the cancer is a cHCC-CCA.

The genomic data for the sample, the HCC genomic data, and the CCA genomic data each include one or more data features. The genomic data for the sample, the HCC genomic data, and the CCA genomic data may each include a tumor purity. The genomic data for the sample, the HCC genomic data, and the CCA genomic data may each include a chromosomal aneuploidy status for one or more chromosomes or chromosome arms. For example, the chromosomal aneuploidy status can include a loss status or a gain status of one or more of a 1 q arm, 2q arm, 5p arm, 6p arm, 6q arm, 7q arm, 8p arm, 8q arm, 10q arm, 17p arm, 17q arm, 18q arm, 20p arm, 20q arm, 21p arm, and 22q arm. The genomic data for the sample, the HCC genomic data, and the CCA genomic data may each include a cancer cell fraction (CCF) for one or more genes, wherein the CCF for the one or more genes is differentially represented in CCA and HCC. For example, the CCF for the one or more genes differentially represented in CCA and HCC may be a CCF of one or more of TP53, CTNNB1, TERT, IDH1, and BAP1. The genomic data for the sample, the HCC genomic data, and the CCA genomic data may each include functional variant status for each of one or more genes (e.g., one or more of ARID1A, BAP1, BRAF, CCND1, CDKN2A, CDKN2B, CTNNB1, ERBB2, FGFR2, IDH1, KRAS, MTAP, PBRM1, PIK3CA, PTEN, MYC, RB1, SMAD4, or TERT, or more particularly one or more of ARID1A, BAP1, CDKN2A, CDKN2B, CTNNB1, FGFR2, IDH1, KRAS, PBRM1, MYC, or TERT). The functional variant status may be, for example, a presence or an absence of the functional variant for the gene. The functional variant may be, for example, a single nucleotide variant (SNV), a multiple nucleotide variant (MNV), a copy number alteration, an indel, or a rearrangement. The genomic data for the sample, the HCC genomic data, and the CCA genomic data may each include a tumor mutational burden (TMB), which may be a continuous numeric feature or a categorical feature. The genomic data for the sample, the HCC genomic data, and the CCA genomic data may each include a microsatellite instability (MSI) status, which may be a numeric feature or a categorical feature. The genomic data for the sample, the HCC genomic data, and the CCA genomic data may each include a genome-wide loss of heterozygosity (gLOH) status, which may be a continuous numeric feature or a categorical feature,

The test data, the HCC data, and the CCA data may each include one or more features that may or may not be genomic features. For example, the test data, the HCC data, and the CCA data may each include an ancestry status. The ancestry status may be a genomic feature, such as a genomic ancestry status. The genomic ancestry status can be a categorical feature, such as a categorical feature, wherein the categorical feature is at least one of African, Ad Mixed American, East Asian, European, or South Asian. The test data, the HCC data, and the CCA data may each include a hepatitis B virus (HBV) status. For example, the HBV status can be determined by detecting a presence or absence of genomic HBV DNA. The test data, the HCC data, and the CCA data may each include one or more clinicopathological features. Exemplary clinicopathological features can include an age of the subject at the time the sample was obtained from the subject, a biological sex of the subject, a sample biopsy site, or a cancer metastasis status.

Genomic features, such as one or more features within the genomic data for the sample, the HCC genomic data, and/or the CCA genomic data may be determined from sequencing data. The sequencing data may be targeted sequencing data, such as targeted sequencing data generated using a hybrid-capture method. The sequencing data may be generated using massively parallel sequencing.

The cHCC-CCA machine-learning model may be a tree-based classification model, for example a tree-based ensemble classification model. The cHCC-CCA machine-learning model may be a bootstrap aggregated model. In some implementations of the method, the model is a random-forest model.

The method of any one of claims 1-35, wherein the cHCC-CCA machine-learning model is a linear classification model. For example, the cHCC-CCA machine-learning model may be a logistic regression model a Naive Bayes classifier, or a support-vector machine model.

The sample may be a solid tissue biopsy sample. For example, the sample may be a formalin-fixed paraffin-embedded (FFPE) sample. Alternatively, the sample can be a liquid biopsy sample comprising circulating tumor DNA (ctDNA). In another alternative, the sample can be a liquid biopsy sample comprising circulating tumor cells (CTCs). In some implementations, the sample is a liquid biopsy sample and comprises blood, plasma, cerebrospinal fluid, sputum, stool, urine, or saliva.

The method may further include generating a report identifying the sample (or cancer) as HCC-like or CCA-like. The report may be displayed, for example on an electronic display. The report may be transmitted to another party, such as the subject or a healthcare provider for the subject. For example, the report may be an electronic medical record, which can be transmitted (e.g., via a computer network or peer-to-peer connection) to the subject or a healthcare provider for the subject.

The method may further include obtaining the sample from the subject.

A method of selecting a treatment for a cancer in a subject can include: obtaining a classification of a cancer or a sample associated with the cancer as HCC-like or CCA-like, wherein the cancer or sample is classified using any of the above methods; and selecting the treatment for the cancer, wherein the treatment is selected to effectively treat HCC if the cancer is classified as HCC-like, and the treatment is selected to effectively treat CCA if the cancer is classified as CCA-like.

A method for treating a cancer in a subject can include obtaining a classification of a cancer or sample from the subject as HCC-like or CCA-like using any of the above methods; and administering a treatment to the subject, wherein the treatment is selected to effectively treat HCC if the cancer is classified as HCC-like, and the treatment is selected to effectively treat CCA if the cancer is classified as CCA-like.

If the cancer is classified as HCC-like, the treatment may include, for example, a localized therapy, a multi-targeted tyrosine kinase inhibitor, or an immunotherapy. In some implementations, the treatment includes a multi-targeted tyrosine kinase inhibitor. For example, the multi-targeted tyrosine kinase inhibitor may be axitinib, brivanib, cabozantinib, cediranib, donofenib, dovitinib, lenvatinib, linifanib, nintedanib, regorafenib, sorafenib, or sunitinib. In some implementations, the treatment includes an immunotherapy. For example, the immunotherapy may be an immune checkpoint inhibitor. Exemplary immune checkpoint inhibitors include tremelimumab, ipilimumab, nivolumab, pembrolizumab, camrelizumab, tislelizumab, avelumab, atezolizumab, or durvalumab.

If the cancer is classified as HCC-like, the treatment may include, for example, chemotherapy or a targeted therapy. In some implementations, the treatment includes a chemotherapy. Exemplary chemotherapies can include the administration of a fluoropyrimidine, a platinum agent, or a taxane. For example, the chemotherapy may include gemcitabine, capecitabine, doxifluridine, fluorouracil, irinotecan, tegafur, cisplatin, oxaliplatin, docetaxel, or paclitaxel. In some implementations, the treatment includes a targeted therapy. For example, the targeted therapy may include a kinase-specific inhibitor. The HCC-like treatment may include administration of an IDH1 inhibitor, an FGFR2 inhibitor, a MEK inhibitor, or an mTOR inhibitor. For example, the treatment may include administration of an IDH1 inhibitor (for example, ivosidenib), for example when the cancer has an IDH1 mutation. The treatment may include administration of an FGFR2 inhibitor (for example, pemigatinib, infigratinib, derazantinib, or bemarituzumab), for example when the cancer has an FGFR2 mutation. The treatment may include administration of a MEK inhibitor (such as selumetinib) or an mTOR inhibitor (such as everolimus), for example when the cancer has a KRAS mutation.

Also described herein is a system, which includes one or more processors; a memory; and one or more programs, wherein the one or more programs are stored in the memory and configured to be executed by the one or more processors, the one or more programs including instructions for implementing the any of the above methods. The system optionally includes a sequencer configured to sequence nucleic acids derived from sample.

Further described herein is a non-transitory computer-readable storage medium storing one or more programs, the one or more programs comprising instructions, which when executed by one or more processors of an electronic device, cause the electronic device to implement the method of any one of the above methods.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows relative feature importance of an exemplary set of data features used to train an exemplary cHCC-CCA machine-learning model (the top 50 of 157 features are shown), in some embodiments.

FIG. 2 shows an exemplary method for training and operating the cHCC-CCA machine-learning model configured to classify a cHCC-CCA cancer as HCC-like or CCA-like.

FIG. 3 is a flowchart of an exemplary computer-implemented method of characterizing a cHCC-CCA, which may be performed at an electronic device.

FIG. 4 shows an example of a computing device in accordance with one embodiment, which may be used with the methods described herein.

FIG. 5 shows a comparison of the TMB distribution across the CCA, cHCC-CCA, and HCC samples according to an exemplary embodiment.

FIG. 6 shows a comparison of the gLOH distribution across the CCA, cHCC-CCA, and HCC samples according to an exemplary embodiment.

FIG. 7 shows a volcano plot depicting the co-occurrence and mutual exclusivity of aneuploidy events between CCA and HCC according to an exemplary embodiment.

Chromosomal arm aneuploidies with a log 10 odds ratio greater than 0 are associated with CCA, and chromosomal arm aneuploidies having a log 10 odds ratio lower than 0 are associated with HCC. Only aneuploidy events with an adjusted P value≤0.01 and a prevalence≥10% in at least one disease are labelled. The two-tailed Fisher's exact test was used to evaluate the P values and odds ratios, which is used to determine associations between an event and disease. The Benjamini-Hochberg procedure was used to estimate the adjusted P values.

FIG. 8 shows a landscape of the chromosomal aneuploidies detected according to an exemplary embodiment across the cHCC-CCA samples, with the X-axis representing each cHCC-CCA sample and the Y-axis representing the assessed aneuploidy events.

FIG. 9 shows a volcano plot depicting the co-occurrence and mutual exclusivity of gene alterations between CCA and HCC according to an exemplary embodiment. Only genes with an adjusted P value≤0.05 and a prevalence ≥5% in either disease are labelled. A two-tailed Fisher's exact test was used to evaluate the P values and odds ratios that determines associations between genes and disease. The Benjamini-Hochberg procedure was used to estimate the adjusted P values. Genes with a log 10 odds ratio greater than 0 are associated with CCA, and genes having a log 10 odds ratio lower than 0 are associated with HCC.

FIG. 10 shows the prevalence of functional variants in select genes among the CCA, cHCC-CCA, and HCC samples, according to an exemplary embodiment. For each gene, CCA, cHCC-CCA, and HCC are shown from left to right.

FIG. 11 compares the computational tumor purity across CCA, HCC, and cHCC-CCA samples, according to an exemplary embodiment. The p values were estimated using a Wilcoxon rank sum test, with **** denoting a p-value<0.0001.

FIG. 12A shows 10-fold cross-validation metrics (AUC, log loss, precision, sensitivity, and specificity) for an example trained cHCC-CCA machine-learning model that used only genomic-based features, according to an exemplary embodiment.

FIG. 12B shows 10-fold cross-validation metrics (AUC, log loss, precision, sensitivity, and specificity) for an example trained cHCC-CCA machine learning model that used genomic-based features and clinicopathological features, according to an exemplary embodiment.

FIG. 13A shows an AUC (ROC) curve for HCC test samples and CCA test samples characterized using an example trained cHCC-CCA machine-learning model trained using only genomic-based features from labeled HCC samples and CCA samples, according to an exemplary embodiment.

FIG. 13B shows an AUC (ROC) curve for HCC test samples and CCA test samples characterized using an example trained cHCC-CCA machine-learning model trained using genomic-based features and clinicopathological features from labeled HCC samples and CCA samples, according to an exemplary embodiment.

FIG. 14 shows the prevalence of a functional variant in certain genes in CCA, model-classified CCA-like cHCC-CCA, model-classified HCC-like cHCC-CCA, and HCC, according to an exemplary embodiment.

FIG. 15 shows the median cancer cell fraction (CCF) for targeted genes for CCA samples, HCC samples, cHCC-CCA samples classified as CCA-like using an exemplary trained cHCC-CCA machine-learning model, cHCC-CCA samples classified as HCC-like using an exemplary trained cHCC-CCA machine-learning model, and cHCC-CCA samples classified as ambiguous using an exemplary trained cHCC-CCA machine-learning model, according to an exemplary embodiment.

FIG. 16A shows the median cancer cell fraction (CCF) for TP53 for CCA samples, HCC samples, cHCC-CCA samples classified as CCA-like using an exemplary trained cHCC-CCA machine-learning model, cHCC-CCA samples classified as HCC-like using an exemplary trained cHCC-CCA machine-learning model, and cHCC-CCA samples classified as ambiguous using an exemplary trained cHCC-CCA machine-learning model, according to an exemplary embodiment.

FIG. 16B shows the median cancer cell fraction (CCF) for CTNNB1 and TERT for CCA samples, HCC samples, cHCC-CCA samples classified as CCA-like using an exemplary trained cHCC-CCA machine-learning model, cHCC-CCA samples classified as HCC-like using an exemplary trained cHCC-CCA machine-learning model, and cHCC-CCA samples classified as ambiguous using an exemplary trained cHCC-CCA machine-learning model, according to an exemplary embodiment.

FIG. 16C shows the median cancer cell fraction (CCF) for IDH1 and BAP1 for CCA samples, HCC samples, cHCC-CCA samples classified as CCA-like using an exemplary trained cHCC-CCA machine-learning model, cHCC-CCA samples classified as HCC-like using an exemplary trained cHCC-CCA machine-learning model, and cHCC-CCA samples classified as ambiguous using an exemplary trained cHCC-CCA machine-learning model, according to an exemplary embodiment.

FIG. 17 shows the lack of correlation between cancer cell fraction (CCF) and tumor purity in CCA and HCC samples.

FIG. 18 shows a histogram of the random forest-based prediction probabilities for 73 cHCC-CCA cases, according to an exemplary method. The HCC prediction probability of the cHCC-CCA cases is depicted in the histogram and the disease prediction of ambiguous, CCA-like and HCC-like based on the probability threshold (0.61 here) that maximized the Matthew's correlation coefficient in the HCC-CCA training cohort, is overlayed.

### DETAILED DESCRIPTION OF THE INVENTION

Described herein are methods of classifying a cancer, such as a combined hepatocellular cholangiocarcinoma (cHCC-CCA), as HCC-like or CCA-like. Current techniques for characterizing cHCC-CCA are often insufficient for making treatment decisions, leading healthcare provider uncertain as to how the patient should be treated. As further described herein, various data features, including genomic data, associated with the cancer have been identified that indicate whether the cHCC-CCA cancer is more HCC-like or more CCA-like, which indicates how the cHCC-CCA cancer should be treated. A machine-learning model trained using HCC data, including HCC genomic data and, CCA data, including CCA genomic data, can be used to classify the test cHCC-CCA cancer as HCC-like or CCA-like.

Certain data features associated with the cHCC-CCA have been identified as being particularly useful for characterizing the cHCC-CCA as HCC-like or CCA-like. For example, tumor purity of the sample obtained from the subject is a particularly useful distinguishing factor. Aneuploidy status for one or more chromosomes or chromosome arms was also discovered to be useful distinguish feature. Other useful distinguishing features that have been identified are described herein, including functional variant status (e.g., the presence or absence of a functional variant) of select genes, tumor mutational burden (TMB), microsatellite instability (MSI) status, genome-wide loss of heterozygosity (gLOH) status, genetic ancestry status, and hepatitis B virus (HBV) status. Although these have been identified as useful indicators, it is not necessary to include all or even a majority of the data features. Additional or fewer data features may be selected to obtain the desired performance of the characterization method.

Once the cHCC-CCA in the subject has been characterized, it may be treated in a manner that depends on whether the cHCC-CCA has been characterized as HCC-like or CCA-like. For example, a treatment configured to treat HCC (such as a local therapy, a multi-targeted tyrosine kinase inhibitor, or an immunotherapy) may be administered to the subject if the cHCC-CCA is characterized as HCC-like, and a treatment configured to treat CCA (such as chemotherapy or a targeted therapy) may be administered to the subject if the cHCC-CCA is characterized as CCA-like.

While reference is made to a combined hepatocellular cholangiocarcinoma (cHCC-CCA), and classifying the cancer as HCC-like or CCA-like, it will be understood that methods are not limited thereto. For example, the cancer may be a combination of two or more cancers (e.g., a first type of carcinoma and a second type of carcinoma), and the cancer may be classified as first-carcinoma-like or second-carcinoma-like. In some instances, classification may not be possible based on the combined cancer type, and the classification may be ambiguous (e.g., neither first-carcinoma-like nor second-carcinoma-like). It will also be understood that that the cancer may include a combination of three, four, five, or more carcinomas, and classification may include classifying the cancer based on all of the combinations of carcinomas. For example, the method may include receiving, at one or more processors, test data for a sample from a subject with cancer, wherein the test data comprises genomic data for the sample; inputting, using the at least one processor, the test data into a machine-learning model trained using a first carcinoma data comprising a first carcinoma genomic data from a plurality of first type of carcinoma samples and a second carcinoma data comprising second carcinoma genomic data from a plurality of second type of carcinoma samples, wherein the first carcinoma samples are different from the second carcinoma samples, and wherein the machine-learning model is configured to classify the sample, based on the test data, as first-carcinoma-like, second-carcinoma-like, or ambiguous; and classifying, by the at least one processor using the machine-learning model, the sample as first-carcinoma-like, second-carcinoma-like, or ambiguous.

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

### Definitions

As used herein, the singular forms “a,” “an,” and “the” include the plural reference unless the context clearly dictates otherwise.

Any reference to “or” herein is intended to encompass “and/or” unless otherwise stated.

Reference to “about” a value or parameter herein includes (and describes) variations that are directed to that value or parameter per se. For example, description referring to “about X” includes description of “X”.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

The terms “individual,” “patient,” and “subject” are used synonymously, and refer to a mammal, and includes, but is not limited to, human, bovine, horse, feline, canine, rodent, or primate. In one embodiment, the subject is a human.

The term “effective amount” used herein refers to an amount of a compound or composition sufficient to treat a specified disorder, condition or disease, such as ameliorate, palliate, lessen, and/or delay one or more of its symptoms. In reference to a cancer, an effective amount comprises an amount sufficient to cause the number of cancer cells present in a subject to decrease in number and/or size and/or to slow the growth rate of the cancer cells. In some embodiments, an effective amount is an amount sufficient to prevent or delay recurrence of the disease. In the case of cancer, the effective amount of the compound or composition may: (i) reduce the number of cancer cells; (ii) inhibit, retard, slow to some extent and preferably stop cancer cell proliferation; (iii) prevent or delay occurrence and/or recurrence of the cancer; and/or (iv) relieve to some extent one or more of the symptoms associated with the cancer.

As used herein, the term “subgenomic interval” (or “subgenomic sequence interval”) refers to a portion of a genomic sequence.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In reference to a cancer, the number of cancer cells present in a subject may decrease in number and/or size and/or the growth rate of the cancer cells may slow. In some embodiments, treatment may prevent or delay recurrence of the disease. In the case of cancer, the treatment may: (i) reduce the number of cancer cells; (ii) inhibit, retard, slow to some extent and preferably stop cancer cell proliferation; (iii) prevent or delay occurrence and/or recurrence of the cancer; and/or (iv) relieve to some extent one or more of the symptoms associated with the cancer. The methods of the invention contemplate any one or more of these aspects of treatment.

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Data Features

Test data associated with the test sample for the subject with the cHCC-CCA being characterized includes one or more data features that can be used as an input for the cHCC-CCA machine-learning model. The cHCC-CCA machine-learning model is also trained based on corresponding data features for example from HCC data associated with HCC training samples and CCA data associated with CCA training samples. Thus, a description of a data feature for the test data similarly applies to the HCC data and the CCA data. The machine learning model may be trained using more data features than used as input (i.e., that within the test data), for example by adjusting a weight for data features omitted from the test data.

The data features (i.e., the test data, the HCC data, and/or CCA data) can include genomic data for the sample. The genomic data includes genomic information from the sample, which may be obtained, for example, by sequencing genomic DNA or RNA (e.g., mRNA or miRNA) from the sample to obtain sequencing data. The sequencing data may be targeted sequencing data, such as sequencing data generated using a hybrid-capture method. See, e.g., WO 2012/092426 A1. Sequencing data may alternatively or additionally be whole genome sequencing (WGS) data, whole exome sequencing (WES) data, or RNA sequencing (RNA-seq) data. Sequencing data may be obtained, for example, using a next-generation sequencing method (also referred to as massively parallel sequencing). The sequencing data can be analyzed using known methods to derive the genomic data.

Nucleic acid molecules (e.g., DNA or RNA, such as mRNA or miRNA) may be derived from a solid tissue sample (i.e., a solid tissue biopsy). The tissue sample may be fresh, frozen, or preserved, such as a formalin-fixed, paraffin-embedded (FFPE) tissue sample. For example, nucleic acids may be derived from a cHCC-CCA tissue biopsy from the subject, which can be analyzed (e.g., by sequencing the nucleic acid molecules) to determine the genomic data for the sample associated with the test sample. Alternatively, the nucleic acid molecules may be derived from a liquid sample from the patient (i.e., a liquid biopsy) that include circulating tumor DNA (ctDNA). The liquid sample may be, for example, blood, serum, cerebrospinal fluid, sputum, stool, urine, saliva, or other liquid containing ctDNA. HCC genomic data and CCA genomic data may be obtained, for example to train the cHCC-CCA machine-learning algorithm, by deriving nucleic acid molecules from known HCC samples and known CCA samples, respectively.

Tumor purity of the tissue sample was found to be a significant feature in determining whether the cHCC-CCA sample is more likely to be HCC-like or CCA-like. Thus, the feature data (i.e., the test data, as well as the HCC data and the CCA data used to train the cHCC-CCA machine-learning model) can include a tumor purity parameter. The tumor purity parameter indicates what portion of the tissue sample is tumorous tissue, as the biopsy sample can include a mixture of tumor cells and healthy tissue cells (e.g., tumor-associated stromal cells, tumor infiltrating leukocytes, etc.). The tumor purity may be computationally determined or manually determined. This parameter can be computationally determined, for example, from the sequencing data, which may be analyzed to determine the fraction of tumor-associated nucleic acids in the sample. The feature may be determined as a statistical quantification of the amount of tumor nucleic acids. For example, the tumor purity parameter may be derived by simultaneously fitting segments of genomic allele counts and corresponding SNP frequencies to various statistical models, of which tumor purity is a modeling parameter. Exemplary methods for determining tumor purity include methods described in Su et al., PurityEst: estimating purity of human tumor samples using next-generation sequencing data, Bioinformatics, vol. 28, no. 17, pp. 2265-2266 (2012) or Sun et al., A computational approach to distinguish somatic vs. germline origin of genomic alterations from deep sequencing of cancer specimens without a matched normal, PLoS Computational. Biology, vol. 14, no. 2, e1005965 (2018). The tumor purity can alternatively be manually determined, for example by microscopy. For example, the sample may be observed under a microscope, and the percentage of cancer cells in the sample can be determined.

Chromosomal aneuploidy status for one or more chromosomes or chromosome arms may be included in the genomic data (i.e., for the test, CCA, and HCC samples) for the cHCC-CCA machine-learning model. The chromosomal aneuploidy status may be a categorical feature. For example, the chromosomal aneuploidy status of any given chromosome or chromosomal arm may be a binary feature indicating the gain or no gain (or loss or no loss) of the chromosome or chromosomal arm. Alternatively, the chromosomal aneuploidy status may be a numerical feature. For example, the chromosomal aneuploidy status may be a fraction gain or fraction loss of the chromosome or chromosomal arm. The gain or loss may be indicated in separate features (i.e., a first feature inciting the presence or absence, or fraction, of the chromosome or chromosomal arm gain, and a second feature indicating the presence or absence, or fraction, of the chromosome or chromosomal arm loss). Alternatively, the gain or loss may be indicated as a combined feature (for example, a three-part categorical feature that indicates chromosome or chromosomal arm loss, gain, or wild type). Chromosomal aneuploidy status of any give chromosome or chromosomal arm can be determined using sequencing read counts from the sequencing data. For example, a chromosomal aneuploidy status of a given chromosome or chromosomal arm can be determined by comparing a log ratio of read counts attributed to the cancerous cells (i.e., the cHCC-CCA of the subject, or the HCC from the HCC training sample or the CCA from the CCA training sample) to a process matched normal control. Signal and noise metrics can be determined to measure chromosome or chromosomal arm copy number, and, based on the noise metric of each sample, a per sample limit of detection can be calculated. The chromosomal aneuploidy status can be determined using methods for copy number calling, for example the methods described in Frampton et al., Development and validation of a clinical cancer genomic profiling test based on massively parallel DNA sequencing, Nature Biotechnology, vol. 31, no. 11, p. 1023-1031 (2013) or Sun et al., A computational approach to distinguish somatic vs. germline origin of genomic alterations from deep sequencing of cancer specimens without a matched normal, PLoS Computational. Biology, vol. 14, no. 2, e1005965 (2018), except that the copy number call is made in reference to the full chromosome or a chromosomal arm, or a fraction thereof. The chromosome or chromosomal arm can be considered lost or gained if more than a predetermined threshold of the chromosome or chromosomal arm is lost or gained. The predetermine threshold may be, for example, about 30% or higher, about 40% or higher, about 50% or higher, about 60% or higher, or about 70% or higher. In some embodiments, the predetermined threshold is 50% or higher. In some embodiments, the predetermined threshold is 50%.

In some embodiments, the chromosome or chromosomal arm loss is included as a feature. In some embodiments, the chromosome or chromosomal arm gain is included as a feature. In some embodiments, both the chromosome or chromosomal arm gain and the chromosome or chromosomal arm loss are included as features. 1q arm, 2q arm, 5p arm, 6p arm, 6q arm, 7q arm, 8p arm, 8q arm, 10q arm, 17p arm, 17q arm, 18q arm, 20p arm, 20q arm, 21p arm, and 22q arm have been identified as being useful for distinguishing CCA and HCC, and a chromosomal aneuploidy status of one or more of these chromosomal arms may be included in the genomic data for the test, HCC and/or CCA sample for the cHCC-CCA machine learning model. In some embodiments, the chromosomal aneuploidy status of the genomic data comprises a chromosomal aneuploidy status of one or more of a 3p chromosomal arm loss, a 9q chromosomal arm loss, a 9p chromosomal arm loss, a 6q chromosomal arm loss, a 1q chromosomal arm gain, a 14q chromosomal arm loss, a 12q chromosomal arm loss, a 6p chromosomal arm gain, a 8p chromosomal arm loss, a 8q chromosomal arm gain, a 17p chromosomal arm loss, a 5q chromosomal arm gain, a 16q chromosomal arm loss, a 18q chromosomal arm loss, a 16p chromosomal arm loss, a 13q chromosomal arm loss, a 4q chromosomal arm loss, a 12p chromosomal arm loss, a 2q chromosomal arm gain, a 22q chromosomal arm loss, a 3q chromosomal arm gain, and a 1p chromosomal arm loss. In some embodiments, the chromosomal aneuploidy status of the genomic data comprises a chromosomal aneuploidy status of a 3p chromosomal arm loss. In some embodiments, the chromosomal aneuploidy status of the genomic data comprises a chromosomal aneuploidy status of a 3p chromosomal arm loss and a 9q chromosomal arm loss. In some embodiments, the chromosomal aneuploidy status of the genomic data comprises a chromosomal aneuploidy status of a 3p chromosomal arm loss, a 9q chromosomal arm loss, and a 9p chromosomal arm loss.

The genomic data can include a cancer cell fraction (CCF) of one or more genes that can distinguish CCA and HCC. The CCF of certain genes can differ between CCA and HCC cancers. Thus, the CCF for a particular gene may be higher for an HCC population than a CCA population (i.e., an HCC-associated gene), or the CCF for a particular gene may be higher for a CCA population than an HCC population (i.e., a CCA-associated gene). That is, the CCF for the gene is differentially represented in CCA and HCC. Certain genes can therefore can be used as a marker for characterizing cHCC-CCA as HCC-like or CCA-like due to the CCF differential between CCA and HCC. For example, gene alterations in IDH1 are more clonal in CCA than HCC and gene alterations in TERT are more clonal in HCC than CCA, even though the CCF across all short variants in HCC and CCA may be similar. Exemplary genes associated with CCF differential between HCC and CCA include BAP1, CTNNB1, IDH1, TERT, and TP53. Thus, in some embodiments, the genomic data can include a cancer cell fraction (CCF) of one or more genes for which the CCF statistically differentiates CCA and HCC. In some embodiments, the genomic data a cancer cell fraction (CCF) of one or more (or two or more, or three or more, or four or more, or all) of BAP1, CTNNB1, IDH1, TERT, and TP53. In some embodiments, the genomic data a cancer cell fraction (CCF) of TERT. In some embodiments, the genomic data a cancer cell fraction (CCF) of TERT and IDH1. In some embodiments, the genomic data a cancer cell fraction (CCF) of BAP1, IDH1, TERT, and TP53. In some embodiments, the genomic data a cancer cell fraction (CCF) of BAP1, CTNNB1, IDH1, TERT, and TP53. An exemplary method of determining CCF is PyClone, generally described in Roth et al., PyClone: statistical inference of clonal population structure in cancer, Nat Methods, vol. 11, pp 396-398 (2014).

The functional variant status of one or more genes may be included in the genomic data. The functional variant is a variant that alters the function of the gene product, for example by upregulating or downregulating expression or activity of the gene product, or a variant that is associated with pathogenicity. The functional variant status may be included, for example, as a binary feature indicating the presence or absence of any functional variant in the gene or the presence or absence of any functional variant in the gene caused by particular alteration type (e.g., a short variant (such as a single nucleotide variant (SNV), a multiple nucleotide variant (MNV), an indel, a missense mutation, a nonsense small indel, a frameshift mutation, a non-frameshift mutation, a splice site mutation, or a promotor-associated mutation), a copy number alteration (e.g., an amplification or a deletion), or a rearrangement (e.g., a fusion, a truncation, a duplication, or an inversion), or the presence or absence of a particular functional variant in the gene (for example, a presence or absence of a specific mutation (e.g., EGFR L858R). The functional variant may be, for example, a variant from an annotated database indicating the variant as a functional variant, such as the COSMIC database (see Forbes et al., COSMIC: Somatic cancer genetics at high-resolution, Nucleic Acids Research, vol. 45, no. D1, pp. D777-D783 (2017)) or may be a frameshif or truncation variant. Variants of unknown significance can optionally be excluded from the functional variants. The one or more genes included for the functional variant status data feature are genes that can differentiate HCC and CCA, such as one or more of ARID1A, BAP1, BRAF, CCND1, CDKN2A, CDKN2B, CTNNB1, ERBB2, FGFR2, IDH1, KRAS, MTAP, PBRM1, PIK3CA, PTEN, MYC, RB1, SMAD4, or TERT. In some embodiments, the one or more genes comprises ARID1A, BAP1, CDKN2A, CDKN2B, CTNNB1, FGFR2, IDH1, KRAS, PBRM1, MYC, or TERT.

Tumor mutational burden (TMB) can be included in the genomic data. TMB can be measured as a number of mutations in the nucleic acids from cancer cells per amount of the genome sequenced (e.g., number of mutations per 1 megabase (Mb) of genome sequence, or per 10 Mb of genome sequenced). In some embodiments, the TMB is encoded as a continuous numeric feature. In some embodiments, the TMB is encoded as a categorical feature, for example if the TMB is above or below a predetermined threshold (e.g., a predetermined threshold set at about 1 mutations/Mb or higher, about 5 mutations/Mb or higher, about 10 mutations/Mb or higher, about 15 mutations/Mb, or about 20 mutations/Mb or higher).

The genomic data may include a microsatellite instability (MSI) status. The MSI status may be included as a categorical feature. For example, the MSI status may be categorized as MSI-high (MSI-H), MSI-intermediate (MSI-I), or MSI-stable (MSS). Optionally, MSI-low (MSI-L) may be included in addition to or in the alternative from MSI-I. Optionally, an MSI-unknown (MSI-U) may be included if the MSI status of the cancer is unknown. Alternatively, the MSI status may be considered as a binary feature, for example MSI-H or not, or MSS-S or not. See, for example, Trabucco et al., A Novel Next-Generation Sequencing Approach to Detecting Microsatellite Instability and Pan-Tumor Characterization of 1000Microsatellite Instability-High Cases in 67,000 Patient Samples, J. Molecular Diagnostics, vol. 21, no. 6, pp. 1053-1066 (2019), which provides an exemplary method of categorizing MSI-H, MSI-I and MSS cancers.

Genomic loss of heterozygosity (gLOH) (e.g., a genome-wide loss of heterozygosity or exome-wide loss of heterozygosity) may optionally be included as a data feature within the genomic data. The full genome need not be analyzed to determine the genomic loss of heterozygosity, as whole exome sequencing or targeted sequencing across a large enough portion of the genome may be taken as a proxy from genomic loss of heterozygosity. In some embodiments, the gLOH is encoded as a continuous numeric feature. In some embodiments, the gLOH is encoded as a categorical feature, for example if the gLOH is above or below a predetermined threshold. The predetermined threshold may be set, for example, at about 10% or higher, about 12% or higher, about 14% or higher, or about 16% or higher). The predetermined threshold may be set, for example, at about 16%. The gLOH may be determined, for example, using the methods described in Swisher et al., Rucaparib in relapsed, platinum-sensitive high-grade ovarian carcinoma (ARIEL2 Part1): an international, multicenter, open-label, phase 2 trial, Lancet Oncology, vol. 18, no. 1, pp. 75-87 (2017).

The data features used to characterize the cHCC-CCA as CCA-like or HCC-like using the cHCC-CCA machine-learning model can include an ancestry status. The ancestry status may be, for example, a self-reported ancestry status or a genomic ancestry status. The genomic ancestry status may be part of the of the genomic data. The genomic ancestry may be based on, for example, variants (e.g., SNPs), methylation status, gene expression, miRNA sequences or expression, or other features. The genomic ancestry status can be used as a categorical feature. Exemplary categorical annotations can include African, Ad Mixed American, East Asian, European, and South Asian. See, for example, Newberg et al., Abstract 1599: Determining patient ancestry based on targeted tumor comprehensive genomic profiling, Cancer Reesarch, vol. 79, no 13 Supplement (2019), and Carrot-Zhang, Comprehensive Analysis of Genetic Ancestry and its Molecular Correlates in Cancer, Cancer Cell, vol. 37, no. 5, pp. 639-654 (2020).

The data features (i.e., for the test data, as well as the CCA data and the HCC data) may include a hepatitis B virus (HBV status). The HBV status can be a categorical feature, with the subject either being HBV-positive or HBV-negative. The HBV status can be determined using sequencing data, by identifying sequencing reads associated with genomic HBV DNA. For example, sequencing reads that do not map to a human reference genome can be assembled into contigs, and the contigs can be queried to determine the presence or absence of HBV. Alternatively or additionally, the HBV status may be determined using a serological test, such as a test for antibodies to HBV.

The data features may optionally include an anatomic subclassification of the cancer in the subject. For example, the anatomic subclassification may indicate that the cancer is an intrahepatic tumor, a perihilar tumor, or an extrahepatic tumor.

Other clinicopathologoical features of the subject or the cancer may also be used as data features to characterize the cHCC-CCA as HCC-like or CCA-like. Exemplary clincopatological features can include, but are not limited to, an age of the subject at the time the test sample was obtained from the subject, a biological sex of the subject, a test sample biopsy site, a cancer metastasis status, stage of disease, hepatitis C virus status, smoking status, alcohol consumption, diabetes status, obesity status (or body-mass index), encephalopathy status, ascites status, serum albumin level, serum bilirubin level, estrogen levels, or vitamin levels. In some embodiments, the clincopatological features include one or more of an age of the subject at the time the test sample was obtained from the subject, a biological sex of the subject, a test sample biopsy site, or a cancer metastasis status (e.g., local, metastatic, or lymph node). The test sample biopsy site is the location within the subject that the test sample is biopsied, for example in the event of a metastatic cHCC-CCA the tumor may be biopsied at a location other than the location of the primary tumor. Exemplary test sample biopsy sites can include a soft tissue, a liver, bone, omentum, kidney, chest wall, adrenal gland, or brain, among other locations in the subject.

Other data features may be used in the cHCC-CCA characterization method. For example, the features may include on or more of a methylation signature, an mRNA expression level, an miRNA expression level, a proteomics feature, or an immunohistochemical marker (e.g., a Nestin marker).

The data features for the cHCC-CCA machine-learning model may be filtered, for example to remove any highly correlated features or low prevalence features (i.e., rare features that are infrequently identified in CCA or HCC cancers). The correlation cutoff threshold can be set as desired by the user (for example, a cutoff threshold of about 0.8 or higher, or about 0.9 or higher). A low prevalence threshold may also be selected as desired by the user. An exemplary list of data features that may be used to characterize the cHCC-CCA as HCC-like or CCA-like is provided in Table 1. The data features that are used may include 1 or more, 2 or more, 3 or more, 5 or more, 10 or more, 20 or more, 30 or more, 50 or more, 75 or more, 100 or more, 125 or more 125 or more, 150 or more, or all of the features listed in Table 1.

TABLE 1

| | | | |
|---|---|---|---|
| ACVR1B functional | APC functional | ARID1A functional | ASXL1 functional |
| variant status | variant status | variant status | variant status |
| ATM functional | BAP1 functional | BCOR functional | BRAF functional |
| variant status | variant status | variant status | variant status |
| BRCA1 functional | BRCA2 functional | CCND3 functional | CCNE1 functional |
| variant status | variant status | variant status | variant status |
| CDK4 functional | CDK6 functional | CDKN2A functional | CDKN2B functional |
| variant status | variant status | variant status | variant status |
| CHEK2 functional | CREBBP functional | CTNNB1 functional | DNMT3A functional |
| variant status | variant status | variant status | variant status |
| EGFR functional | ERBB2 functional | ERBB3 functional | ERRFI1 functional |
| variant status | variant status | variant status | variant status |
| FBXW7 functional | FGF3 functional | FGFR2 functional | FGFR3 functional |
| variant status | variant status | variant status | variant status |
| GATA6 functional | GNAS functional | HGF functional | IDH1 functional |
| variant status | variant status | variant status | variant status |
| IDH2 functional | KDM6A functional | KEAP1 functional | KMT2D functional |
| variant status | variant status | variant status | variant status |
| KRAS functional | LYN functional | MAP2K4 functional | MCL1 functional |
| variant status | variant status | variant status | variant status |
| MDM2 functional | MDM4 functional | MET functional | MLH1 functional |
| variant status | variant status | variant status | variant status |
| MUTYH functional | MUTYH functional | MYC functional | NF1 functional |
| variant status | variant status | variant status | variant status |
| NF2 functional | NFE2L2 functional | NOTCH2 functional | NRAS functional |
| variant status | variant status | variant status | variant status |
| NTRK1 functional | PBRM1 functional | PIK3C2B functional | PIK3CA functional |
| variant status | variant status | variant status | variant status |
| PRKN functional | PTEN functional | RB1 functional | RBM10 functional |
| variant status | variant status | variant status | variant status |
| RICTOR functional | RNF43 functional | SETD2 functional | SF3B1 functional |
| variant status | variant status | variant status | variant status |
| SMAD4 functional | SMARCA4 functional | STK11 functional | TERT functional |
| variant status | variant status | variant status | variant status |
| TP53 functional | TSC1 functional | TSC2 functional | VEGFA functional |
| variant status | variant status | variant status | variant status |
| ZNF217 functional | 1p chromosomal arm | 1q chromosomal arm | 2p chromosomal arm |
| variant status | gain status | gain status | gain status |
| 2q chromosomal arm | 3p chromosomal arm | 3q chromosomal arm | 4p chromosomal arm |
| gain status | gain status | gain status | gain status |
| 4q chromosomal arm | 5p chromosomal arm | 5q chromosomal arm | 6p chromosomal arm |
| gain status | gain status | gain status | gain status |
| 6q chromosomal arm | 7p chromosomal arm | 7q chromosomal arm | 8p chromosomal arm |
| gain status | gain status | gain status | gain status |
| 8q chromosomal arm | 9p chromosomal arm | 9q chromosomal arm | 10p chromosomal arm |
| gain status | gain status | gain status | gain status |
| 10q chromosomal arm | 11p chromosomal arm | 11q chromosomal arm | 12p chromosomal arm |
| gain status | gain status | gain status | gain status |
| 12q chromosomal arm | 13q chromosomal arm | 14q chromosomal arm | 15q chromosomal arm |
| gain status | gain status | gain status | gain status |
| 16p chromosomal arm | 16q chromosomal arm | 17p chromosomal arm | 17q chromosomal arm |
| gain status | gain status | gain status | gain status |
| 18p chromosomal arm | 18q chromosomal arm | 19p chromosomal arm | 19q chromosomal arm |
| gain status | gain status | gain status | gain status |
| 20p chromosomal arm | 20q chromosomal arm | 21p chromosomal arm | 21q chromosomal arm |
| gain status | gain status | gain status | gain status |
| 22q chromosomal arm | 1p chromosomal arm | 2p chromosomal arm | 2q chromosomal arm |
| gain status | loss status | loss status | loss status |
| 3p chromosomal arm | 3q chromosomal arm | 4p chromosomal arm | 4q chromosomal arm |
| loss status | loss status | loss status | loss status |
| 5p chromosomal arm | 5q chromosomal arm | 6p chromosomal arm | 6q chromosomal arm |
| loss status | loss status | loss status | loss status |
| 7p chromosomal arm | 7q chromosomal arm | 8p chromosomal arm | 8q chromosomal arm |
| loss status | loss status | loss status | loss status |
| 9p chromosomal arm | 9q chromosomal arm | 10p chromosomal arm | 10q chromosomal arm |
| loss status | loss status | loss status | loss status |
| 11p chromosomal arm | 11q chromosomal arm | 12p chromosomal arm | 12q chromosomal arm |
| loss status | loss status | loss status | loss status |
| 13q chromosomal arm | 14q chromosomal arm | 15q chromosomal arm | 16p chromosomal arm |
| loss status | loss status | loss status | loss status |
| 16q chromosomal arm | 17p chromosomal arm | 17q chromosomal arm | 18p chromosomal arm |
| loss status | loss status | loss status | loss status |
| 18q chromosomal arm | 19p chromosomal arm | 19q chromosomal arm | 20p chromosomal arm |
| loss status | loss status | loss status | loss status |
| 20q chromosomal arm | 21p chromosomal arm | 21q chromosomal arm | Tumor Purity |
| loss status | loss status | loss status | |
| HBV status | Genomic ancestry | gLOH | MSI |
| TMB | | | |

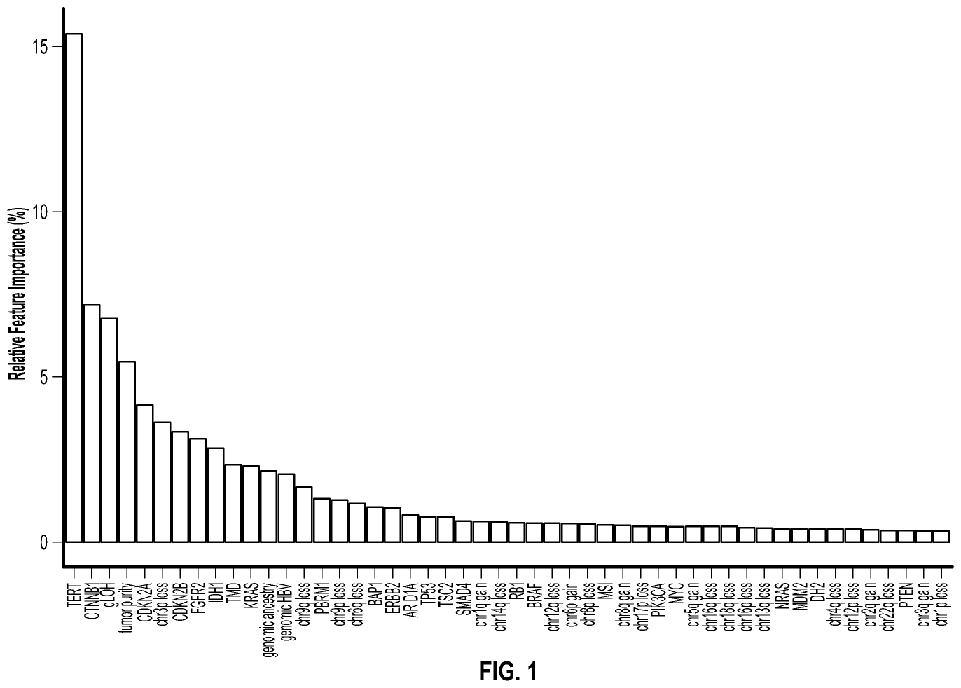

Features may be ranked based on importance, and in some embodiments, features of higher importance are used to train and/or use the cHCC-CCA machine-learning model. For example, in some embodiments, the top most important feature, the top two most important features, the top 3 most important features, the top 5 most important features, the top 10 most important features, the top 20 most important features, the top 30 most important features, the top 50 most important features, the top 75 most important features, the top 100 most important features, the top 125 most important features, or the top 150 most important features are used. In a logistic regression model, if used according to the method, the features need not be equally weighted, and different weights may be assigned to the various data features. The weights may be assigned, for example, by training the cHCC-CCA machine-learning model using hepatocellular carcinoma (HCC) data comprising from a plurality of HCC samples and cholangiocarcinoma (CCA) data from a plurality of CCA samples. The weights may be assigned, for example, based on the relative importance of each feature. By way of example, FIG. 1 shows relative feature importance of an exemplary set of data features used to train an exemplary cHCC-CCA machine-learning model (the top 50 of 157 features are shown). Feature that are unimportant are optionally omitted from the test data and/or HCC or CCA data.

In an example, the test data, the HCC data, and the CCA data each comprise genomic data comprising a TERT functional variant status (e.g., a presence or an absence of a TERT functional variant).

In another example, the test data, the HCC data, and the CCA data each comprise genomic data comprising a TERT functional variant status (e.g., a presence or an absence of a TERT functional variant) and a CTNNB1 functional variant status (e.g., a presence or an absence of a CTNNB1 functional variant).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), and a gLOH.

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, and a tumor purity.

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, and a CDKN2A functional variant status (e.g., a presence or an absence of a CDKN2A functional variant).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), and a chromosomal aneuploidy status for a 3p chromosomal arm loss (e.g., a presence or an absence of the 3p chromosomal arm loss).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), and a CDKN2B functional variant status (e.g., a presence or an absence of a CDKN2B functional variant).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), and a FGFR2 functional variant status (e.g., a presence or an absence of a FGFR2 functional variant).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), and a IDH1 functional variant status (e.g., a presence or an absence of a IDH1 functional variant).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), and a TMB.

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), a TMB, and a KRAS functional variant status (e.g., a presence or an absence of a KRAS functional variant).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), a TMB, a KRAS functional variant status (e.g., a presence or an absence of a KRAS functional variant), and an ancestry stats (such as a genomic ancestry status).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), a TMB, a KRAS functional variant status (e.g., a presence or an absence of a KRAS functional variant), an ancestry stats (such as a genomic ancestry status), and an HBV status (such as a presence or absence of genomic HBV DNA).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), a TMB, a KRAS functional variant status (e.g., a presence or an absence of a KRAS functional variant), an ancestry stats (such as a genomic ancestry status), an HBV status (such as a presence or absence of genomic HBV DNA), and a chromosomal aneuploidy status for a 9q chromosomal arm loss (e.g., a presence or an absence of the 9qchromosomal arm loss).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), a TMB, a KRAS functional variant status (e.g., a presence or an absence of a KRAS functional variant), an ancestry stats (such as a genomic ancestry status), an HBV status (such as a presence or absence of genomic HBV DNA), a chromosomal aneuploidy status for a 9q chromosomal arm loss (e.g., a presence or an absence of the 9qchromosomal arm loss), and a PBRM1 functional variant status (e.g., a presence or an absence of a PBRM1 functional variant).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), a TMB, a KRAS functional variant status (e.g., a presence or an absence of a KRAS functional variant), an ancestry stats (such as a genomic ancestry status), an HBV status (such as a presence or absence of genomic HBV DNA), a chromosomal aneuploidy status for a 9q chromosomal arm loss (e.g., a presence or an absence of the 9q chromosomal arm loss), a PBRM1 functional variant status (e.g., a presence or an absence of a PBRM1 functional variant), and a chromosomal aneuploidy status for a 9p chromosomal arm loss (e.g., a presence or an absence of the 9p chromosomal arm loss).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), a TMB, a KRAS functional variant status (e.g., a presence or an absence of a KRAS functional variant), an ancestry stats (such as a genomic ancestry status), an HBV status (such as a presence or absence of genomic HBV DNA), a chromosomal aneuploidy status for a 9q chromosomal arm loss (e.g., a presence or an absence of the 9q chromosomal arm loss), a PBRM1 functional variant status (e.g., a presence or an absence of a PBRM1 functional variant), a chromosomal aneuploidy status for a 9p chromosomal arm loss (e.g., a presence or an absence of the 9p chromosomal arm loss), and a chromosomal aneuploidy status for a 6q chromosomal arm loss (e.g., a presence or an absence of the 6q chromosomal arm loss).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), a TMB, a KRAS functional variant status (e.g., a presence or an absence of a KRAS functional variant), an ancestry stats (such as a genomic ancestry status), an HBV status (such as a presence or absence of genomic HBV DNA), a chromosomal aneuploidy status for a 9q chromosomal arm loss (e.g., a presence or an absence of the 9q chromosomal arm loss), a PBRM1 functional variant status (e.g., a presence or an absence of a PBRM1 functional variant), a chromosomal aneuploidy status for a 9p chromosomal arm loss (e.g., a presence or an absence of the 9p chromosomal arm loss), a chromosomal aneuploidy status for a 6q chromosomal arm loss (e.g., a presence or an absence of the 6q chromosomal arm loss), and a BAP1 functional variant status (e.g., a presence or an absence of a BAP1 functional variant).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), a TMB, a KRAS functional variant status (e.g., a presence or an absence of a KRAS functional variant), an ancestry stats (such as a genomic ancestry status), an HBV status (such as a presence or absence of genomic HBV DNA), a chromosomal aneuploidy status for a 9q chromosomal arm loss (e.g., a presence or an absence of the 9q chromosomal arm loss), a PBRM1 functional variant status (e.g., a presence or an absence of a PBRM1 functional variant), a chromosomal aneuploidy status for a 9p chromosomal arm loss (e.g., a presence or an absence of the 9p chromosomal arm loss), a chromosomal aneuploidy status for a 6q chromosomal arm loss (e.g., a presence or an absence of the 6q chromosomal arm loss), a BAP1 functional variant status (e.g., a presence or an absence of a BAP1 functional variant), and a ERBB2 functional variant status (e.g., a presence or an absence of a ERBB2 functional variant).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), a TMB, a KRAS functional variant status (e.g., a presence or an absence of a KRAS functional variant), an ancestry stats (such as a genomic ancestry status), an HBV status (such as a presence or absence of genomic HBV DNA), a chromosomal aneuploidy status for a 9q chromosomal arm loss (e.g., a presence or an absence of the 9q chromosomal arm loss), a PBRM1 functional variant status (e.g., a presence or an absence of a PBRM1 functional variant), a chromosomal aneuploidy status for a 9p chromosomal arm loss (e.g., a presence or an absence of the 9p chromosomal arm loss), a chromosomal aneuploidy status for a 6q chromosomal arm loss (e.g., a presence or an absence of the 6q chromosomal arm loss), a BAP1 functional variant status (e.g., a presence or an absence of a BAP1 functional variant), a ERBB2 functional variant status (e.g., a presence or an absence of a ERBB2 functional variant), and a ARID1A functional variant status (e.g., a presence or an absence of a ARID1A functional variant).

In another example, the test data, the HCC data, and the CCA data each comprise genomic data comprising a TERT functional variant status (e.g., a presence or an absence of a TERT functional variant), a CTNNB1 functional variant status (e.g., a presence or an absence of a CTNNB1 functional variant), a gLOH, a tumor purity, a CDKN2A functional variant status (e.g., a presence or an absence of a CDKN2A functional variant), a chromosomal aneuploidy status for a 3p chromosomal arm loss (e.g., a presence or an absence of the 3p chromosomal arm loss), a CDKN2B functional variant status (e.g., a presence or an absence of a CDKN2B functional variant), a FGFR2 functional variant status (e.g., a presence or an absence of a FGFR2 functional variant), a IDH1 functional variant status (e.g., a presence or an absence of a IDH1 functional variant), a TMB, a KRAS functional variant status (e.g., a presence or an absence of a KRAS functional variant), an ancestry stats (such as a genomic ancestry status), an HBV status (such as a presence or absence of genomic HBV DNA), a chromosomal aneuploidy status for a 9q chromosomal arm loss (e.g., a presence or an absence of the 9q chromosomal arm loss), a PBRM1 functional variant status (e.g., a presence or an absence of a PBRM1 functional variant), a chromosomal aneuploidy status for a 9p chromosomal arm loss (e.g., a presence or an absence of the 9p chromosomal arm loss), a chromosomal aneuploidy status for a 6q chromosomal arm loss (e.g., a presence or an absence of the 6q chromosomal arm loss), a BAP1 functional variant status (e.g., a presence or an absence of a BAP1 functional variant), a ERBB2 functional variant status (e.g., a presence or an absence of a ERBB2 functional variant), a ARID1A functional variant status (e.g., a presence or an absence of a ARID1A functional variant), and a TP53 functional variant status (e.g., a presence or an absence of a TP53 functional variant).

### Samples and Feature Determination

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

The disclosed methods and systems may be applied to the analysis of nucleic acids extracted from any of variety of tissue samples (or disease states thereof), e.g., solid tissue samples, soft tissue samples, metastatic lesions, or liquid biopsy samples.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing”, and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S.L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions—deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix—Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Cancer Characterization Methods

The cancer, such as the cHCC-CCA, is characterized using a trained cHCC-CCA machine-learning model that is configured to characterize the cancer as HCC-like or CCA-like. The cHCC-CCA machine-learning model is trained using data features from a plurality of HCC samples (i.e., HCC data) and data features from a plurality of CCA samples (i.e., CCA data). Test data associated with a sample from a subject with cancer is inputted into the trained cHCC-CCA machine-learning model, which then classifies the cHCC-CCA as HCC-like or CCA-like based on the test data. Optionally, the cHCC-CCA machine-learning model may be further configured to characterize the cancer as ambiguous. The cHCC-CCA machine-learning model may be a probabilistic classifier. The probabilistic classifier can be configured to compute a probability that the cancer or sample is HCC-like or a probability that the cancer or sample is CCA-like. Based on the probability or probabilities outputted from the cHCC-CCA machine-learning model, the cancer or sample can be called as being CCA-like or HCC-like, or ambiguous (for example, if the neither the probability that the test cancer sample is CCA-like nor the probability that the cancer or sample is HCC-like is above a predetermined probability threshold). The test data, HCC data, and CCA data can include the data features discussed herein.

The characterization method may be a computer-implemented method using a specifically designed machine or system that includes a trained cHCC-CCA machine-learning model, which may be stored on a non-transitory computer readable memory of the computer or system. The computer generally includes one or more processors that can access the memory. The one or more processors can receive test data, which may also be stored on the memory. The one or more processors can access the trained cHCC-CCA machine-learning model, and can input the test data into the model. The one or more processors and the trained cHCC-CCA machine-learning model can then characterize the cancer as HCC-like or CCA-like.

The cHCC-CCA model may be a classification model, which can classify the cHCC-CCA as HCC-like or CCA-like. The model may be an ensemble model, which optionally implements a bootstrap-aggregation method (“bagging”). The model may be a tree-based model, such as a tree-based ensemble model. By way of example, the cHCC-CCA machine-learning model may be random-forest model.

Other machine-learning paradigms may be used for the cHCC-CCA machine-learning model. For example, the cHCC-CCA machine learning model may be a regression-based model (such as a logistic regression model), a regularization-based model (such as an elastic net model or a ridge regression model), an instance-based model (such as a support vector machine or a k-nearest neighbor model), a Bayesian-based model (such as a naïve-based model or a Gaussian naïve-based model) a clustering-based model (such as an expectation maximization model), an ensemble-based model (such as an adaptive boosting (AdaBoost) model, a bagging model, or a gradient boosting machine model), or a neural-network based model (such as a back propagation network, or a stochastic gradient descent network). Deep learning models (such as convolutional neural networks, recurrent neural networks, or auto-encoders) may also be used for the cHCC-CCA machine-learning model.

The cHCC-CCA machine-learning model may classify the cancer of the subject as CCA-like or HCC-like. Optionally, the cHCC-CCA machine-learning model may classify the cHCC-CCA of the subject as CCA-like, HCC-like, or ambiguous. For example, the cHCC-CCA machine-learning model may classify the cHCC-CCA as ambiguous if it cannot classify the cHCC-CCA as HCC-like or CCA-like with sufficiently high confidence or probability. The confidence or probability threshold may be set by the user as desired, given the tolerance for inaccurate classification.

The cHCC-CCA machine learning model may be configured to assign a probability to the cancer of the subject, for example a probability that the cHCC-CCA is HCC-like, a probability that the cHCC-CCA is CCA-like, or both.

A report may be generated that identifies the cancer as HCC-like or CCA-like (or ambiguous). The report may be, for example an electronic medical record or a printed report, which can be transmitted to the subject or a healthcare provider (doctor, clinic, etc.) for the subject. The report may be used to make healthcare decisions, such as the method by which the cancer in the subject is treated.

The report may be displayed on an electronic display or customized interface. For example, in some embodiments, the computer-implemented method may automatically generate the report, and may automatically display the generated report on an electronic display or customized interface.

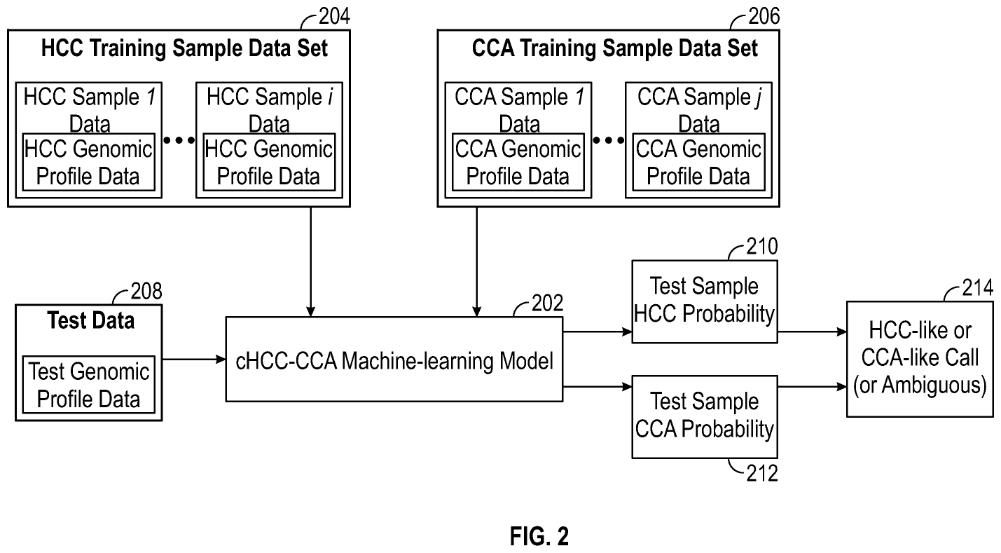

FIG. 2 shows an exemplary method for training and operating the cHCC-CCA machine-learning model 202 configured to classify a cancer as HCC-like or CCA-like. The cHCC-CCA machine-learning model 202 is trained using HCC training sample data set 204 and CCA training sample data set 206. The HCC training sample data set 204 includes HCC data for a plurality of HCC training samples (i.e., HCC sample 1 through HCC sample i). Each HCC sample is associated with HCC data features for the HCC, which can include HCC genomic data features for the HCC. The HCC data features are labeled as being associated with the HCC. Similarly, the CCA training sample data set 206 includes CCA data for a plurality of CCA training samples (i.e., CCA sample 1 through CCA sample j). Each CCA sample is associated with CCA data features for the CCA, which can include CCA genomic data features for the CCA. The CCA data features are labeled as being associated with the CCA.

Test data 208 associated with a sample (e.g., a cancer test sample) from the subject in inputted into the trained cHCC-CCA machine-learning model 202. The test data can include genomic data for the sample associated with the cHCC-CCA. The trained cHCC-CCA machine-learning model 202 may then classify the cancer as HCC-like or CCA-like. For example, the cHCC-CCA machine-learning model 202 may determine a probability that the cancer or sample is HCC-like 210 and a probability that the cancer or sample is CCA-like 212. The probabilities 210 and 212 are optionally inputted into a HCC/CCA calling module 214. The HCC/CCA calling module 214 can call the cancer as HCC-like or CCA-like based on the probabilities 210 and 212. For example, if the probability that the cancer or sample is HCC-like 210 is greater than the probability that the cancer or sample is CCA-like 212, then the cancer or sample can be called as HCC-like. If the probability that the cancer or sample is CCA-like 212 is greater than the probability that the cancer or sample is HCC-like 210, then the cancer or sample can be called as CCA-like. Optionally, if neither of probabilities 210 and 212 are above a predetermined threshold, the cancer or sample can be called as ambiguous.

The methods described herein may be implemented using one or more computer systems. Such computer systems can include one or more programs configured to execute one or more processors for the computer system to perform such methods. One or more steps of the computer-implemented methods may be performed automatically. The computer system may include one or more computing nodes. For example, a system may include two or more computing nodes (e.g., servers, computers, routers, or other types of electronic devices that include a network interface), which may be connected and configured to communicate and execute the methods over said network on one or more computing nodes of the network.

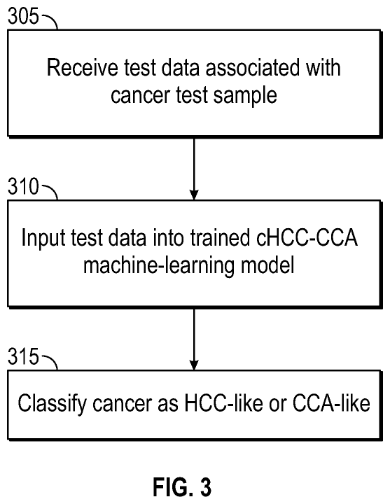

FIG. 3 is a flowchart of an exemplary computer-implemented method of characterizing a cancer, such as cHCC-CCA, which may be performed at an electronic device or system. At 305, test data (which can include genomic data for the sample) associated with a sample from a subject with the cancer is received at one or more processors. The test data may be stored on a computer-readable memory accessible by the one or more processors. In some embodiments, the test data is received from another electronic device and stored on the memory. For example, a healthcare provider may upload the genomic data for the sample to a server, and the test genomic profile may be stored in the memory. In some embodiments, sequencing data is uploaded onto a server, and the sequencing data is analyzed to generate the genomic data for the sample, for example using a genomic data generation module.

At step 310, the test data is inputted into a trained cHCC-CCA machine-learning model using the one or more processors. The cHCC-CCA machine-learning module may be trained using HCC data (which can include HCC genomic data) for a plurality of HCC training samples and CCA data (which can include CCA genomic data) for a plurality of CCA training samples, and can therefore be configured to classify the cancer, based on the test data, as CCA-like or HCC-like. In some implementations of the method, the cHCC-CCA machine-learning module is configured to classify the cancer as HCC-like, CCA-like, or ambiguous. The trained cHCC-CCA machine-learning model may be stored on the non-transitory computer-readable memory, which is accessible by the one or more processors.

At 315, the cancer is characterized as HCC-like or CCA-like using the one or more processors and the cHCC-CCA machine-learning module. Optionally, after the cancer is characterized as HCC-like or CCA-like, a report can be generated that indicates whether the cHCC-CCA is characterized as HCC-like or CCA-like (or ambiguous). The report may be automatically generated. In some embodiments, the report may be automatically displayed on an electronic display and/or automatically provided to the subject or a healthcare provider for the subject.

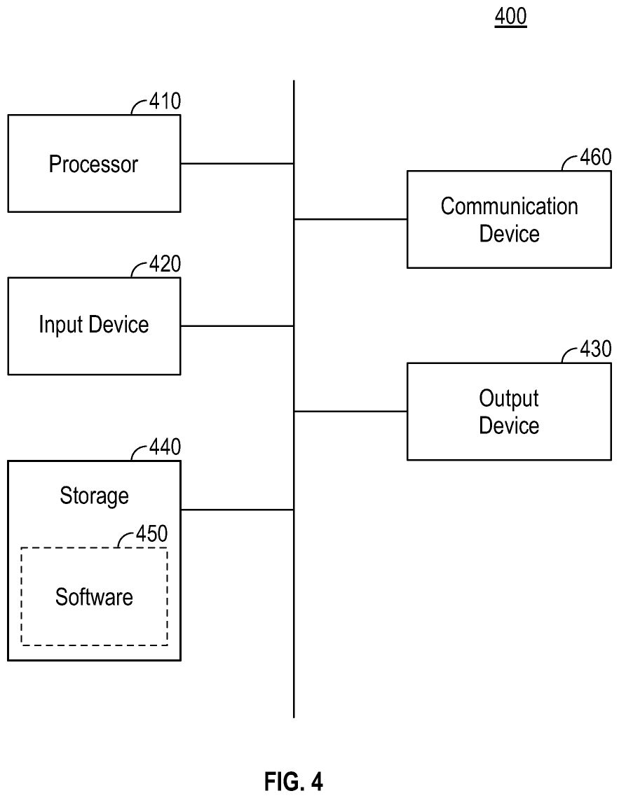

FIG. 4 shows an example of a computing device in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more of processor 410, input device 420, output device 430, storage 440, and communication device 460. Input device 420 and output device 430 can generally correspond to those described above, and can either be connectable or integrated with the computer.

Input device 420 can be any suitable device that provides input, such as a touch screen, keyboard or keypad, mouse, or voice-recognition device. Output device 430 can be any suitable device that provides output, such as a display, touch screen, haptics device, or speaker.

Storage 440 can be any suitable device that provides storage, such as an electrical, magnetic or optical memory including a RAM, cache, hard drive, or removable storage disk. Communication device 460 can include any suitable device capable of transmitting and receiving signals over a network, such as a network interface chip or device. The components of the computer can be connected in any suitable manner, such as via a physical bus or wirelessly.

Software 450, which can be stored in storage 440 and executed by processor 410, can include, for example, the programming that embodies the functionality of the present disclosure (e.g., as embodied in the devices as described above).

Software 450 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 440, that can contain or store programming for use by or in connection with an instruction execution system, apparatus, or device.

Software 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 400 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can implement any operating system suitable for operating on the network. Software 450 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

### Treatment Methods

Characterization of the cancer, such as a cHCC-CCA, in the subject is particularly useful for selecting an effective treatment. Cancers that are classified as HCC-like can be treated as though they are HCC cancers, and cHCC-CCA cancers that are characterized as CCA-like can be treated as though they are CCA cancers. CCA cancers and HCC cancers may be treated differently, and it is important for a healthcare provider or the subject to understand how the cancer, such as a cHCC-CCA, should be characterized so that it may be effectively treated. Thus, a method of treating a subject with cancer can include obtaining a characterization of the cancer as HCC-like or CCA-like, wherein the cancer is characterized according to the characterization method described herein; and administering a treatment to the subject, wherein the treatment is selected to treat HCC if the cancer is characterized as HCC-like, and the treatment is selected to treat CCA if the cancer is characterized as CCA-like.

The method of treating a subject with a cancer can include obtaining a characterization of the cancer as HCC-like or CCA-like. To obtain this characterization, the cHCC-CCHA machine-learning model described herein may be used. Test data (which can include genomic data for the sample) associated with the cancer may be inputted into the cHCC-CCA machine-learning model, which is configured to characterize the cancer as CCA-like or HCC-like based on the test data. The cHCC-CCA machine-learning model is trained using HCC data (which can include HCC genomic data) from a plurality of HCC training samples and CCA data (which can include CCA genomic data) from a plurality of CCA training samples. The characterization may be obtained, for example, by operating the cHCC-CCA machine-learning model, or by receiving the results from another that operated the cHCC-CCA machine-learning model.

The treatment method may include obtaining the test data. A test sample may be obtained from the subject (e.g., a subject having cancer), and nucleic acid molecules may be derived from the test sample. The test sample may be, for example, a solid tissue biopsy of the cancer, and nucleic acids may be isolated from the solid tissue sample. Optionally, the test sample may be preserved, for example by freezing the test sample or fixing the sample (e.g., by forming a FFPE sample) prior to isolating the nucleic acid molecules. Alternatively, the test sample is a liquid biopsy sample (e.g., a blood, plasma, cerebrospinal fluid, sputum, stool, urine, saliva, or other liquid sample from the subject), and nucleic acids, including ctDNA, may be obtained from the liquid sample. The nucleic acids from the sample may be sequenced to generate the sequencing data, which can be analyzed to generate the genomic data for the sample.

Obtaining the characterization of the cancer as HCC-like or CCA-like can include inputting the test data into the trained cHCC-CCA machine-learning model, and characterizing, using the trained cHCC-CCA machine-learning model, the cancer as HCC-like or CCA-like based on the test data. Alternatively, obtaining the characterization of the cancer as HCC-like or CCA-like may include receiving a report from another entity. The report may be generated by the other entity, and the report can include a characterization of the cancer as HCC-like or CCA-like, wherein the characterization is generated using the characterization method described herein. In some embodiments, the report includes a probability that the cancer is CCA-like and/or a probability that the cancer is HCC-like, and a final characterization can be made based on the probabilities.

Once a characterization of the cancer as HCC-like or CCA-like has been made, a treatment can be selected based on the characterization. If the cancer is characterized as HCC-like, a treatment that is effective in treating HCC is selected. If the cancer is characterized as CCA-like, a treatment that is effective in treating CCA is selected. The selected treatment can then be administered to the subject to treat the cHCC-CCA.

Effective treatments for HCC can include one or more of a localized therapy (such as local surgery or local radiotherapy), a multi-targeted tyrosine kinase inhibitor (TKI), or an immunotherapy. Local radiation therapy may include, for example, external beam radiation (EBRT), stereotactic body radiation (SBRT), charged particle therapy (such as proton beam therapy (PBT)), selective internal radiation therapy (SIRT), or ablation therapy (such as radiofrequency ablation (RFA) or microwave ablation (MWA)). Localized therapy may also include, for example, other treatments such as percutaneous ethanol injection therapy (PEIT), transarterial radioembolization (TARE), transarterial chemoembolization (TACE), highly-focused ultrasound (HIFU), irreversible electroporation (IRE), or more invasive surgical procedures (such as liver resection or liver transplantation). Exemplary multi-targeted TKIs include axitinib, brivanib, cabozantinib, cediranib, donofenib, dovitinib, lenvatinib, linifanib, nintedanib, regorafenib, sorafenib, and sunitinib. Exemplary immunotherapies include immune checkpoint inhibitors, such as inhibitors against cytotoxic T-lymphocyte antigen-4 (CTLA4), programmed death-1 (PD-1), or programmed death-1 ligand (PD-L1). Without being limited, the immunotherapy may include an antibody or fragment targeting an immune checkpoint, such as, for example, an anti-CTLA4 antibody (such as tremelimumab or ipilimumab), an anti-PD-1 antibody (such as nivolumab, pembrolizumab, camrelizumab, or tislelizumab), or an anti-PD-L1 antibody (such as avelumab, atezolizumab, or durvalumab). Other therapies for treating HCC are described in Marrero et al., Diagnosis, Staging, and Management of Hepatocellular Carcinoma: 2018 Practice Guidance by the American Association for the Study of Liver Diseases, Hepatology, vol. 68, no. 2, pp. 723-750 (2018); Huang et al., Targeted therapy for hepatocellular carcinoma, Signal Transduction and Targeted Therapy, vol. 5, article 146 (2020); and Draper, A Concise Review of the Changing Landscape of Hepatocellular Carcinoma, Hepatocellular Carcinoma: Examining the Latest Evidence for Managed Care, American Journal of Managed Care, Supplement., vol. 26, no. 10, pp. S211-S219 (2020).

Effective treatments for CCA can include a chemotherapy or a targeted therapy (e.g., a kinase-specific inhibitor). Chemotherapy may include one or more of a fluoropyrimidine (e.g., gemcitabine, capecitabine, doxifluridine, fluorouracil, irinotecan, or tegafur (optionally in combination with uracil)), a platinum agent (e.g., cisplatin or oxaliplatin), or a taxane (such as docetaxel or paclitaxel). Exemplary targeted therapies include target-specific kinase inhibitors, such as an IDH1 inhibitor (such as ivosidenib), an FGFR2 inhibitor (such as pemigatinib, infigratinib, derazantinib, or bemarituzumab), a MEK inhibitor (such as selumetinib), mTOR inhibitor (such as everolimus), a TRF inhibitor, or a WNT inhibitor. A subject treated with an IDH1 inhibitor may have an IDH1 mutation. A subject treated with an FGFR2 inhibitor may have an FGFR2 mutation. A subject treated with a MEK inhibitor or an mTOR inhibitor may have a KRAS mutation. Other therapies for treating CCA are described in Banales et al., Cholangiocarcinoma 2020: the next horizon in mechanisms and management, Nature Reviews Gastroenterology & Hepatology, vol. 17, p. 557-588 (2020).

### EXEMPLARY EMBODIMENTS

The following embodiments are exemplary and are not intended to limit the scope of the invention described herein.

Embodiment 1. A method comprising:
generating genomic data for a sample from a subject having cancer, comprising: providing a plurality of nucleic acid molecules obtained from the sample;
ligating one or more adapters to one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules; sequencing, by a sequencer, the captured nucleic acid molecules to obtain a plurality of sequence reads that represent the captured nucleic acid molecules;
analyzing, by one or more processors, the plurality of sequence reads to generate the test genomic data;
receiving, at one or more of the one or more processors, test data for the sample, wherein the test data comprises the genomic data for the sample;
inputting, using the at least one processor, the test data into a combined hepatocellular cholangiocarcinoma (cHCC-CCA) machine-learning model trained using hepatocellular carcinoma (HCC) data comprising HCC genomic data from a plurality of HCC samples and cholangiocarcinoma (CCA) data comprising CCA genomic data from a plurality of CCA samples, wherein the cHCC-CCA machine-learning model is configured to classify the sample, based on the test data, as CCA-like, or HCC-like, or ambiguous; and
classifying, by the at least one processor using the cHCC-CCA machine-learning model, the sample as HCC-like, or CCA-like, or ambiguous.

Embodiment 2. The method of embodiment 1, wherein the one or more adapters comprise amplification primers, flow cell adaptor sequences, substrate adapter sequences, or sample index sequences.

Embodiment 3. The method embodiment 1 or 2, wherein the captured nucleic acid molecules are captured from the amplified nucleic acid molecules by hybridization to one or more bait molecules.

Embodiment 4. The method of embodiment 3, wherein the one or more bait molecules comprise one or more nucleic acid molecules, each comprising a region that is complementary to a region of a captured nucleic acid molecule.

Embodiment 5. The method of any one of embodiments 1-4, wherein amplifying nucleic acid molecules comprises performing a polymerase chain reaction (PCR) amplification technique, a non-PCR amplification technique, or an isothermal amplification technique.

Embodiment 6. The method of any one of embodiments 1-5, wherein the sequencing comprises use of a massively parallel sequencing (MPS) technique, whole genome sequencing (WGS), whole exome sequencing, targeted sequencing, direct sequencing, or Sanger sequencing technique.

Embodiment 7. The method of embodiment 6, wherein the sequencing comprises massively parallel sequencing, and the massively parallel sequencing technique comprises next generation sequencing (NGS).

Embodiment 8. The method of any one of embodiments 1-7, wherein the sequencer comprises a next generation sequencer.

Embodiment 9. A method, comprising:
receiving, at one or more processors, test data comprising genomic data for a sample from a subject having cancer;
inputting, using the one or more processors, the test data into a combined hepatocellular cholangiocarcinoma (cHCC-CCA) machine-learning model trained using hepatocellular carcinoma (HCC) data comprising HCC genomic data from a plurality of HCC samples and cholangiocarcinoma (CCA) data comprising CCA genomic data from a plurality of CCA samples, wherein the cHCC-CCA machine-learning model is configured to classify the sample, based on the test data, as CCA-like, HCC-like, or ambiguous; and
classifying, using the one or more processors and the cHCC-CCA machine-learning model, the sample as HCC-like, CCA-like, or amiguous.

Embodiment 10. The method of any one of embodiments 1-9, wherein the cHCC-CCA machine-learning model is a probabilistic classifier configured to compute a probability that the sample is HCC-like or a probability that the sample is CCA-like.

Embodiment 11. The method of any one of embodiments 1-10, further comprising training the cHCC-CCA machine learning model using the HCC data and the CCA data.

Embodiment 12. The method of any one of embodiments 1-11, wherein the sample is a bile duct cancer sample.

Embodiment 13. The method of embodiment 12, wherein the bile duct cancer sample is an intrahepatic bile duct cancer sample, an extrahepatic bile duct cancer sample, a perihilar bile duct cancer sample, or a distal bile duct cancer sample.

Embodiment 14. The method of any one of embodiments 1-13, wherein the sample is a combined hepatocellular cholangiocarcinoma (cHCC-CCA) sample.

Embodiment 15. The method of any one of embodiments 1-11, wherein the cancer is a bile duct cancer sample.

Embodiment 16. The method of embodiment 15, wherein the bile duct cancer is an intrahepatic bile duct cancer, an extrahepatic bile duct cancer, a perihilar bile duct cancer, or a distal bile duct cancer.

Embodiment 17. The method of any one of embodiments 1-11, 15 and 16, wherein the cancer is a combined hepatocellular cholangiocarcinoma (cHCC-CCA) sample.

Embodiment 18. The method of any one of embodiments 1-17, wherein the genomic data for the sample, the HCC genomic data, and the CCA genomic data each comprise a tumor purity.

Embodiment 19. The method of any one of embodiments 1-18, wherein the genomic data for the sample, the HCC genomic data, and the CCA genomic data each comprises a chromosomal aneuploidy status for one or more chromosomes or chromosome arms.

Embodiment 20. The method of embodiment 19, wherein the chromosomal aneuploidy status comprises a loss status or a gain status of one or more of a 1q arm, 2q arm, 5p arm, 6p arm, 6q arm, 7q arm, 8p arm, 8q arm, 10q arm, 17p arm, 17q arm, 18q arm, 20p arm, 20q arm, 21p arm, and 22q arm.

Embodiment 21. The method of any one of embodiments 1-20, wherein the genomic data for the sample, the HCC genomic data, and the CCA genomic data each comprise a cancer cell fraction (CCF) for one or more genes, wherein the CCF for the one or more genes is differentially represented in CCA and HCC.

Embodiment 22. The method of embodiment 21, wherein the CCF for one or more genes differentially represented in CCA and HCC comprises a CCF of one or more of TP53, CTNNB1, TERT, IDH1, and BAP1.

Embodiment 23. The method of any one of embodiments 1-22, wherein the genomic data for the sample, the HCC genomic data, and the CCA genomic data each comprise a functional variant status for each of one or more genes.

Embodiment 24. The method of embodiment 23, wherein the functional variant status is a presence or an absence of the functional variant for the gene.

Embodiment 25. The method of embodiment 23 or 24, wherein the functional variant caused by a single nucleotide variant (SNV), a multiple nucleotide variant (MNV), a copy number alteration, an indel, or a rearrangement.

Embodiment 26. The method of any one of embodiments 23-25, wherein the one or more genes comprises ARID1A, BAP1, BRAF, CCND1, CDKN2A, CDKN2B, CTNNB1, ERBB2, FGFR2, IDH1, KRAS, MTAP, PBRM1, PIK3CA, PTEN, MYC, RB1, SMAD4, or TERT.

Embodiment 27. The method of any one of embodiments 23-26, wherein the one or more genes comprises ARID1A, BAP1, CDKN2A, CDKN2B, CTNNB1, FGFR2, IDH1, KRAS, PBRM1, MYC, or TERT.

Embodiment 28. The method of any one of embodiments 1-27, wherein the genomic data for the sample, the HCC genomic data, and the CCA genomic data each comprises a tumor mutational burden (TMB).

Embodiment 29. The method of embodiment 28, wherein the TMB is a continuous numeric feature.

Embodiment 30. The method of embodiment 28, wherein the TMB is a categorical feature.

Embodiment 31. The method of any one of embodiments 1-30, wherein the genomic data for the sample, the HCC genomic data, and the CCA genomic data each comprises a microsatellite instability (MSI) status.

Embodiment 32. The method of embodiment 28, wherein the MSI status is a categorical feature.

Embodiment 33. The method of any one of embodiments 1-32, wherein genomic data for the sample, the HCC genomic data, and the CCA genomic data each comprises a genome-wide loss of heterozygosity (gLOH) status.

Embodiment 34. The method of embodiment 33, wherein the gLOH status is a continuous numeric feature.

Embodiment 35. The method of embodiment 33, wherein the gLOH status is a categorical feature.

Embodiment 36. The method of any one of embodiments 1-33, wherein the test data, the HCC data, and the CCA data each comprises an ancestry status.

Embodiment 37. The method of embodiment 36, wherein the ancestry status is a genomic ancestry status.

Embodiment 38. The method of embodiment 37, wherein the genomic ancestry status is a categorical feature, wherein the categorical feature is at least one of African, Ad Mixed American, East Asian, European, or South Asian.

Embodiment 39. The method of any one of embodiments 1-38, wherein the test data, the HCC data, and the CCA data each comprise a hepatitis B virus (HBV) status.

Embodiment 40. The method of embodiment 39, wherein the HBV status is determined by detecting a presence or absence of genomic HBV DNA.

Embodiment 41. The method of any one of embodiments 1-40, wherein the test data, the HCC data, and the CCA data each further comprises one or more clinicopathological features.

Embodiment 42. The method of embodiment 41, wherein the one or more clinicopathological features comprises an age of the subject at the time the sample was obtained from the subject, a biological sex of the subject, a sample biopsy site, or a cancer metastasis status.

Embodiment 43. The method of any one of embodiments 9-42, wherein the genomic data for the sample, the HCC genomic data, and the CCA genomic data are each determined from sequencing data.

Embodiment 44. The method of embodiment 43, wherein the sequencing data is targeted sequencing data.

Embodiment 45. The method of embodiment 44, wherein the targeted sequencing data is generated using a hybrid-capture method.

Embodiment 46. The method of embodiment any one of embodiments 43-45, wherein the sequencing data is generated using massively parallel sequencing.

Embodiment 47. The method of any one of embodiments 1-46, wherein the cHCC-CCA machine-learning model is a tree-based classification model.

Embodiment 48. The method of any one of embodiments 1-47, wherein the cHCC-CCA machine-learning model is an ensemble model.

Embodiment 49. The method of any one of embodiments 1-48, wherein the cHCC-CCA machine-learning model is a bootstrap aggregated model.

Embodiment 50. The method of any one of embodiments 1-49, wherein the cHCC-CCA machine-learning model is a random-forest model.

Embodiment 51. The method of any one of embodiments 1-46, wherein the cHCC-CCA machine-learning model is a linear classification model.

Embodiment 52. The method of any one of embodiments 1-51, wherein the sample is a solid tissue biopsy sample.

Embodiment 53. The method of embodiment 52, wherein the solid tissue biopsy sample is a formalin-fixed paraffin-embedded (FFPE) sample.

Embodiment 54. The method of any one of embodiments 1-51, wherein the sample is a liquid biopsy sample comprising circulating tumor DNA (ctDNA).

Embodiment 55. The method of any one of embodiments 1-51, wherein the sample is a liquid biopsy sample comprising circulating tumor cells (CTCs).

Embodiment 56. The method of embodiment 54 or 55, wherein the sample is a liquid biopsy sample and comprises blood, plasma, cerebrospinal fluid, sputum, stool, urine, or saliva.

Embodiment 57. The method of any one of embodiments 1-56, comprising generating a report identifying the sample as HCC-like, CCA-like, or ambiguous.

Embodiment 58. The method of any one of embodiments 1-57, comprising generating a report identifying the cancer as HCC-like, CCA-like, or ambiguous.

Embodiment 59. The method of embodiment 57 or 58, comprising displaying the report on an electronic display.

Embodiment 60. The method of any one of embodiments 57-59, comprising transmitting the report to the subject or a healthcare provider for the subject.

Embodiment 61. The method of embodiment 60, wherein the report is transmitted via a computer network or a peer-to-peer connection.

Embodiment 62. The method of embodiment 60 or 61, wherein the report is an electronic medical record.

Embodiment 63. The method of any one of embodiments 1-62, further comprising obtaining the sample from the subject.

Embodiment 64. A method of selecting a treatment for a cancer in a subject, comprising:
obtaining a classification of a sample associated with the cancer as HCC-like or CCA-like, wherein the sample is classified using the method of any one of embodiments 1-63; and
selecting the treatment for the cancer, wherein the treatment is selected to effectively treat HCC if the sample is classified as HCC-like, and the treatment is selected to effectively treat CCA if the sample is classified as CCA-like.

Embodiment 65. The method of embodiment 64, further comprising administering the selected treatment to the subject.

Embodiment 66. A method of treating a cancer in a subject, comprising:
obtaining a classification of a sample from the subject as HCC-like or CCA-like, wherein the sample is classified using the method of any one of embodiments 1-60; and
administering a treatment to the subject, wherein the treatment is selected to effectively treat HCC if the sample is classified as HCC-like, and the treatment is selected to effectively treat CCA if the sample is classified as CCA-like.

Embodiment 67. The method of any one of embodiments 64-66, wherein the sample is classified as HCC-like, and the treatment comprises a localized therapy, a multi-targeted tyrosine kinase inhibitor, or an immunotherapy.

Embodiment 68. The method of embodiment 67, wherein the treatment comprises a multi-targeted tyrosine kinase inhibitor.

Embodiment 69. The method of embodiment 68, wherein the multi-targeted tyrosine kinase inhibitor comprises axitinib, brivanib, cabozantinib, cediranib, donofenib, dovitinib, lenvatinib, linifanib, nintedanib, regorafenib, sorafenib, or sunitinib.

Embodiment 70. The method of embodiment 67, wherein the treatment comprises an immunotherapy.

Embodiment 71. The method of embodiment 70, wherein the immunotherapy comprises an immune checkpoint inhibitor.

Embodiment 72. The method of embodiment 71, wherein the immune checkpoint inhibitor is tremelimumab, ipilimumab, nivolumab, pembrolizumab, camrelizumab, tislelizumab, avelumab, atezolizumab, or durvalumab.

Embodiment 73. The method of any one of embodiments 64-66, wherein the cancer is classified as CCA-like, and the treatment comprises a chemotherapy or a targeted therapy.

Embodiment 74. The method of embodiment 73, wherein the treatment comprises a chemotherapy.

Embodiment 75. The method of embodiment 74, wherein the chemotherapy comprises a fluoropyrimidine, a platinum agent, or a taxane.

Embodiment 76. The method of embodiment 75, wherein the chemotherapy comprises gemcitabine, capecitabine, doxifluridine, fluorouracil, irinotecan, tegafur, cisplatin, oxaliplatin, docetaxel, or paclitaxel.

Embodiment 77. The method of embodiment 73, wherein the treatment comprises a targeted therapy.

Embodiment 78. The method of embodiment 77, wherein the targeted therapy comprises a kinase-specific inhibitor.

Embodiment 79. The method of embodiment 77, wherein the treatment comprises an IDH1 inhibitor, an FGFR2 inhibitor, a MEK inhibitor, or an mTOR inhibitor.

Embodiment 80. The method of embodiment 79, wherein the treatment comprises an IDH1 inhibitor, wherein the cancer has an IDH1 mutation.

Embodiment 81. The method of embodiment 79 or 80, wherein the treatment comprises an IDH1 inhibitor, and wherein the IDH1 inhibitor is ivosidenib.

Embodiment 82. The method of embodiment 79, wherein the treatment comprises an FGFR2 inhibitor, wherein the cancer has a FGFR2 mutation.

Embodiment 83. The method of embodiment 79 or 82, wherein the treatment comprises an FGFR2 inhibitor, and the FGFR2 inhibitor is pemigatinib, infigratinib, derazantinib, or bemarituzumab.

Embodiment 84. The method of embodiment 79, wherein the treatment comprises an MEK inhibitor or an mTOR inhibitor, wherein the cancer has a KRAS mutation.

Embodiment 85. The method of embodiment 79 or 84, wherein the treatment comprises an MEK inhibitor, and wherein the MEK inhibitor is selumetinib.

Embodiment 86. The method of embodiment 79 or 84, wherein the treatment comprises an mTOR inhibitor, and wherein the mTOR inhibitor is everolimus.

Embodiment 87. The method of any one of embodiments 9-86, comprising sequencing nucleic acid molecules from the sample to obtain at least a portion of the genomic data for the sample.

Embodiment 88. A system, comprising:
one or more processors;
a memory; and
one or more programs, wherein the one or more programs are stored in the memory and configured to be executed by the one or more processors, the one or more programs including instructions for implementing a method, comprising:
receiving, at the one or more processors, test data comprising genomic data for a sample from a subject having cancer;
inputting, using the one or more processors, the test data into a combined hepatocellular cholangiocarcinoma (cHCC-CCA) machine-learning model trained using hepatocellular carcinoma (HCC) data comprising HCC genomic data from a plurality of HCC samples and cholangiocarcinoma (CCA) data comprising CCA genomic data from a plurality of CCA samples, wherein the cHCC-CCA machine-learning model is configured to classify the sample, based on the test data, as CCA-like, HCC-like, or ambiguous; and
classifying, using the one or more processors and the cHCC-CCA machine-learning model, the sample as HCC-like, CCA-like, or ambiguous.

Embodiment 89. The system of embodiment 88, comprising a sequencer configured to sequence nucleic acids derived from cancer test sample.

Embodiment 90. The system of embodiment 88 or 89, wherein the cHCC-CCA machine-learning model is a probabilistic classifier configured to compute a probability that the sample is HCC-like or a probability that the cancer test sample is CCA-like.

Embodiment 91. The system of any one of embodiments 88-90, wherein the one or more programs further include instructions for training the cHCC-CCA machine learning model using the HCC data and the CCA data.

Embodiment 92. The system of any one of embodiments 88-81, wherein the sample is a bile duct cancer sample.

Embodiment 93. The system of embodiment 92, wherein the bile duct cancer sample is an intrahepatic bile duct cancer sample, an extrahepatic bile duct cancer sample, a perihilar bile duct cancer sample, or a distal bile duct cancer sample.

Embodiment 94. The system of any one of embodiments 88-93, wherein the cancer sample is a combined hepatocellular cholangiocarcinoma (cHCC-CCA) sample.

Embodiment 95. The system of any one of embodiments 88-91, wherein the cancer is a bile duct cancer.

Embodiment 96. The system of embodiment 95, wherein the bile duct cancer is an intrahepatic bile duct cancer, an extrahepatic bile duct cancer, a perihilar bile duct cancer, or a distal bile duct cancer.

Embodiment 97. The system of any one of embodiments 88-91, 95, and 96, wherein the cancer is a combined hepatocellular cholangiocarcinoma (cHCC-CCA).

Embodiment 98. The system of any one of embodiments 88-97, wherein the genomic data for the sample, the HCC genomic data, and the CCA genomic data each comprise a tumor purity.

Embodiment 99. The system of any one of embodiments 88-98, wherein the genomic data for the sample, the HCC genomic data, and the CCA genomic data each comprises a chromosomal aneuploidy status for one or more chromosomes or chromosome arms.

Embodiment 100. The system of embodiment 99, wherein the chromosomal aneuploidy status comprises a loss status or a gain status of one or more of a 1q arm, 2q arm, 5p arm, 6p arm, 6q arm, 7q arm, 8p arm, 8q arm, 10q arm, 17p arm, 17q arm, 18q arm, 20p arm, 20q arm, 21p arm, and 22q arm.

Embodiment 101. The system of any one of embodiments 88-100, wherein the genomic data for the sample, the HCC genomic data, and the CCA genomic data each comprise a cancer cell fraction (CCF) for one or more genes, wherein the CCF for the one or more genes is differentially represented in CCA and HCC.

Embodiment 102. The system of embodiment 101, wherein the CCF for one or more genes differentially represented in CCA and HCC comprises a CCF of one or more of TP53, CTNNB1, TERT, IDH1, and BAP1.

Embodiment 103. The system of any one of embodiments 88-102, wherein the genomic data for the sample, the HCC genomic data, and the CCA genomic data each comprise a functional variant status for each of one or more genes.

Embodiment 104. The system of embodiment 103, wherein the functional variant status is a presence or an absence of the functional variant for the gene.

Embodiment 105. The system of embodiment 103 or 104, wherein the functional variant caused by a single nucleotide variant (SNV), a multiple nucleotide variant (MNV), a copy number alteration, an indel, or a rearrangement.

Embodiment 106. The system of any one of embodiments 103-105, wherein the one or more genes comprises ARID1A, BAP1, BRAF, CCND1, CDKN2A, CDKN2B, CTNNB1, ERBB2, FGFR2, IDH1, KRAS, MTAP, PBRM1, PIK3CA, PTEN, MYC, RB1, SMAD4, or TERT.

Embodiment 107. The system of any one of embodiments 103-106, wherein the one or more genes comprises ARID1A, BAP1, CDKN2A, CDKN2B, CTNNB1, FGFR2, IDH1, KRAS, PBRM1, MYC, or TERT.

Embodiment 108. The system of any one of embodiments 88-107, wherein the test genomic data, the HCC genomic data, and the CCA genomic data each comprises a tumor mutational burden (TMB).

Embodiment 109. The system of embodiment 108, wherein the TMB is a continuous numeric feature.

Embodiment 110. The system of embodiment 108, wherein the TMB is a categorical feature.

Embodiment 111. The system of any one of embodiments 88-110, wherein the genomic data for the sample, the HCC genomic data, and the CCA genomic data each comprises a microsatellite instability (MSI) status.

Embodiment 112. The system of embodiment 111, wherein the MSI status is a categorical feature.

Embodiment 113. The system of any one of embodiments 88-112, wherein genomic data for the sample, the HCC genomic data, and the CCA genomic data each comprises a genome-wide loss of heterozygosity (gLOH) status.

Embodiment 114. The system of embodiment 113, wherein the gLOH status is a continuous numeric feature.

Embodiment 115. The system of embodiment 113, wherein the gLOH status is a categorical feature.

Embodiment 116. The system of any one of embodiments 88-115, herein the test data, the HCC data, and the CCA data each comprises an ancestry status.

Embodiment 117. The system of embodiment 116, wherein the ancestry status is a genomic ancestry status.

Embodiment 118. The system of embodiment 117, wherein the genomic ancestry status is a categorical feature, wherein the categorical feature is at least one of African, Ad Mixed American, East Asian, European, or South Asian.

Embodiment 119. The system of any one of embodiments 88-118, wherein the test data, the HCC data, and the CCA data each comprise a hepatitis B virus (HBV) status.

Embodiment 120. The system of embodiment 119, wherein the HBV status is determined by detecting a presence or absence of genomic HBV DNA.

Embodiment 121. The system of any one of embodiments 88-120, wherein the test data, the HCC data, and the CCA data each further comprises one or more clinicopathological features.

Embodiment 122. The system of embodiment 121, wherein the one or more clinicopathological features comprises an age of the subject at the time the cancer test sample was obtained from the subject, a biological sex of the subject, a cancer test sample biopsy site, or a cancer metastasis status.

Embodiment 123. The system of any one of embodiments 88-112, wherein the genomic data for the sample, the HCC genomic data, and the CCA genomic data are each determined from sequencing data.

Embodiment 124. The system of embodiment 123, wherein the sequencing data is targeted sequencing data.

Embodiment 125. The system of embodiment 124, wherein the targeted sequencing data is generated using a hybrid-capture method.

Embodiment 126. The system of embodiment any one of embodiments 123-125, wherein the sequencing data is generated using massively parallel sequencing.

Embodiment 127. The system of any one of embodiments 88-126, wherein the cHCC-CCA machine-learning model is a tree-based classification model.

Embodiment 128. The system of any one of embodiments 88-127, wherein the cHCC-CCA machine-learning model is an ensemble model.

Embodiment 129. The system of any one of embodiments 88-128, wherein the cHCC-CCA machine-learning model is a bootstrap aggregated model.

Embodiment 130. The system of any one of embodiments 88-129, wherein the cHCC-CCA machine-learning model is a random-forest model.

Embodiment 131. The system of any one of embodiments 88-126, wherein the cHCC-CCA machine-learning model is a linear classification model.

Embodiment 132. The system of any one of embodiments 88-131, wherein the cancer test sample is a solid tissue biopsy sample.

Embodiment 133. The system of embodiment 132, wherein the solid tissue biopsy sample is a formalin-fixed paraffin-embedded (FFPE) sample.

Embodiment 134. The system of any one of embodiments 88-131, wherein the cancer test sample is a liquid biopsy sample comprising circulating tumor DNA (ctDNA).

Embodiment 135. The system of any one of embodiments 88-131, wherein the sample is a liquid biopsy sample comprising circulating tumor cells (CTCs).

Embodiment 136. The system of embodiment 134 or 135, wherein the sample is a liquid biopsy sample and comprises blood, plasma, cerebrospinal fluid, sputum, stool, urine, or saliva.

Embodiment 137. The system of any one of embodiments 88-136, wherein the one or more programs further include instructions for generating a report identifying the cancer test sample as HCC-like, CCA-like, or ambiguous.

Embodiment 138. The system of embodiment 137, wherein the one or more programs further include instructions for displaying the report on an electronic display.

Embodiment 139. The system of embodiment 137 or 138, wherein the one or more programs further include instructions for transmitting the report to the subject or a healthcare provider for the subject.

Embodiment 140. The system of embodiment 139, wherein the report is transmitted via a computer network or a peer-to-peer connection.

Embodiment 141. The system of embodiment 139 or 140, wherein the report is an electronic medical record.

Embodiment 142. A non-transitory computer-readable storage medium storing one or more programs, the one or more programs comprising instructions, which when executed by one or more processors of an electronic device, cause the electronic device to implement a method, comprising:
receiving, at the one or more processors, test data comprising genomic data for a sample from a subject with cancer;
inputting, using the one or more processors, the test data into a combined hepatocellular cholangiocarcinoma (cHCC-CCA) machine-learning model trained using hepatocellular carcinoma (HCC) data comprising HCC genomic data from a plurality of HCC samples and cholangiocarcinoma (CCA) data comprising CCA genomic data from a plurality of CCA samples, wherein the cHCC-CCA machine-learning model is configured to classify the sample, based on the test data, as CCA-like, HCC-like, or ambiguous; and
classifying, using the one or more processors and the cHCC-CCA machine-learning model, the sample as HCC-like, CCA-like, or ambiguous.

Embodiment 143. The non-transitory computer-readable storage medium of embodiment 142, wherein the cHCC-CCA machine-learning model is a probabilistic classifier configured to compute a probability that the cancer test sample is HCC-like or a probability that the cancer test sample is CCA-like.

Embodiment 144. The non-transitory computer-readable storage medium of embodiment 142 or 143, wherein the one or more programs further include instructions, which when executed by one or more processors of an electronic device, cause the electronic device to train the cHCC-CCA machine learning model using the HCC data and the CCA data.

Embodiment 145. The non-transitory computer-readable storage medium of any one of embodiments 142-144, wherein the sample is a bile duct cancer sample.

Embodiment 146. The non-transitory computer-readable storage medium of embodiment 145, wherein the bile duct cancer sample is an intrahepatic bile duct cancer sample, an extrahepatic bile duct cancer sample, a perihilar bile duct cancer sample, or a distal bile duct cancer sample.

Embodiment 147. The non-transitory computer-readable storage medium of any one of embodiments 142-146, wherein the sample is a combined hepatocellular cholangiocarcinoma (cHCC-CCA) sample.

Embodiment 148. The non-transitory computer-readable storage medium of any one of embodiments 142-144, wherein the cancer is a bile duct cancer.

Embodiment 149. The non-transitory computer-readable storage medium of embodiment 148, wherein the bile duct cancer is an intrahepatic bile duct cancer, an extrahepatic bile duct cancer, a perihilar bile duct cancer, or a distal bile duct cancer.

Embodiment 150. The non-transitory computer-readable storage medium of any one of embodiments 142-144, 148, and 149, wherein the cancer is a combined hepatocellular cholangiocarcinoma (cHCC-CCA).

Embodiment 151. The non-transitory computer-readable storage medium of any one of embodiments 142-150, wherein the genomic data for the test sample, the HCC genomic data, and the CCA genomic data each comprise a tumor purity.

Embodiment 152. The non-transitory computer-readable storage medium of any one of embodiments 142-151, wherein the genomic data for the test sample, the HCC genomic data, and the CCA genomic data each comprises a chromosomal aneuploidy status for one or more chromosomes or chromosome arms.

Embodiment 153. The non-transitory computer-readable storage medium of embodiment 152, wherein the chromosomal aneuploidy status comprises a loss status or a gain status of one or more of a 1q arm, 2q arm, 5p arm, 6p arm, 6q arm, 7q arm, 8p arm, 8q arm, 10q arm, 17p arm, 17q arm, 18q arm, 20p arm, 20q arm, 21p arm, and 22q arm.

Embodiment 154. The non-transitory computer-readable storage medium of any one of embodiments 142-153, wherein the genomic data for the test sample, the HCC genomic data, and the CCA genomic data each comprise a cancer cell fraction (CCF) for one or more genes, wherein the CCF for the one or more genes is differentially represented in CCA and HCC.

Embodiment 155. The non-transitory computer-readable storage medium of embodiment 154, wherein the CCF for one or more genes differentially represented in CCA and HCC comprises a CCF of one or more of TP53, CTNNB1, TERT, IDH1, and BAP1.

Embodiment 156. The non-transitory computer-readable storage medium of any one of embodiments 142-155, wherein the test genomic data, the HCC genomic data, and the CCA genomic data each comprise a functional variant status for each of one or more genes.

Embodiment 157. The non-transitory computer-readable storage medium of embodiment 156, wherein the functional variant status is a presence or an absence of the functional variant for the gene.

Embodiment 158. The non-transitory computer-readable storage medium of embodiment 156 or 157, wherein the functional variant caused by a single nucleotide variant (SNV), a multiple nucleotide variant (MNV), a copy number alteration, an indel, or a rearrangement.

Embodiment 159. The non-transitory computer-readable storage medium of any one of embodiments 156-158, wherein the one or more genes comprises ARID1A, BAP1, BRAF, CCND1, CDKN2A, CDKN2B, CTNNB1, ERBB2, FGFR2, IDH1, KRAS, MTAP, PBRM1, PIK3CA, PTEN, MYC, RB1, SMAD4, or TERT.

Embodiment 160. The non-transitory computer-readable storage medium of any one of embodiments 156-159, wherein the one or more genes comprises ARID1A, BAP1, CDKN2A, CDKN2B, CTNNB1, FGFR2, IDH1, KRAS, PBRM1, MYC, or TERT.

Embodiment 161. The non-transitory computer-readable storage medium of any one of embodiments 142-160, wherein the genomic data for the test sample, the HCC genomic data, and the CCA genomic data each comprises a tumor mutational burden (TMB).

Embodiment 162. The non-transitory computer-readable storage medium of embodiment 161, wherein the TMB is a continuous numeric feature.

Embodiment 163. The non-transitory computer-readable storage medium of embodiment 161, wherein the TMB is a categorical feature.

Embodiment 164. The non-transitory computer-readable storage medium of any one of embodiments 142-163, wherein the genomic data for the sample, the HCC genomic data, and the CCA genomic data each comprises a microsatellite instability (MSI) status.

Embodiment 165. The non-transitory computer-readable storage medium of embodiment 164, wherein the MSI status is a categorical feature.

Embodiment 166. The non-transitory computer-readable storage medium of any one of embodiments 142-165, wherein genomic data for the sample, the HCC genomic data, and the CCA genomic data each comprises a genome-wide loss of heterozygosity (gLOH) status.

Embodiment 167. The non-transitory computer-readable storage medium of embodiment 166, wherein the gLOH status is a continuous numeric feature.

Embodiment 168. The non-transitory computer-readable storage medium of embodiment 166, wherein the gLOH status is a categorical feature.

Embodiment 169. The non-transitory computer-readable storage medium of any one of embodiments 142-168, herein the test data, the HCC data, and the CCA data each comprises an ancestry status.

Embodiment 170. The non-transitory computer-readable storage medium of embodiment 169, wherein the ancestry status is a genomic ancestry status.

Embodiment 171. The non-transitory computer-readable storage medium of embodiment 170, wherein the genomic ancestry status is a categorical feature, wherein the categorical feature is at least one of African, Ad Mixed American, East Asian, European, or South Asian.

Embodiment 172. The non-transitory computer-readable storage medium of any one of embodiments 142-171, wherein the test data, the HCC data, and the CCA data each comprise a hepatitis B virus (HBV) status.

Embodiment 173. The non-transitory computer-readable storage medium of embodiment 172, wherein the HBV status is determined by detecting a presence or absence of genomic HBV DNA.

Embodiment 174. The non-transitory computer-readable storage medium of any one of embodiments 142-173, wherein the test data, the HCC data, and the CCA data each further comprises one or more clinicopathological features.

Embodiment 175. The non-transitory computer-readable storage medium of embodiment 174, wherein the one or more clinicopathological features comprises an age of the subject at the time the sample was obtained from the subject, a biological sex of the subject, a sample biopsy site, or a cancer metastasis status.

Embodiment 176. The non-transitory computer-readable storage medium of any one of embodiments 142-175, wherein the genomic data for the sample, the HCC genomic data, and the CCA genomic data are each determined from sequencing data.

Embodiment 177. The non-transitory computer-readable storage medium of embodiment 176, wherein the sequencing data is targeted sequencing data.

Embodiment 178. The non-transitory computer-readable storage medium of embodiment 177, wherein the targeted sequencing data is generated using a hybrid-capture method.

Embodiment 179. The non-transitory computer-readable storage medium of embodiment any one of embodiments 176-178, wherein the sequencing data is generated using massively parallel sequencing.

Embodiment 180. The non-transitory computer-readable storage medium of any one of embodiments 142-179, wherein the cHCC-CCA machine-learning model is a tree-based classification model.

Embodiment 181. The non-transitory computer-readable storage medium of any one of embodiments 142-180, wherein the cHCC-CCA machine-learning model is an ensemble model.

Embodiment 182. The non-transitory computer-readable storage medium of any one of embodiments 142-181, wherein the cHCC-CCA machine-learning model is a bootstrap aggregated model.

Embodiment 183. The non-transitory computer-readable storage medium of any one of embodiments 142-182, wherein the cHCC-CCA machine-learning model is a random-forest model.

Embodiment 184. The non-transitory computer-readable storage medium of any one of embodiments 142-179, wherein the cHCC-CCA machine-learning model is a linear classification model.

Embodiment 185. The non-transitory computer-readable storage medium of any one of embodiments 142-184, wherein the sample is a solid tissue biopsy sample.

Embodiment 186. The non-transitory computer-readable storage medium of embodiment 185, wherein the solid tissue biopsy sample is a formalin-fixed paraffin-embedded (FFPE) sample.

Embodiment 187. The non-transitory computer-readable storage medium of any one of embodiments 142-184, wherein the sample is a liquid biopsy sample comprising circulating tumor DNA (ctDNA).

Embodiment 188. The non-transitory computer-readable storage medium of any one of embodiments 142-184, wherein the sample is a liquid biopsy sample comprising circulating tumor cells (CTCs).

Embodiment 189. The non-transitory computer-readable storage medium of embodiment 187 or 188, wherein the sample is a liquid biopsy sample and comprises blood, plasma, cerebrospinal fluid, sputum, stool, urine, or saliva.

Embodiment 190. The non-transitory computer-readable storage medium of any one of embodiments 148-189, wherein the one or more programs further include instructions, which when executed by one or more processors of an electronic device, cause the electronic device to generate a report identifying the cancer test sample as HCC-like. CCA-like, or ambiguous.

Embodiment 191. The non-transitory computer-readable storage medium of embodiment 190, wherein the one or more programs further include instructions, which when executed by one or more processors of an electronic device, cause the electronic device to display the report on an electronic display.

Embodiment 192. The non-transitory computer-readable storage medium of embodiment 190 or 191, wherein the one or more programs further include instructions, which when executed by one or more processors of an electronic device, cause the electronic device to transmit the report to the subject or a healthcare provider for the subject.

Embodiment 193. The non-transitory computer-readable storage medium of embodiment 192, wherein the report is transmitted via a computer network or a peer-to-peer connection.

Embodiment 194. The non-transitory computer-readable storage medium of embodiment 192 or 193, wherein the report is an electronic medical record.

Embodiment 195. A method comprising:
generating genomic data for a sample from a subject having cancer, comprising:
providing a plurality of nucleic acid molecules obtained from the sample from a subject;
ligating one or more adapters to one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
analyzing, by one or more processors, the plurality of sequence reads to generate the genomic data for the sample;
receiving, at at least one of the one or more processors, test data for the sample, wherein the test data comprises the genomic data;
inputting, using the at least one processor, the test data into a machine-learning model trained using a first carcinoma data comprising a first carcinoma genomic data from a plurality of first carcinoma samples and a second carcinoma data comprising second carcinoma genomic data from a plurality of second carcinoma samples, wherein the first carcinoma samples are different from the second carcinoma samples, and wherein the machine-learning model is configured to classify the sample, based on the test data, as first-carcinoma-like, second-carcinoma-like, or ambiguous; and
classifying, by the at least one processor using the machine-learning model, the sample as first-carcinoma-like, second-carcinoma-like, or ambiguous.

Embodiment 196. A method, comprising:
receiving, at one or more processors, test data for a sample from a subject with cancer, wherein the test data comprises genomic data for the sample;
inputting, using the at least one processor, the test data into a machine-learning model trained using a first carcinoma data comprising a first carcinoma genomic data from a plurality of first carcinoma samples and a second carcinoma data comprising second carcinoma genomic data from a plurality of second carcinoma samples, wherein the first carcinoma samples are different from the second carcinoma samples, and wherein the machine-learning model is configured to classify the sample, based on the test data, as first-carcinoma-like, second-carcinoma-like, or ambiguous; and
classifying, by the at least one processor using the machine-learning model, the sample as first-carcinoma-like, second-carcinoma-like, or ambiguous.

### EXAMPLES

The application may be better understood by reference to the following non-limiting examples, which are provided as exemplary embodiments of the application. The following examples are presented in order to more fully illustrate embodiments and should in no way be construed as limiting the scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1

This study was conducted in accordance with the Western Institutional Review Board (IRB) approved protocol No. 20152817. Clinical cHCC-CCA (N=73), CCA (N=4975) and HCC (N=1470) cases, as diagnosed by the treating physician (and confirmed on hematoxylin and eosin-stained slides), underwent complete genomic profiling (CGP) in a CLIA-certified, NY State-approved, CAP accredited laboratory (Foundation Medicine, Inc, Cambridge MA). See Frampton et al., Development and validation of a clinical cancer genomic profiling test based on massively parallel DNA sequencing, Nature Biotechnology, vol. 31, pp. 1023-1031 (2013). Manual microdissection was performed if warranted on pathologist visual inspection. Results are N (%) unless otherwise stated. The chi-square test was used to estimate the P value for each of the 2×3 contingency tables and the Kruskal-Wallis test was used to determine the P value for the difference in tumor purity across the three diseases, using R software (R Foundation for Statistical Computing v3.6.0). The Wilcoxon-rank sum test and the Kruskal-Wallis test were used to test for differences between continuous variables. All P values are two-sided and multiple hypothesis testing correction was performed using the Benjamini-Hochberg procedure to calculate the false discovery rate (FDR).

Characteristics of the cases is provided in Table 2. HCC patients were predominantly male, as described previously, were enriched for younger patients and the African and East Asian genomic ancestry, while CCA patients had a comparable sex prevalence, were enriched for older patients and European genomic ancestry (Table 1). The cHCC-CCA cohort (N=73) was 71.22 male and 60.3% of European ancestry, with a median age of 62 years (range: 22 years—89+ years).

TABLE 2

| | | CCA | cHCC-CCA | HCC | |
|---|---|---|---|---|---|
| Characteristic | | (N = 4975) | (N = 73) | (N = 1470) | P value |
| Age (<=40) | | 278/4609 | 5/67 | 104/1381 | 0.13 |
| | | (6.0%) | (7.5%) | (7.5%) | |
| Biological Sex | | 2461, 2514 | 52, 21 | 1084, 386 | <0.001 |
| (Male, Female) | | (49.5%, 50.5%) | (71.2%, 28.8%) | (73.7%, 26.3%) | |
| Genomic HBV | | 93 | 8 | 154 | <0.001 |
| | | (1.9%) | (10.9%) | (10.5%) | |
| Tumor Site (Local) | | 3764/4386 | 66.72 | 1019/1340 | <0.001 |
| | | (85.8%) | (91.7%) | (76.0%) | |
| Tumor Purity (%, IQR) | | 32% | 37% | 45% | <0.001 |
| | | (20%-48%) | (20%-47%) | (28%-67%) | |
| Genomic | African | 315 | 9 | 181 | <0.001 |
| Ancestry | | (6.3%) | (12.3%) | (12.3%) | |
| | Ad Mixed | 582 | 8 | 207 | 0.05 |
| | American | (11.7%) | (10.9%) | (14.1%) | |
| | East | 329 | 10 | 154 | <0.001 |
| | Asian | (6.6%) | (13.7%) | (10.5%) | |
| | European | 3667 | 44 | 896 | <0.001 |
| | | (73.7%) | (60.3%) | (60.9%) | |
| | South | 82 | 2 | 32 | 0.33 |
| | Asian | (1.6%) | (2.7%) | (2.2%) | |

Feature Data.

CGP on 0.8-1.1 Mb of the coding genome was performed on hybridization-captured, adapter-ligation based sequencing libraries obtained from formalin-fixed paraffin-embedded (FPPE) samples to identify genomic alterations (base substitutions, small insertions/deletions, copy number alterations and rearrangements) in exons and select introns in at least 263 genes, tumor mutational burden (TMB), microsatellite instability status (MSI), genomic loss of heterozygosity (gLOH), chromosomal aneuploidy, genomic ancestry, and hepatitis B virus (HBV) status. For the purposes of training a machine-learning model, missing data across the HCC and CCA tumor types was imputed using the classification and regression tree (CART) approach, employing the MICE (v3.6.0) package from R (v3.6.0).

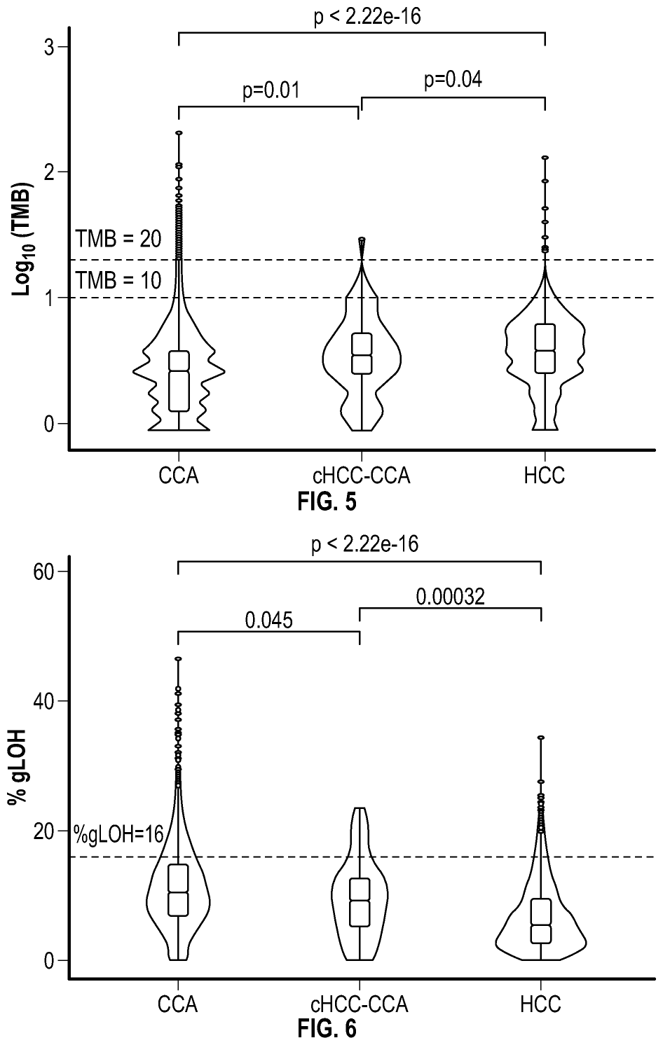

TMB was calculated as the number of non-driver somatic coding mutations per megabase of genome sequenced. See Chalmers et al., Analysis of 100,000 human cancer genomes reveals the landscape of tumor mutational burden, Genome Medicine, vol. 9, no. 34 (2017). TM/1B high (TMB-H) was defined as 20 mutations/Mb (mut/Mb) or higher. TMB was assembled for 4903/4975 CCA samples, 71/73 cHCC-CCA samples, and 1454/1470 HCC samples. TMB was encoded for the machine-learning models as a continuous numeric feature. FIG. 5 shows a comparison of the TMB distribution across the CCA, cHCC-CCA, and HCC samples. TMB thresholds of 10 mut/Mb and 20 mut/Mb are labeled on the Y-axis. Pairwise comparisons of the median TMB were performed using the Wilcoxon rank sum test. The median TMB was comparable across all three diseases, CCA (2.5 mut/Mb), HCC (3.5 mut/Mb) and cHCC-CCA (2.6 mut/Mb). The prevalence of TMB-H (as defined by ≥20 mut/Mb), gLOH-H (as defined by ≥16%), and MIS-H across the CCA, cHCC-CCA, and HCC samples is provided in Table 3.

MSI status was determined by analyzing 114 intronic homopolymer repeat loci for length variability and MSI high (MSI-H), defined, for the purposes of this example, as described in Trabucco et al., A Novel Next-Generation Sequencing Approach to Detecting Microsatellite Instability and Pan-Tumor Characterization of 1000 Microsatellite Instability-High Cases in 67,000 Patient Samples, J. Molecular Diagnostics, vol. 21, no. 6, pp. 1053-1066 (2019). MSI was assessable for 4826/4975 CCA samples, 73/73 cHCC-CCA samples, and 1408/1470 HCC samples. MSI for the machine-learning model was defined as a categorical feature as an MSI status of high (MSI-H), stable (MSS), intermediate (MSI-I), or unknown (MSI-U).

gLOH was determined for 3428/4975 CCA samples, 58/73 cHCC-CCA samples, and 1116/1470 HCC samples. gLOH was encoded for the machine-learning models as a continuous numeric feature. FIG. 6 shows a comparison of the gLOH distribution across the CCA, cHCC-CCA, and HCC samples. A gLOH threshold of 16% gLOH was labeled on the Y-axis. Pairwise comparisons of median percentage gLOH were performed using the Wilcoxon rank sum test. 20.2% of CCA were gLOH-H, in contrast to 6.9% of HCC. Amongst cHCC-CCA, 13.8% were gLOH-H. Median gLOH of CCA (10.5%) was higher than that of HCC (5.4%, p<0.001) and comparable to cHCC-CCA (9.2%, p=0.04). However, genomic alterations in the homologous recombination repair pathway were seen in similar frequencies across all three diseases.

TABLE 3

| Characteristic | CCA | cHCC-CCA | HCC |
|---|---|---|---|
| TMB-H | 1.3% | 1.4% | 0.9% |
| | (62/4903) | (1/71) | (13/1454) |
| MSI-H | 1.1% | 0% | 0.2% |
| | (52/4826) | (0/73) | (3/1408) |
| gLOH-H | 20.2% | 13.8% | 6.9% |
| | (691/3428) | (8/58) | (77/1116) |

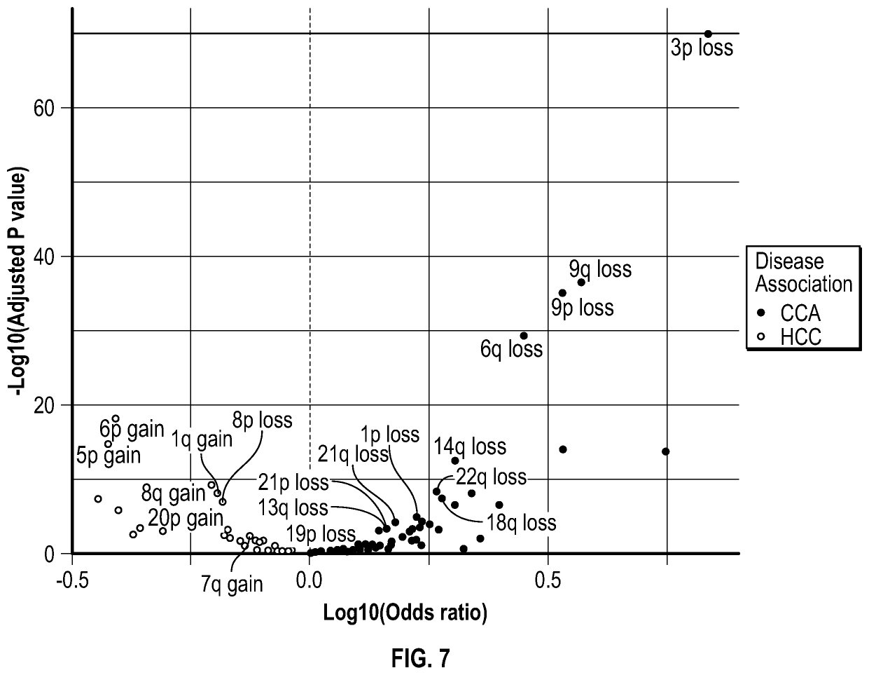

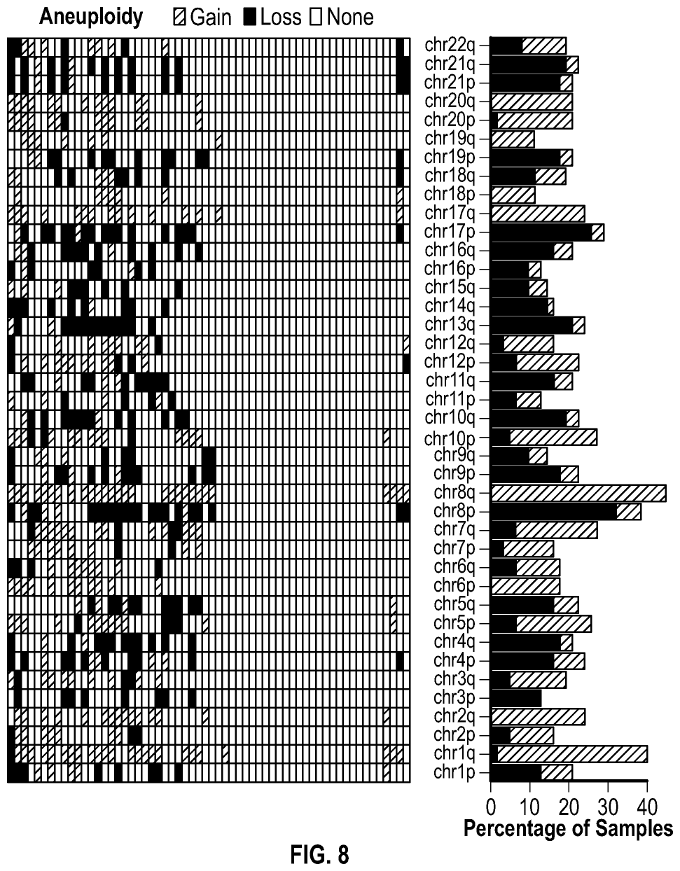

Within the baited regions of FoundationOne® solid tumor testing panel, chromosomal arm level aneuploidy was derived by comparing the log-ratio of read counts in tumor DNA to a process matched normal control and calculating signal to noise metrics, thereby measuring chromosome arm copy number. Based on the noise metrics in each sample, a per sample limit of detection was also calculated. A chromosome arm was considered lost or gained if >50% of the arm was altered. Chromosome arm level aneuploidy was assessable for all except for 4 acrocentric chromosomes, for which the p arm was excluded from analysis, for 4199/4975 CCA samples, 60/73 cHCC-CCA samples, and 1363/1470 HCC. Presence or absence of chromosome arm-level copy number gains and losses were encoded as a binary feature for the machine-learning model. FIG. 7 shows a volcano plot depicting the co-occurrence and mutual exclusivity of aneuploidy events between CCA and HCC. Chromosomal arm aneuploidies with a log 10 odds ratio greater than 0 are associated with CCA, and chromosomal arm aneuploidies having a log 10 odds ratio lower than 0 are associated with HCC. Only aneuploidy events with an adjusted P value≤0.01 and a prevalence≥10% in at least one disease are labelled. The two-tailed Fisher's exact test was used to evaluate the P values and odds ratios, which is used to determine associations between an event and disease. The Benjamini-Hochberg procedure was used to estimate the adjusted P values. The prevalence of various chromosomal arm aneuploidies identified in the CCA, cHCC-CCA and HCC samples are provided in Table 4 (chromosomal arm level gains) and Table 5 (chromosomal arm level losses). Each comparison listed in Table 4 and Table 5 was statistically significant, with an adjusted P value <0.05, as determined using a chi-square test for each of the 2×3 contingency tables and adjusted using the Benjamani-Hochberg procedure. FIG. 8 shows a landscape of the chromosomal aneuploidies detected across the cHCC-CCA samples, with the X-axis representing each cHCC-CCA sample and the Y-axis representing the assessed aneuploidy events. When CCA was compared with HCC, loss of 3p, 9p, 9q, 6q and gain of 6p, 5q were significantly enriched in CCA and HCC respectively. Within the cHCC-CCA population, the most frequent events were 8q gain (46.7%), 1q gain (40.0%), 8p loss (33.3%) and 17p loss (26.7%). In total, 39 unique chromosome arm level gains and 33 unique chromosome arm level losses were identified across 60 cases.

TABLE 4

| Chromosomal Arm | CCA | cHCC-CCA | HCC |
|---|---|---|---|
| Level Gain | (%) | (%) | (%) |
| 8q | 18.9 | 46.7 | 27.3 |
| 1q | 18.8 | 40.0 | 26.6 |
| 7q | 10.3 | 21.7 | 13.4 |
| 20p | 7.0 | 20.0 | 10.1 |
| 6p | 6.2 | 18.3 | 14.5 |
| 5q | 4.4 | 6.7 | 10.8 |

TABLE 5

| Chromosomal Arm | CCA | cHCC-CCA | HCC |
|---|---|---|---|
| Level Loss | (%) | (%) | (%) |
| 8p | 18.0 | 33.3 | 25.1 |
| 13q | 13.4 | 21.7 | 9.6 |
| 21q | 14.3 | 20.0 | 9.9 |
| 9p | 21.9 | 18.3 | 7.6 |
| 19p | 15.1 | 18.3 | 11.2 |
| 21p | 13.5 | 18.3 | 9.7 |
| 14q | 18.1 | 15.0 | 9.8 |
| 1p | 11.2 | 13.3 | 6.9 |
| 3p | 24.2 | 13.3 | 4.4 |
| 18q | 11.9 | 11.7 | 6.7 |
| 9q | 20.6 | 10.0 | 6.4 |
| 22q | 14.9 | 8.3 | 8.6 |
| 6q | 23.5 | 6.7 | 9.7 |

Genomic ancestry of patients was determined using a principal component analysis of genomic single nucleotide polymorphisms (SNPs) trained on data from the 1000 Genomes Project and each patient was classified as belonging to one of the following super populations: AFR (African), AMR (Ad Mixed American), EAS (East Asian), EUR (European) and SAS (South Asian). See Newberg et al., Abstract 1599: Determining patient ancestry based on targeted tumor comprehensive genomic profiling, Cancer Reesarch, vol. 79, no 13 Supplement (2019) and Carrot-Zhang, Comprehensive Analysis of Genetic Ancestry and its Molecular Correlates in Cancer, Cancer Cell, vol. 37, no. 5, pp. 639-654 (2020). For the machine-learning model, the genomic ancestry was encoded as a categorical feature.

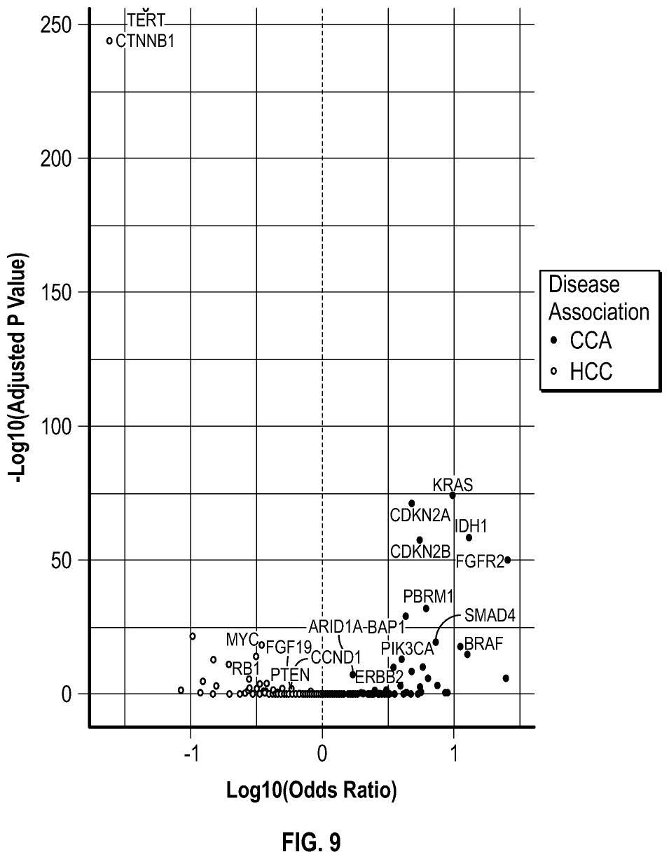

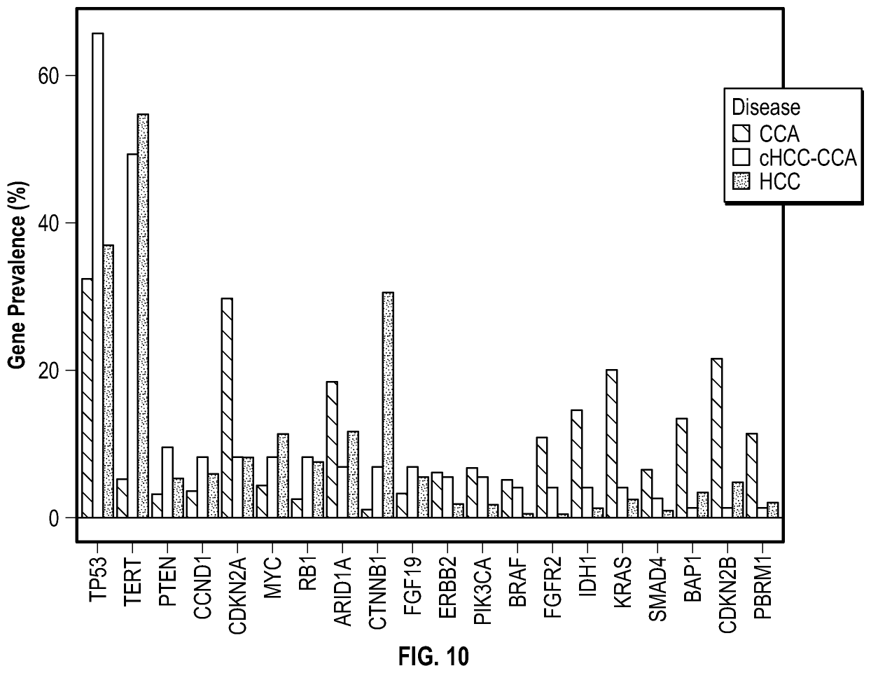

All genomic alteration (GA) prevalence reported in this example only include alterations described as functional/pathogenic in literature and seen in the Catalogue of Somatic Mutations in Cancer (COSMIC) repository (see Forbes et al., COSMIC: Somatic cancer genetics at high-resolution, Nucleic Acids Research, vol. 45, no. D1, pp. D777-D783 (2017)) or had a likely functional status (frameshift/truncation events in tumor suppressor genes). Variants of unknown significance were not studied. The set of functional variant features was derived from the cancer relevant genes commonly present across all FoundationOne® solid tumor testing panel versions. Within these 263 genes, presence or absence of a functional (known or likely functional) short variants, copy number alterations, or rearrangement in the gene. The presence or absence of a functional variant was encoded as a binary feature. FIG. 9 shows a volcano plot depicting the co-occurrence and mutual exclusivity of gene alterations between CCA and HCC. Only genes with an adjusted P value ≤0.05 and a prevalence ≥5% in either disease are labelled. A two-tailed Fisher's exact test was used to evaluate the P values and odds ratios that determines associations between genes and disease. The Benjamini-Hochberg procedure was used to estimate the adjusted P values. Genes with a log 10 odds ratio greater than 0 are associated with CCA, and genes having a log 10 odds ratio lower than 0 are associated with HCC. Prevalence of functional variants in select genes among the CCA, cHCC-CCA, and HCC samples are shown in FIG. 10 (for each gene, CCA, cHCC-CCA, and HCC are shown from left to right). When CCA was compared with HCC, genes were preferentially altered including ARID1A, BAP1, CDKN2A B, FGFR2, IDH1, KRAS, and PBRM1 in CCA, and CTNNB1, MYC, and TERT in HCC. Amongst cHCC-CCA cases, a median of 4 genomic alterations (GA) per tumor (range 0-14) was observed. Frequently altered genes in cHCC-CCA were TP53 (65.8%), TERT (49.3%) and PTEN (9.6%). Within this cohort, the most commonly altered genes with GA that are linked to benefit from targeted therapies were BRCA2 (8.2%, 67% short variant, 25% were biallelic losses; 33% rearrangements), ERBB2 (5.5%, 75% amplifications), IDH1 (4.1%, 100% R132), BRAF (4.1%, 100% V600E), FGFR2 (4.1%, 67% fusions), MET (2.7%, 100% amplifications), and accounted for 24.6% of cHCC-CCA.

Presence of HBV was determined by the identification of DNA sequences consistent with genomic HBV DNA. Sequencing reads left unmapped to the human reference genome (hg19) were de novo assembled by Velvet (see Zerbino, Using the Velvet de novo assembler for short-read sequencing technologies, Current Protocols in Bioinformatics, vol. 31, no. 1, pp. 11.5.1-11.5.12 (2010)) and the assembled contigs were competitively aligned by BLASTn21 to the NCBI database of over 3 million known viral nucleotide sequences. A positive viral status was determined by contigs at least 80 nucleotides in length and with at least 97% identity to the BLAST sequence. Genomic HBV was significantly associated with HCC compared to CCA (10.5% vs 1.9%, P-value=4.2e-42, Odds Ratio (OR)=6.14).

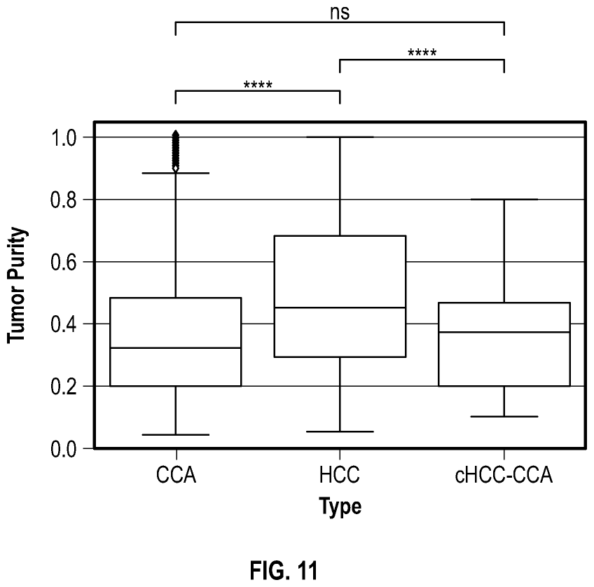

Tumor purity is a statistical quantification of the amount of tumor DNA component. This value was derived by simultaneous fitting segments of genomic allele counts and corresponding SNP frequencies to various statistical models, of which tumor purity is a modeling parameter. Tumor purity was added as a continuous numeric feature. FIG. 11 compares the computational tumor purity across CCA, HCC, and cHCC-CCA samples. The p values were estimated using a Wilcoxon rank sum test, with **** denoting a p-value <0.0001. The difference in tumor purity between CCA and HCC samples is statistically significant.

Additional clincopathological features were included in the study, including age of the subject at the time of profiling the cancer, the biological sex of the patient, the tissue biopsy site, and local/metastatic status of the tumor, each encoded as a categorical feature for the machine-learning model.

After filtering out for low prevalence and highly correlated features, a total of 157 genomically derived features (including 73 gene functional variant features, 78 chromosomal arm level aneuploidy events, TMB, tumor purity, genomic HBV status, genetic ancestry, gLOH, and MSI status) listed in Table 1 and 4 clinicopathological features (biological sex, age, local/metastatic status, and tumor biopsy site) were examined.

Machine-learning model.

To create a stringent high-quality training cohort of CCA and HCC samples, 2580/4975 CCA samples and 526/1470 HCC samples with either low tumor purity (less than 30%), poor sample quality (for example, the sample had significant contamination, the subject had a confirmed transplant, low sequencing coverage, low reference coverage, etc.), or copy number noise were filtered out. The resulting quality-controlled dataset of 2395 CCA samples and 944 HCC samples underwent an 80:20 class-weighted random split to yield 1916 CCA samples and 755 HCC samples for the training cohort and 479 CCA and 189 HCC cases for the testing cohort. A random forest-based machine-learning algorithm was trained using the 2671 training samples (1916 CCA training samples and 755 HCC training samples) using the genomic and clinicopathologic features. The trained model was then tested on the independent cohort of 668 cases (479 CCA samples and 189 HCC samples).

A binary classifier was built using the random forest algorithm using the 1916 CCA training samples and the 755 HCC training samples. The model parameters, including number of trees grown and size of the random feature subset considered at each split, were tuned by a cartesian hyperparameter grid search, to maximize AUC (ROC), with H2O. AI's scalable machine learning platform (v3.28.0.4) in R (v3.6.0). To adjust for class imbalance between CCA and HCC cases during model training, a stratified sampling methodology was used, and an equal number of cases were sampled from the CCA cases and HCC cases, equal to 80% of the total HCC cases in the training cohort. A genomics features only model (using the 157 genomically derived features) and a combined genomics and clinicopathologic features model (using the 157 genomically derived features and 4 clinicopathological features) were separately built and compared. The relative feature importance (by percentage) of a particular feature versus the entire set of 157 genomically derived features was determined for the genomic features only model, and the top 50 features and the relative importance are provided in FIG. 1. The top genomic features that the classifier used to distinguish CCA from HCC were (ranked in their descending order of their classification power): TERT variant, CTNNB1 variant, gLOH, =tumor purity, CDKN2A variant, chromosome 3p loss, CDKN2B variant, FGFR2 variant, IDH variant, TMB, KRAS variant, genomic ancestry, genomic HBV, chromosome 9q loss, and PBRM1 variant.

Age, biological sex, genomic HBV status, tumor site, and genomic ancestry of the CCA training samples and CCA testing samples are shown in Table 6.

TABLE 6

| | | CCA Training Samples | | CCA Testing Samples | |
|---|---|---|---|---|---|
| Feature | | (N = 1916, unless indicated) | | (N = 479, unless indicated) | |
| Age (Median, IQR) | | 63.0 | (55.0-70.0) | 62.0 | (54.0-69.5) |
| Biological Sex (Male) | | 886 | (46.2%) | 233 | (48.6%) |
| Genomic HBV | | 26 | (1.4%) | 10 | (2.1%) |
| Tumor Site (Local) | | 1567/1765 | (88.8%) | 395/443 | (89.2%) |
| Genomic | African | 121 | (6.3%) | 33 | (6.9%) |
| Ancestry | Ad Mixed | 236 | (12.3%) | 57 | (11.9%) |
| | American | | | | |
| | East Asian | 134 | (7.0%) | 41 | (8.6%) |
| | European | 1403 | (73.2%) | 341 | (71.2%) |
| | South Asian | 22 | (1.1%) | 7 | (1.5%) |

Age, biological sex, genomic HBV status, tumor site, and genomic ancestry of the HCC training samples and HCC testing samples are shown in Table 7.

TABLE 7

| | | HCC Training Samples | | HCC Testing Samples | |
|---|---|---|---|---|---|
| Feature | | (N = 755, unless indicated) | | (N = 189, unless indicated) | |
| Age (Median, IQR) | | 63.0 | (57.0-71.0) | 64.0 | (59.0-69.0) |
| Biological Sex (Male) | | 569 | (75.4%) | 136 | (72.0%) |
| Genomic HBV | | 95 | (12.6%) | 18 | (9.5%) |
| Tumor Biopsy Site (Local) | | 500/673 | (74.2%) | 123/173 | (71.1%) |
| Genomic | African | 99 | (13.1%) | 24 | (12.7%) |
| Ancestry | Ad Mixed | 106 | (14.0%) | 19 | (10.1%) |
| | American | | | | |
| | East Asian | 87 | (11.5%) | 23 | (12.2%) |
| | European | 445 | (58.9%) | 121 | (64.0%) |
| | South Asian | 18 | (2.4%) | 2 | (1.1%) |

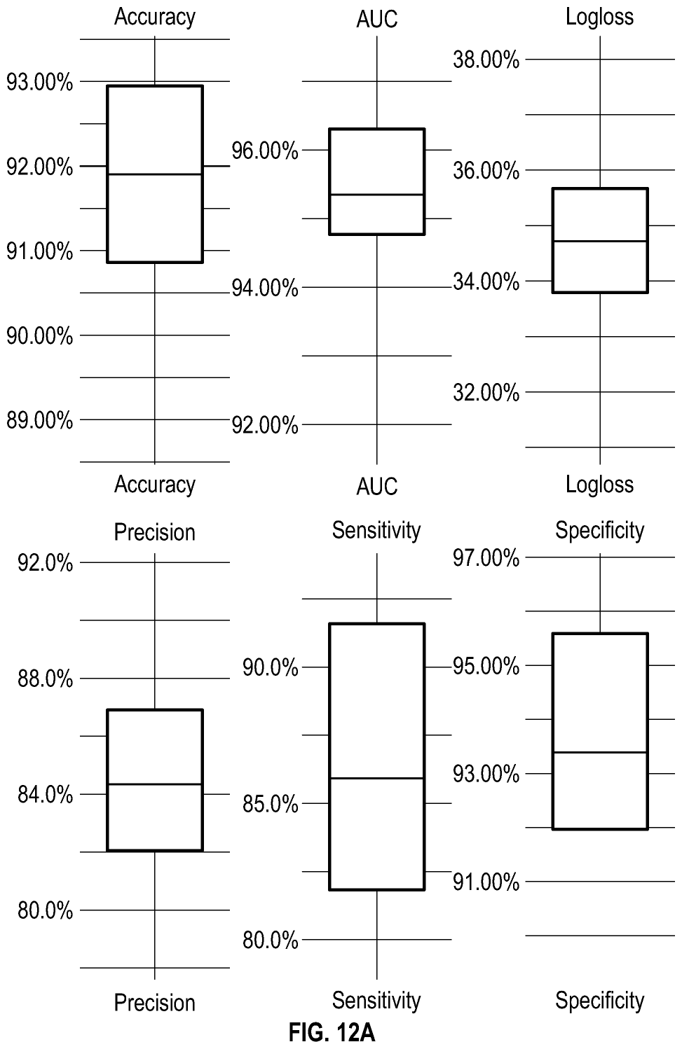

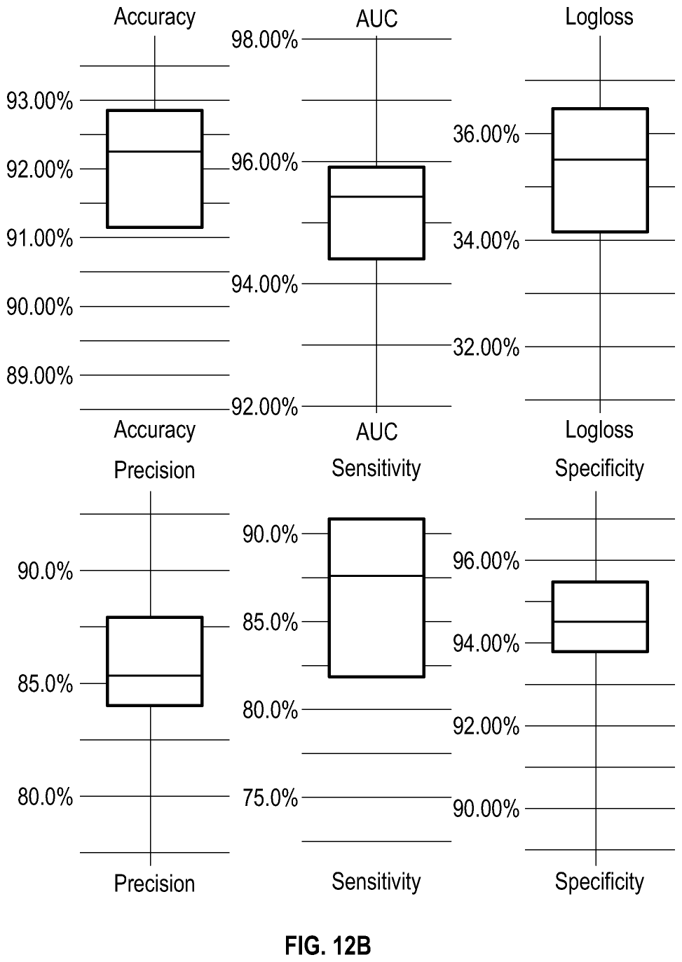

Prediction performance of the model was estimated on the training cohort by determining 10-fold cross-validation metrics including accuracy AUC, log loss, precision, sensitivity, and specificity. FIG. 12A provides AUC, log loss, precision, sensitivity, and specificity for the genomics features only model, and FIG. 12B provides AUC, log loss, precision, sensitivity, and specificity for the genomics and clinicopathologic features model. Based on 10-fold cross validation on the training dataset, the genomic features only model's mean sensitivity and specificity were 86.6% and 93.5%, respectively (median sensitivity and specificity were 85.9% and 93.4%, respectively). Based on 10-fold cross validation on the training dataset, the genomics and clinicopathologic features model's mean sensitivity and specificity were 85.2% and 94.4%, respectively (median sensitivity and specificity were 87.6% and 94.5% respectively).

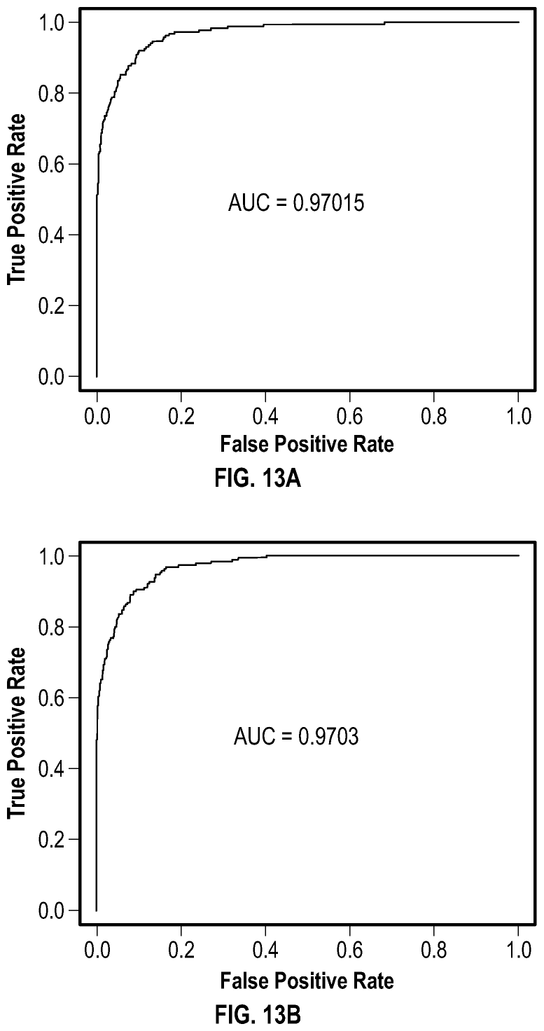

The independent test cohort of 479 CCA samples and 189 HCC samples was also used to evaluate the performance of classification. FIG. 13A shows an AUC (ROC) curve for the genomics features only model, and FIG. 13B shows an AUC (ROC) curve for the genomics features and clinicopathological features model. Both the genomic features only model and the genomic features and clinicopathological features model obtained a classification accuracy of 91% (95% confidence interval: 88.8-93.2) on the held-out testing dataset. Clinicopatholgoic features such as sex of the patient, biopsy, site of the patient's tumor specimen, and age of the patient at the time the sample was acquired were all found to be significantly associated with the presence or absence of genomic features, including but not limited to variants in TERT, CTNNB1, IDH1, and FGFR2, across HCC and CCA samples. See, for example Table 8, which shows the association of sex of the patient and the presence or absence of genomic features. An odds ratio greater than 1 denotes an association with the male sex and an odds ratio lesser than 1 denotes an association with the female sex. Table 9 shows the association of tissue biopsy site of the tumor specimen sent for comprehensive genomic profiling testing and the presence or absence of genomic features. An odds ratio greater than 1 denotes an association with non-liver biopsy and an odds ratio lesser than 1 denotes an association with liver biopsy. Table 10 shows the association of age of the patient at the time of comprehensive genomic profiling testing and presence or absence of genomic features. Age has been binarized into two groups, age above median (age>=median) and age below median (age median), median being 63 years. An odds ratio greater than 1 denotes an association with older age and an odds ratio lesser than 1 denotes an association with younger age. The Fisher's exact test was, used to estimate the odds ratio, P value and the Benjamani-Hochberg procedure was used to adjust the P values. Only features with an adjusted P value<=0.05 are shown in Tables 8-10.

TABLE 8

| Feature | Odds Ratio | P Value |
|---|---|---|
| CTNNB1 variant | 5.10565694 | 7.41E−34 |
| TERT variant | 4.7324465 | 1.97E−57 |
| HBV | 3.30982845 | 2.14E−10 |
| RBM10 variant | 2.59629227 | 0.00602137 |
| VEGFA variant | 2.40325252 | 0.00364196 |
| KMT2D variant | 1.91649701 | 0.00276261 |
| chr3p gain | 1.79969213 | 0.00840582 |
| chr5q gain | 1.5190417 | 0.00162391 |
| MYC variant | 1.45141615 | 0.0043765 |
| chr8q gain | 1.43993819 | 9.53E−06 |
| TP53 variant | 1.30038584 | 0.0003454 |
| CDKN2A variant | 0.81126109 | 0.00593256 |
| chr9p loss | 0.79582528 | 0.00501784 |
| chr14q loss | 0.76746778 | 0.00190257 |
| CDKN2B variant | 0.74318826 | 0.00031627 |
| chr6q loss | 0.73995614 | 0.00012847 |
| chr9q loss | 0.71432341 | 6.40E−05 |
| chr12q loss | 0.70363274 | 0.00564442 |
| chr11p loss | 0.69205965 | 0.00264584 |
| PBRM1 variant | 0.68621207 | 0.00151187 |
| chr3p loss | 0.66070833 | 2.11E−07 |
| BAP1 variant | 0.6388442 | 2.05E−05 |
| PTEN variant | 0.59932562 | 0.00265997 |
| BRAF variant | 0.59076015 | 0.00632729 |
| IDH2 variant | 0.55153321 | 0.00317068 |
| PIK3CA variant | 0.51978 | 2.12E−05 |
| IDH1 variant | 0.5019117 | 2.24E−10 |
| FGFR2 variant | 0.42749797 | 1.04E−13 |

TABLE 9

| Feature | Odds Ratio | P Value |
|---|---|---|
| NFE2L2 variant | 2.47529865 | 0.00233763 |
| CTNNB1 variant | 2.41484152 | 1.27E−12 |
| STK11 variant | 2.26445053 | 0.00011871 |
| TERT variant | 1.98424697 | 2.48E−12 |
| chr13q loss variant | 0.6492039 | 0.00023683 |
| chr14q loss variant | 0.61978614 | 1.01E−05 |
| chr9p loss variant | 0.60543021 | 1.21E−06 |
| chr2q gain | 0.59211503 | 0.00044292 |
| chr6q loss | 0.59148613 | 1.07E−07 |
| chr3p loss | 0.55592263 | 8.26E−09 |
| chr9q loss | 0.55535283 | 5.77E−08 |
| FGFR2 variant | 0.55176813 | 7.43E−05 |
| IDH2 variant | 0.37577585 | 0.00098868 |
| IDH1 variant | 0.33238521 | 2.31E−12 |
| ERRFI1 variant | 0.27046584 | 0.00483249 |

TABLE 10

| Feature | Odds Ratio | P Value |
|---|---|---|
| DNMT3A variant | 2.19963633 | 0.00091558 |
| chr17p gain | 2.16128241 | 0.00156008 |
| TERT variant | 1.63622145 | 1.98E−08 |
| CTNNB1 variant | 1.59593119 | 5.74E−05 |
| chr8p loss | 1.28822026 | 0.00122068 |
| FGFR2 variant | 0.47928873 | 1.91E−10 |
| HBV | 0.36434284 | 1.89E−08 |

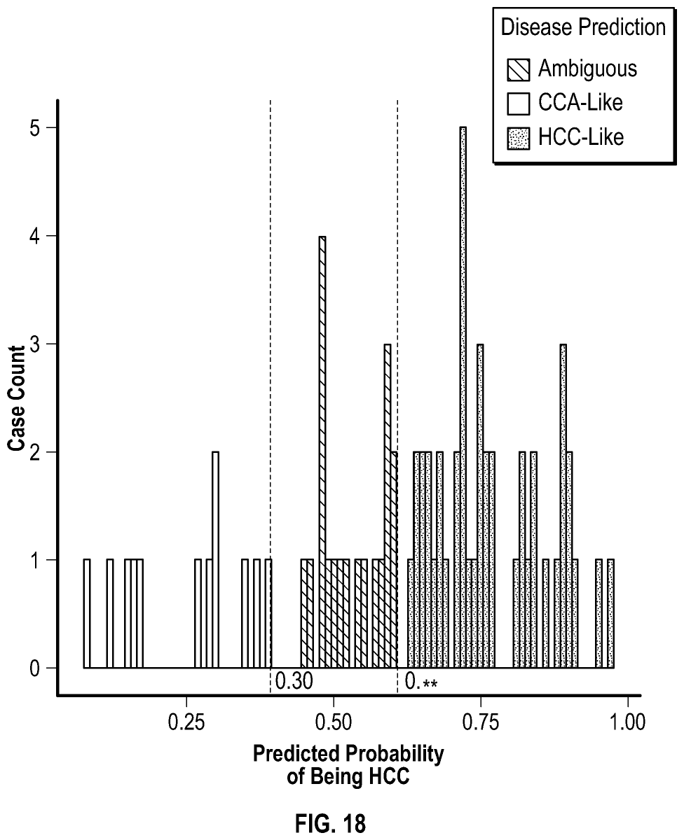

The genomics only model was subsequently applied on the cHCC-CCA cohort to classify each cHCC-CCA case as CCA-like or HCC-like or ambiguous. A probability threshold that maximized the Matthew's correlation coefficient (MCC; 0.613 for this application) for the training cohort was used as the threshold to annotate cHCC-CCA cases as CCA-like and HCC-like. cHCC-CCA cases with a predicted probability less that this value were annotated as ambiguous. When applied to the cHCC-CCA cohort, 16.4% (12/73) of the samples were classified as CCA-like and 575% (42/73) classified as HCC-like. Thus, the model classified over 70% (i.e., 74%, 54/73) of cHCC-CCA as CCA-like or HCC-like on the basis of genomic profiles generated during regular clinical management. The remaining 26.3% (19/73) of the cHCC-CCA cases were classified as ambiguous. See FIG. 18.

Using the model, test cHCC-CCA sample with the presence of IDH1 functional variant (known or likely functional variant), presence of ARID1A functional variant, absence of genomic HBV, absence of TERT functional variant, absence of CTNNB1 functional variant, absence of FGFR2 functional variant, European ancestry, TMB of 0 mutations/Mb, and gLOH of 12.51% amongst other genomic features, would be assigned a CCA probability of 0.88 and an HCC probability of 0.12, and subsequently classified as a CCA-like cHCC-CCA. Another test cHCC-CCA sample with the presence of TERT functional variant, presence of CTNNB1 functional variant, absence of genomic HBV, gLOH of 6.47%, TMB of 4.4 mutations/Mb, absence of FGFR2 functional variant, absence of IDH1 functional variant, amongst other genomic features, would be assigned a CCA probability of 0.03 and an HCC probability of 0.97 and subsequently classified as an HCC-like cHCC-CCA.

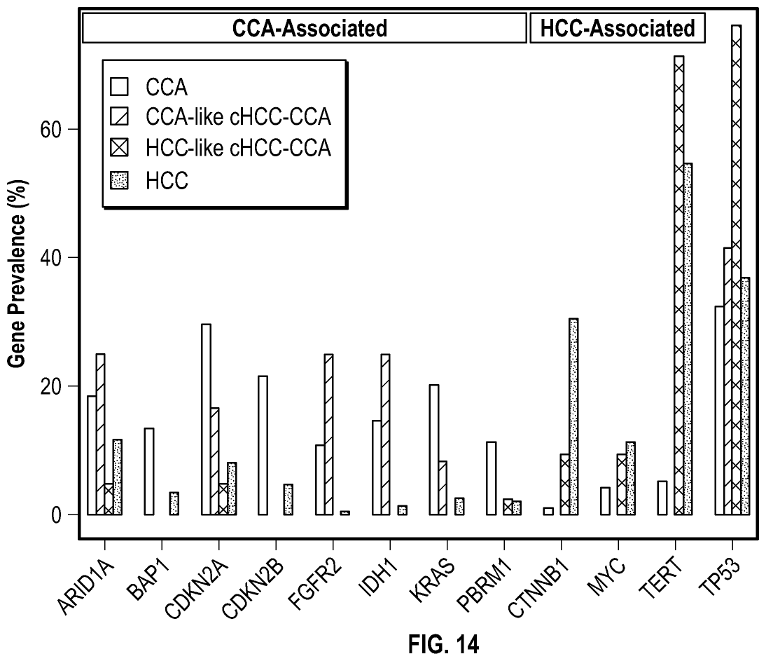

The presence of a functional variant in certain genes in CCA, model-classified CCA-like cHCC-CCA, model-classified HCC-like cHCC-CCA, and HCC was compared, as shown in FIG. 14 (shown, for each gene from left to right, CCA, model-classified CCA-like cHCC-CCA, model-classified HCC-like cHCC-CCA, and HCC). It was found that functional variants in ARID1A, BAP1, CDKN2A, CDKN2B, FGFR2, IDH1, KRAS, and PBRM1 tended to be associated with CCA, whereas functional variants in CTNNB1, MC, and TERT tended to be associated with HCC. Additional characteristics for the model classified CCA-like cHCC-CCA and HCC-like cHCC-CCA samples is provide in Table 11.

TABLE 11

| | | CCA-like | HCC-like |
|---|---|---|---|
| | | cHCC-CCA | cHCC-CCA |
| Feature | | (N = 12) | (N = 42) |
| Genomic | African | 0% | 19% |
| Ancestry | East Asian | 8.3% | 19% |
| | European | 75% | 47.6% |
| Chromosomal | 3p loss | 25% | 7.1% |
| Arm | 6q loss | 9.1% | 0% |
| Aneuploidy | 9p loss | 16.7% | 14.3% |
| | 9q loss | 8.3% | 7.1% |

| Genomic HBV | 0% | | 16.7% | |
|---|---|---|---|---|
| Median TMB (mut/Mb, IQR) | 1.1 | (0-2.5) | 3.6 | (2.5-5.9) |
| Median gLOH (%, IQR) | 12.4 | (10.4-15.0) | 8.7 | (5.4-11.9) |
| Tumor Purity (%, IQR) | 30.0 | (21.5-39.3) | 37.0 | (23.3-48.0) |

The 19 ambiguous cHCC-CCA cases harbored genomic features associated with both CCA and HCC. Notable examples include a case with presence of genomic HBV, wildtype FGFR2, and wildtype IDH1, all of which resemble HCC but also harbored a gLOH of 10.6% and 3p loss which is more CCA-like. Another case harbors a genomic alteration in TERT, a TMB of 2.5 mut/Mb, gLOH of 4%, wildtype FGFR2, and wildtype IDH1, all consistent with HCC, but also an ERBB2 alteration, the latter being frequently associated with biliary tract cancer. Almost half of these ambiguous cases (8/19) had lower tumor purity, which is important as tumor purity is a strong feature differentiating CCA and HCC in this series. A low tumor purity reduces the sensitivity of the genomic profile test to detect low allele frequency genomic alterations, hence affecting classification accuracy.

### Example 2

67 cHCC-CCA (N=40 HCC-like, N=10 CCA-like, and N=17 ambiguous, as determined by a random forest-based model trained on HCC and CCA training sample data sets), 1310 HCC samples, and 4409 CCA samples were evaluated for cancer cell fraction (CCF) of their short variant somatic variants. CCF was estimated using a publicly available tool called PyClone25 v0.13.1 using default parameters. Somatic variants were distinguished from germline variants using the method described in Sun et al., A computational approach to distinguish somatic vs. germline origin of genomic alterations from deep sequencing of cancer specimens without a matched normal, PLoS Computational. Biology, vol. 14, no. 2, e1005965 (2018).

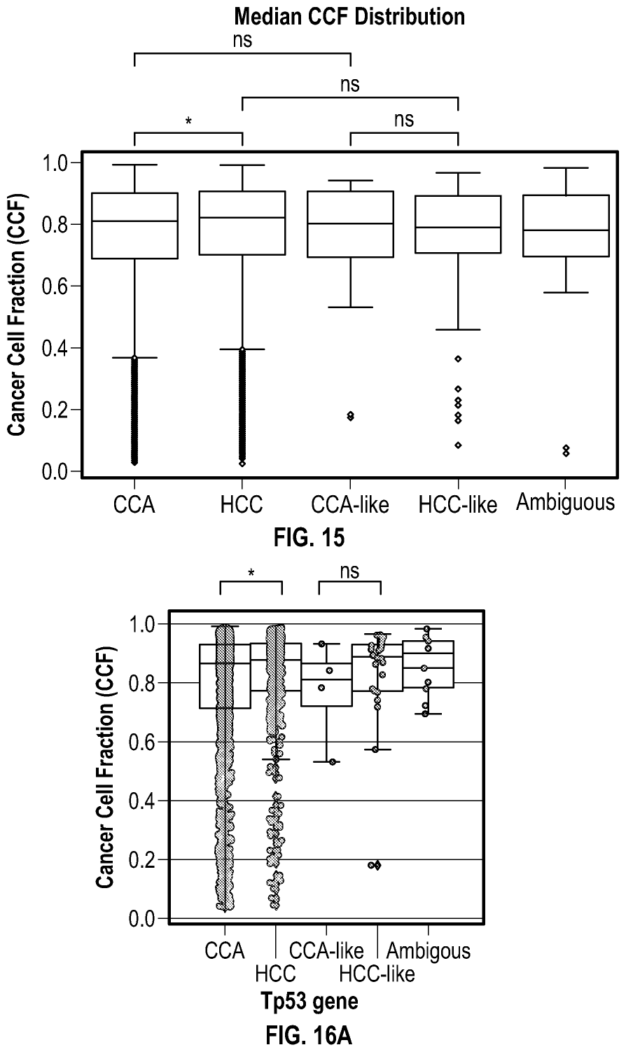

Across all short somatic variants in all interrogated genes in each disease, CCA had a median CCF of 0.81 [Inter quantile range (IQR): 0.69-0.90], HCC had a median CCF of 0.82 [IQR: 0.70-0.90], and cHCC-CCA had a median CCF of 0.78 [IQR: 0.70-0.89]. There was no significant difference in CCF amongst the cHCC-CCA cases when broken down by CCA-like, HCC-like, and ambiguous (FIG. 15), although the ambiguous cHCC-CCA samples had the lowest CCF amongst all the groups.

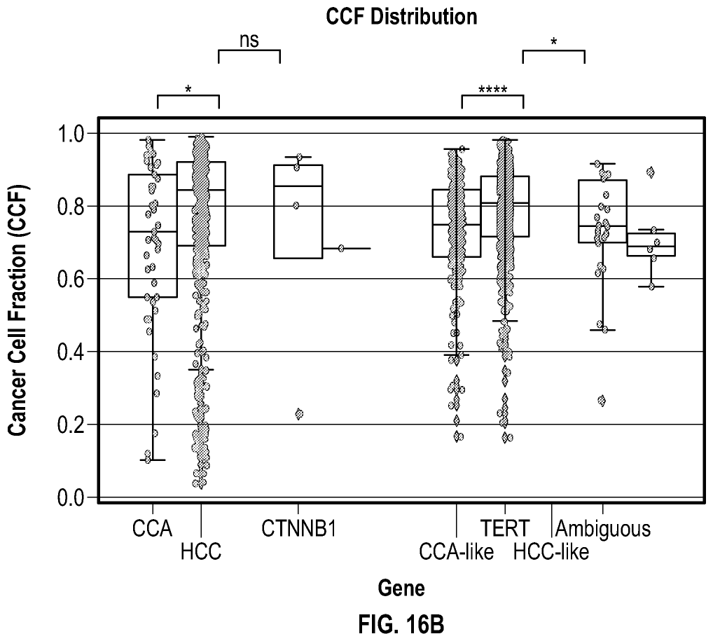

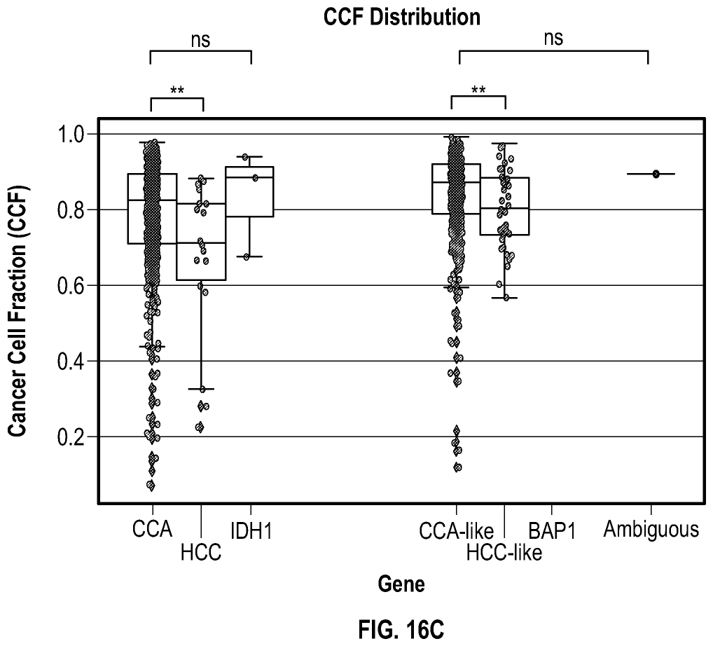

The CCF of short somatic variants in genes previously established as CCA or HCC associated were further evaluated. In addition to the CCA and HCC associated genes, short somatic variants in TP53 were also evaluated because TP53 has been previously implicated in liver cell plasticity. 17 genes (TP53, TERT, CTNNB1, PTEN, IDH1, PBRM1, BAP1, KRAS, FGFR2, SMAD4, PIK3CA, ERBB2, CDKN2A, CDKN2B, ARID1A, CCND1 and BRAF) were evaluated to determine whether there were differences in their CCF between HCC and CCA specimens. A significant difference between the CCF in CCA specimens and the CCF in HCC specimens was detected for 5 of the 17 genes: TP53, TERT, CTNNB1, IDH1, BAP1. See Table 12 and FIGS. 16A-16C (**** indicates p-value <0.0001; *** indicates p-value 0.0001 to 0.001; ** indicates p-value 0.001 to 0.01; * indicates p-value 0.01 to 0.05; ns indicates p-value >0.05).

TABLE 12

| | | | Median | 1st | 3rd |
|---|---|---|---|---|---|
| Gene | Type | N | CCF | Quantile | Quantile |
| BAP1 | CCA | 488 | 0.874 | 0.791 | 0.921 |
| | HCC | 41 | 0.803 | 0.734 | 0.886 |
| | ambiguous | 1 | 0.895 | 0.895 | 0.895 |
| CTNNB1 | CCA | 49 | 0.729 | 0.550 | 0.886 |
| | HCC | 458 | 0.844 | 0.691 | 0.920 |
| | HCC-like | 4 | 0.854 | 0.658 | 0.914 |
| | Ambiguous | 1 | 0.683 | 0.683 | 0.683 |
| IDH1 | CCA | 715 | 0.826 | 0.712 | 0.896 |
| | CCA-like | 3 | 0.885 | 0.782 | 0.913 |
| | HCC | 18 | 0.713 | 06.15 | 0.816 |
| TERT | CCA | 252 | 0.749 | 0.661 | 0.845 |
| | HCC | 776 | 0.810 | 0.717 | 0.882 |
| | HCC-like | 29 | 0.746 | 0.699 | 0.873 |
| | Ambiguous | 6 | 0.690 | 0.664 | 0.726 |
| TP53 | CCA | 1575 | 0.866 | 0.714 | 0.928 |
| | CCA-like | 4 | 0.812 | 0.720 | 0.864 |
| | HCC | 529 | 0.878 | 0.773 | 0.933 |
| | HCC-like | 28 | 0.888 | 0.770 | 0.928 |
| | Ambiguous | 9 | 0.850 | 0.770 | 0.941 |

For TERT, the HCC samples had a median CCF at 0.81 [IQR: 0.72-0.88] and the CCA samples had a median CCF of 0.75 [IQR 0.66-0.85]. For CTNNB1, the HCC-like cHCC-CCA samples had a median CCF of 0.85 [IQR: 0.66-0.91] and the CCA samples had a median CCF of 0.72 [IQR 0.55-0.89]. For IDH1, the HCC samples had a median CCF of 0.71 [IQR 0.61-0.82] and the CCA samples had a median CCF of 0.83 [IQR 0.71-0.90]. For BAP1 the HCC samples had a median CCF of 0.80 [IQR 0.73-0.89] and the CCA samples have a median CCF of 0.87 [IQR 0.79-0.92]. In summary, the CCF of CCA-associated genes (IDH1 and BAP1) was higher in CCA samples than the HCC samples and the CCF of HCC-associated genes (TERT and CTNNB1) was higher in HCC samples than the CCA samples.

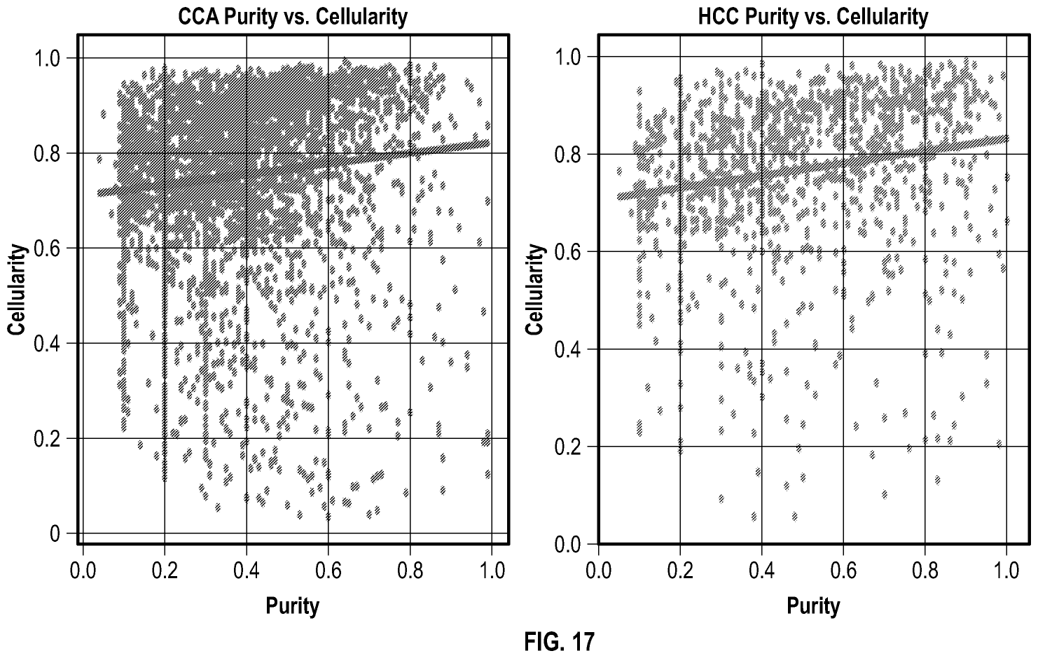

Contamination of normal cells in tumor specimens as a potential confounding factor of CCF estimation was also investigated. The three different tumor types CCA and HCC display a significant difference in their tumor purity (FIG. 11), and the independence of CCF was evaluated by determining the correlation between tumor purity and estimated CCF (FIG. 17). The observed Spearman correlation factors were 0.11 and 0.12 for CCA samples and HCC samples (p-value <0.001), respectively, indicating weak correlation between tumor purity and CCF. Additionally, since the tumor purity of HCC samples is significantly higher than CCA samples (p<0.0001; FIG. 11), the difference in overall distribution seen in FIG. 17 may be due to the difference in sample purity.

Variants in CCA-associated genes such as IDH1 are often found in HCC samples, and variants in HCC-associated genes such as TERT are often found in CCA samples. At a population level, however, the variants in IDH1 are more clonal in CCA than HCC, and variants in TERT are more clonal in HCC than CCA, even though the CCF across all short somatic variants in HCC and CCA are similar. Thus, not only the presence/absence of variants in certain genes, but also the cancer cell fraction of these alterations, may be useful in characterizing cHCC-CCA as CCA-like or HCC-like. Given these observations, the machine-learning model trained on HCC and CCA samples, to classify a cHCC-CCA case as CCA-like or HCC-like, can incorporate CCF as an additional feature.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1. A method comprising:
generating genomic data for a sample from a subject having cancer, comprising:
providing a plurality of nucleic acid molecules obtained from the sample;
ligating one or more adapters to one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
analyzing, by one or more processors, the plurality of sequence reads to generate the test genomic data;
receiving, at one or more of the one or more processors, test data for the sample, wherein the test data comprises the genomic data for the sample;
inputting, using the at least one processor, the test data into a combined hepatocellular cholangiocarcinoma (cHCC-CCA) machine-learning model trained using hepatocellular carcinoma (HCC) data comprising HCC genomic data from a plurality of HCC samples and cholangiocarcinoma (CCA) data comprising CCA genomic data from a plurality of CCA samples, wherein the cHCC-CCA machine-learning model is configured to classify the sample, based on the test data, as CCA-like, or HCC-like, or ambiguous; and
classifying, by the at least one processor using the cHCC-CCA machine-learning model, the sample as HCC-like, or CCA-like, or ambiguous.

2-8. (canceled)

9. A method, comprising:
receiving, at one or more processors, test data comprising genomic data for a sample from a subject having cancer;
inputting, using the one or more processors, the test data into a combined hepatocellular cholangiocarcinoma (cHCC-CCA) machine-learning model trained using hepatocellular carcinoma (HCC) data comprising HCC genomic data from a plurality of HCC samples and cholangiocarcinoma (CCA) data comprising CCA genomic data from a plurality of CCA samples, wherein the cHCC-CCA machine-learning model is configured to classify the sample, based on the test data, as CCA-like, HCC-like, or ambiguous; and
classifying, using the one or more processors and the cHCC-CCA machine-learning model, the sample as HCC-like, CCA-like, or ambiguous.

10. The method of claim 9, wherein the cHCC-CCA machine-learning model is a probabilistic classifier configured to compute a probability that the sample is HCC-like or a probability that the sample is CCA-like.

11. The method of claim 9, further comprising training the cHCC-CCA machine learning model using the HCC data and the CCA data.

12. The method of claim 9, wherein the sample is a bile duct cancer sample.

13. (canceled)

14. The method of claim 9, wherein the sample is a combined hepatocellular cholangiocarcinoma (cHCC-CCA) sample.

15-17. (canceled)

18. The method of claim 9, wherein the genomic data for the sample, the HCC genomic data, and the CCA genomic data each comprise a tumor purity.

19. The method of claim 9, wherein the genomic data for the sample, the HCC genomic data, and the CCA genomic data each comprises a chromosomal aneuploidy status for one or more chromosomes or chromosome arms.

20. (canceled)

21. The method of claim 9, wherein the genomic data for the sample, the HCC genomic data, and the CCA genomic data each comprise a cancer cell fraction (CCF) for one or more genes, wherein the CCF for the one or more genes is differentially represented in CCA and HCC.

22. (canceled)

23. The method of claim 9, wherein the genomic data for the sample, the HCC genomic data, and the CCA genomic data each comprise a functional variant status for each of one or more genes.

24-27. (canceled)

28. The method of claim 9, wherein the genomic data for the sample, the HCC genomic data, and the CCA genomic data each comprises a tumor mutational burden (TMB).

29-30. (canceled)

31. The method of claim 9, wherein the genomic data for the sample, the HCC genomic data, and the CCA genomic data each comprises a microsatellite instability (MSI) status.

32. (canceled)

33. The method of claim 9, wherein genomic data for the sample, the HCC genomic data, and the CCA genomic data each comprises a genome-wide loss of heterozygosity (gLOH) status.

34-35. (canceled)

36. The method of claim 9, wherein the test data, the HCC data, and the CCA data each comprises an ancestry status.

37-38. (canceled)

39. The method of claim 9, wherein the test data, the HCC data, and the CCA data each comprise a hepatitis B virus (HBV) status.

40. (canceled)

41. The method of claim 9, wherein the test data, the HCC data, and the CCA data each further comprises one or more clinicopathological features.

42. (canceled)

43. The method of claim 9, wherein the genomic data for the sample, the HCC genomic data, and the CCA genomic data are each determined from sequencing data.

44-63. (canceled)

64. A method of selecting a treatment for a cancer in a subject, comprising:
obtaining a classification of a sample associated with the cancer as HCC-like or CCA-like, wherein the sample was classified using the method of claim 9; and
selecting the treatment for the cancer, wherein the treatment is selected to effectively treat HCC if the sample is classified as HCC-like, and the treatment is selected to effectively treat CCA if the sample is classified as CCA-like.

65. The method of claim 64, further comprising administering the selected treatment to the subject.

66. A method of treating a cancer in a subject, comprising:
obtaining a classification of a sample from the subject as HCC-like or CCA-like, wherein the sample was classified using the method of claim 9; and
administering a treatment to the subject, wherein the treatment is selected to effectively treat HCC if the sample is classified as HCC-like, and the treatment is selected to effectively treat CCA if the sample is classified as CCA-like.

67. The method of any one of claims 64-66, wherein the sample is classified as HCC-like, and the treatment comprises a localized therapy, a multi-targeted tyrosine kinase inhibitor, or an immunotherapy.

68. The method of claim 67, wherein the treatment comprises a multi-targeted tyrosine kinase inhibitor.

69. The method of claim 68, wherein the multi-targeted tyrosine kinase inhibitor comprises axitinib, brivanib, cabozantinib, cediranib, donofenib, dovitinib, lenvatinib, linifanib, nintedanib, regorafenib, sorafenib, or sunitinib.

70. The method of claim 67, wherein the treatment comprises an immunotherapy.

71. The method of claim 70, wherein the immunotherapy comprises an immune checkpoint inhibitor.

72. The method of claim 71, wherein the immune checkpoint inhibitor is tremelimumab, ipilimumab, nivolumab, pembrolizumab, camrelizumab, tislelizumab, avelumab, atezolizumab, or durvalumab.

73. The method of any one of claims 64-66, wherein the cancer is classified as CCA-like, and the treatment comprises a chemotherapy or a targeted therapy.

74. The method of claim 73, wherein the treatment comprises a chemotherapy.

75. The method of claim 74, wherein the chemotherapy comprises a fluoropyrimidine, a platinum agent, or a taxane.

76. The method of claim 75, wherein the chemotherapy comprises gemcitabine, capecitabine, doxifluridine, fluorouracil, irinotecan, tegafur, cisplatin, oxaliplatin, docetaxel, or paclitaxel.

77. The method of claim 73, wherein the treatment comprises a targeted therapy.

78. The method of claim 77, wherein the targeted therapy comprises a kinase-specific inhibitor.

79. The method of claim 77, wherein the treatment comprises an IDH1 inhibitor, an FGFR2 inhibitor, a MEK inhibitor, or an mTOR inhibitor.

80. The method of claim 79, wherein the treatment comprises an IDH1 inhibitor, wherein the cancer has an IDH1 mutation.

81. The method of claim 79 or 80, wherein the treatment comprises an IDH1 inhibitor, and wherein the IDH1 inhibitor is ivosidenib.

82. The method of claim 79, wherein the treatment comprises an FGFR2 inhibitor, wherein the cancer has a FGFR2 mutation.

83. The method of claim 79 or 82, wherein the treatment comprises an FGFR2 inhibitor, and the FGFR2 inhibitor is pemigatinib, infigratinib, derazantinib, or bemarituzumab.

84. The method of claim 79, wherein the treatment comprises an MEK inhibitor or an mTOR inhibitor, wherein the cancer has a KRAS mutation.

85. The method of claim 79 or 84, wherein the treatment comprises an MEK inhibitor, and wherein the MEK inhibitor is selumetinib.

86. The method of claim 79 or 84, wherein the treatment comprises an mTOR inhibitor, and wherein the mTOR inhibitor is everolimus.

87. The method of any one of claims 9-86, comprising sequencing nucleic acid molecules from the sample to obtain at least a portion of the genomic data for the sample.

88. A system, comprising:
one or more processors;
a memory; and
one or more programs, wherein the one or more programs are stored in the memory and configured to be executed by the one or more processors, the one or more programs including instructions for implementing a method, comprising:
receiving, at the one or more processors, test data comprising genomic data for a sample from a subject having cancer;
inputting, using the one or more processors, the test data into a combined hepatocellular cholangiocarcinoma (cHCC-CCA) machine-learning model trained using hepatocellular carcinoma (HCC) data comprising HCC genomic data from a plurality of HCC samples and cholangiocarcinoma (CCA) data comprising CCA genomic data from a plurality of CCA samples, wherein the cHCC-CCA machine-learning model is configured to classify the sample, based on the test data, as CCA-like, HCC-like, or ambiguous; and
classifying, using the one or more processors and the cHCC-CCA machine-learning model, the sample as HCC-like, CCA-like, or ambiguous.

89. The system of claim 88, comprising a sequencer configured to sequence nucleic acids derived from cancer test sample.

90. The system of claim 88 or 89, wherein the cHCC-CCA machine-learning model is a probabilistic classifier configured to compute a probability that the sample is HCC-like or a probability that the cancer test sample is CCA-like.

91. The system of any one of claims 88-90, wherein the one or more programs further include instructions for training the cHCC-CCA machine learning model using the HCC data and the CCA data.

92. The system of any one of claims 88-81, wherein the sample is a bile duct cancer sample.

93. The system of claim 92, wherein the bile duct cancer sample is an intrahepatic bile duct cancer sample, an extrahepatic bile duct cancer sample, a perihilar bile duct cancer sample, or a distal bile duct cancer sample.

94. The system of any one of claims 88-93, wherein the cancer sample is a combined hepatocellular cholangiocarcinoma (cHCC-CCA) sample.

95. The system of any one of claims 88-91, wherein the cancer is a bile duct cancer.

96. The system of claim 95, wherein the bile duct cancer is an intrahepatic bile duct cancer, an extrahepatic bile duct cancer, a perihilar bile duct cancer, or a distal bile duct cancer.

97. The system of any one of claims 88-91, 95, and 96, wherein the cancer is a combined hepatocellular cholangiocarcinoma (cHCC-CCA).

98. The system of any one of claims 88-97, wherein the genomic data for the sample, the HCC genomic data, and the CCA genomic data each comprise a tumor purity.

99. The system of any one of claims 88-98, wherein the genomic data for the sample, the HCC genomic data, and the CCA genomic data each comprises a chromosomal aneuploidy status for one or more chromosomes or chromosome arms.

100. The system of claim 99, wherein the chromosomal aneuploidy status comprises a loss status or a gain status of one or more of a 1q arm, 2q arm, 5p arm, 6p arm, 6q arm, 7q arm, 8p arm, 8q arm, 10q arm, 17p arm, 17q arm, 18q arm, 20p arm, 20q arm, 21p arm, and 22q arm.

101. The system of any one of claims 88-100, wherein the genomic data for the sample, the HCC genomic data, and the CCA genomic data each comprise a cancer cell fraction (CCF) for one or more genes, wherein the CCF for the one or more genes is differentially represented in CCA and HCC.

102. The system of claim 101, wherein the CCF for one or more genes differentially represented in CCA and HCC comprises a CCF of one or more of TP53, CTNNB1, TERT, IDH1, and BAP1.

103. The system of any one of claims 88-102, wherein the genomic data for the sample, the HCC genomic data, and the CCA genomic data each comprise a functional variant status for each of one or more genes.

104. The system of claim 103, wherein the functional variant status is a presence or an absence of the functional variant for the gene.

105. The system of claim 103 or 104, wherein the functional variant caused by a single nucleotide variant (SNV), a multiple nucleotide variant (MNV), a copy number alteration, an indel, or a rearrangement.

106. The system of any one of claims 103-105, wherein the one or more genes comprises ARID1A, BAP1, BRAF, CCND1, CDKN2A, CDKN2B, CTNNB1, ERBB2, FGFR2, IDH1,KRAS, MTAP, PBRM1, PIK3CA, PTEN, MYC, RB1, SMAD4, or TERT.

107. The system of any one of claims 103-106, wherein the one or more genes comprises ARID1A, BAP1, CDKN2A, CDKN2B, CTNNB1, FGFR2, IDH1, KRAS, PBRM1, MYC, or TERT.

108. The system of any one of claims 88-107, wherein the test genomic data, the HCC genomic data, and the CCA genomic data each comprises a tumor mutational burden (TMB).

109. The system of claim 108, wherein the TMB is a continuous numeric feature.

110. The system of claim 108, wherein the TMB is a categorical feature.

111. The system of any one of claims 88-110, wherein the genomic data for the sample, the HCC genomic data, and the CCA genomic data each comprises a microsatellite instability (MSI) status.

112. The system of claim 111, wherein the MSI status is a categorical feature.

113. The system of any one of claims 88-112, wherein genomic data for the sample, the HCC genomic data, and the CCA genomic data each comprises a genome-wide loss of heterozygosity (gLOH) status.

114. The system of claim 113, wherein the gLOH status is a continuous numeric feature.

115. The system of claim 113, wherein the gLOH status is a categorical feature.

116. The system of any one of claims 88-115, herein the test data, the HCC data, and the CCA data each comprises an ancestry status.

117. The system of claim 116, wherein the ancestry status is a genomic ancestry status.

118. The system of claim 117, wherein the genomic ancestry status is a categorical feature, wherein the categorical feature is at least one of African, Ad Mixed American, East Asian, European, or South Asian.

119. The system of any one of claims 88-118, wherein the test data, the HCC data, and the CCA data each comprise a hepatitis B virus (HBV) status.

120. The system of claim 119, wherein the HBV status is determined by detecting a presence or absence of genomic HBV DNA.

121. The system of any one of claims 88-120, wherein the test data, the HCC data, and the CCA data each further comprises one or more clinicopathological features.

132. The system of any one of claims 88-131, wherein the cancer test sample is a solid tissue biopsy sample.

133. The system of claim 132, wherein the solid tissue biopsy sample is a formalin-fixed paraffin-embedded (FFPE) sample.

134. The system of any one of claims 88-131, wherein the cancer test sample is a liquid biopsy sample comprising circulating tumor DNA (ctDNA).

135. The system of any one of claims 88-131, wherein the sample is a liquid biopsy sample comprising circulating tumor cells (CTCs).

136. The system of claim 134 or 135, wherein the sample is a liquid biopsy sample and comprises blood, plasma, cerebrospinal fluid, sputum, stool, urine, or saliva.

137. The system of any one of claims 88-136, wherein the one or more programs further include instructions for generating a report identifying the cancer test sample as HCC-like, CCA-like, or ambiguous.

138. The system of claim 137, wherein the one or more programs further include instructions for displaying the report on an electronic display.

139. The system of claim 137 or 138, wherein the one or more programs further include instructions for transmitting the report to the subject or a healthcare provider for the subject.

140. The system of claim 139, wherein the report is transmitted via a computer network or a peer-to-peer connection.

141. The system of claim 139 or 140, wherein the report is an electronic medical record.

142. A non-transitory computer-readable storage medium storing one or more programs, the one or more programs comprising instructions, which when executed by one or more processors of an electronic device, cause the electronic device to implement a method, comprising:
receiving, at the one or more processors, test data comprising genomic data for a sample from a subject with cancer;
inputting, using the one or more processors, the test data into a combined hepatocellular cholangiocarcinoma (cHCC-CCA) machine-learning model trained using hepatocellular carcinoma (HCC) data comprising HCC genomic data from a plurality of HCC samples and cholangiocarcinoma (CCA) data comprising CCA genomic data from a plurality of CCA samples, wherein the cHCC-CCA machine-learning model is configured to classify the sample, based on the test data, as CCA-like, HCC-like, or ambiguous; and
classifying, using the one or more processors and the cHCC-CCA machine-learning model, the sample as HCC-like, CCA-like, or ambiguous.

143. The non-transitory computer-readable storage medium of claim 142, wherein the cHCC-CCA machine-learning model is a probabilistic classifier configured to compute a probability that the cancer test sample is HCC-like or a probability that the cancer test sample is CCA-like.

144. The non-transitory computer-readable storage medium of claim 142 or 143, wherein the one or more programs further include instructions, which when executed by one or more processors of an electronic device, cause the electronic device to train the cHCC-CCA machine learning model using the HCC data and the CCA data.

145. The non-transitory computer-readable storage medium of any one of claims 142-144, wherein the sample is a bile duct cancer sample.

146. The non-transitory computer-readable storage medium of claim 145, wherein the bile duct cancer sample is an intrahepatic bile duct cancer sample, an extrahepatic bile duct cancer sample, a perihilar bile duct cancer sample, or a distal bile duct cancer sample.

147. The non-transitory computer-readable storage medium of any one of claims 142-146, wherein the sample is a combined hepatocellular cholangiocarcinoma (cHCC-CCA) sample.

148. The non-transitory computer-readable storage medium of any one of claims 142-144, wherein the cancer is a bile duct cancer.

149. The non-transitory computer-readable storage medium of claim 148, wherein the bile duct cancer is an intrahepatic bile duct cancer, an extrahepatic bile duct cancer, a perihilar bile duct cancer, or a distal bile duct cancer.

150. The non-transitory computer-readable storage medium of any one of claims 142-144, 148, and 149, wherein the cancer is a combined hepatocellular cholangiocarcinoma (cHCC-CCA).

151. The non-transitory computer-readable storage medium of any one of claims 142-150, wherein the genomic data for the test sample, the HCC genomic data, and the CCA genomic data each comprise a tumor purity.

152. The non-transitory computer-readable storage medium of any one of claims 142-151, wherein the genomic data for the test sample, the HCC genomic data, and the CCA genomic data each comprises a chromosomal aneuploidy status for one or more chromosomes or chromosome arms.

153. The non-transitory computer-readable storage medium of claim 152, wherein the chromosomal aneuploidy status comprises a loss status or a gain status of one or more of a 1q arm, 2q arm, 5p arm, 6p arm, 6q arm, 7q arm, 8p arm, 8q arm, 10q arm, 17p arm, 17q arm, 18q arm, 20p arm, 20q arm, 21p arm, and 22q arm.

154. The non-transitory computer-readable storage medium of any one of claims 142-153, wherein the genomic data for the test sample, the HCC genomic data, and the CCA genomic data each comprise a cancer cell fraction (CCF) for one or more genes, wherein the CCF for the one or more genes is differentially represented in CCA and HCC.

155. The non-transitory computer-readable storage medium of claim 154, wherein the CCF for one or more genes differentially represented in CCA and HCC comprises a CCF of one or more of TP53, CTNNB1, TERT, IDH1, and BAP1.

156. The non-transitory computer-readable storage medium of any one of claims 142-155, wherein the test genomic data, the HCC genomic data, and the CCA genomic data each comprise a functional variant status for each of one or more genes.

157. The non-transitory computer-readable storage medium of claim 156, wherein the functional variant status is a presence or an absence of the functional variant for the gene.

158. The non-transitory computer-readable storage medium of claim 156 or 157, wherein the functional variant caused by a single nucleotide variant (SNV), a multiple nucleotide variant (MNV), a copy number alteration, an indel, or a rearrangement.

159. The non-transitory computer-readable storage medium of any one of claims 156-158, wherein the one or more genes comprises ARID1A, BAP1, BRAF, CCND1, CDKN2A, CDKN2B, CTNNB1, ERBB2, FGFR2, IDH1, KRAS, MTAP, PBRMI, PIK3CA, PTEN, MYC, RB1, SMAD4, or TERT.

160. The non-transitory computer-readable storage medium of any one of claims 156-159, wherein the one or more genes comprises ARIDIA, BAP1, CDKN2A, CDKN2B, CTNNB1, FGFR2, IDH1, KRAS, PBRM1, MYC, or TERT.

161. The non-transitory computer-readable storage medium of any one of claims 142-160, wherein the genomic data for the test sample, the HCC genomic data, and the CCA genomic data each comprises a tumor mutational burden (TMB).

162. The non-transitory computer-readable storage medium of claim 161, wherein the TMB is a continuous numeric feature.

163. The non-transitory computer-readable storage medium of claim 161, wherein the TMB is a categorical feature.

164. The non-transitory computer-readable storage medium of any one of claims 142-163, wherein the genomic data for the sample, the HCC genomic data, and the CCA genomic data each comprises a microsatellite instability (MSI) status.

165. The non-transitory computer-readable storage medium of claim 164, wherein the MSI status is a categorical feature.

166. The non-transitory computer-readable storage medium of any one of claims 142-165, wherein genomic data for the sample, the HCC genomic data, and the CCA genomic data each comprises a genome-wide loss of heterozygosity (gLOH) status.

167. The non-transitory computer-readable storage medium of claim 166, wherein the gLOH status is a continuous numeric feature.

168. The non-transitory computer-readable storage medium of claim 166, wherein the gLOH status is a categorical feature.

169. The non-transitory computer-readable storage medium of any one of claims 142-168, herein the test data, the HCC data, and the CCA data each comprises an ancestry status.

170. The non-transitory computer-readable storage medium of claim 169, wherein the ancestry status is a genomic ancestry status.

171. The non-transitory computer-readable storage medium of claim 170, wherein the genomic ancestry status is a categorical feature, wherein the categorical feature is at least one of African, Ad Mixed American, East Asian, European, or South Asian.

172. The non-transitory computer-readable storage medium of any one of claims 142-171, wherein the test data, the HCC data, and the CCA data each comprise a hepatitis B virus (HBV) status.

173. The non-transitory computer-readable storage medium of claim 172, wherein the HBV status is determined by detecting a presence or absence of genomic HBV DNA.

174. The non-transitory computer-readable storage medium of any one of claims 142-173, wherein the test data, the HCC data, and the CCA data each further comprises one or more clinicopathological features.

175. The non-transitory computer-readable storage medium of claim 174, wherein the one or more clinicopathological features comprises an age of the subject at the time the sample was obtained from the subject, a biological sex of the subject, a sample biopsy site, or a cancer metastasis status.

176. The non-transitory computer-readable storage medium of any one of claims 142-175, wherein the genomic data for the sample, the HCC genomic data, and the CCA genomic data are each determined from sequencing data.

177. The non-transitory computer-readable storage medium of claim 176, wherein the sequencing data is targeted sequencing data.

178. The non-transitory computer-readable storage medium of claim 177, wherein the targeted sequencing data is generated using a hybrid-capture method.

179. The non-transitory computer-readable storage medium of claim any one of claims 176-178, wherein the sequencing data is generated using massively parallel sequencing.

180. The non-transitory computer-readable storage medium of any one of claims 142-179, wherein the cHCC-CCA machine-learning model is a tree-based classification model.

181. The non-transitory computer-readable storage medium of any one of claims 142-180, wherein the cHCC-CCA machine-learning model is an ensemble model.

182. The non-transitory computer-readable storage medium of any one of claims 142-181, wherein the cHCC-CCA machine-learning model is a bootstrap aggregated model.

183. The non-transitory computer-readable storage medium of any one of claims 142-182, wherein the cHCC-CCA machine-learning model is a random-forest model.

184. The non-transitory computer-readable storage medium of any one of claims 142-179, wherein the cHCC-CCA machine-learning model is a linear classification model.

185. The non-transitory computer-readable storage medium of any one of claims 142-184, wherein the sample is a solid tissue biopsy sample.

186. The non-transitory computer-readable storage medium of claim 185, wherein the solid tissue biopsy sample is a formalin-fixed paraffin-embedded (FFPE) sample.

187. The non-transitory computer-readable storage medium of any one of claims 142-184, wherein the sample is a liquid biopsy sample comprising circulating tumor DNA (ctDNA).

188. The non-transitory computer-readable storage medium of any one of claims 142-184, wherein the sample is a liquid biopsy sample comprising circulating tumor cells (CTCs).

189. The non-transitory computer-readable storage medium of claim 187 or 188, wherein the sample is a liquid biopsy sample and comprises blood, plasma, cerebrospinal fluid, sputum, stool, urine, or saliva.

190. The non-transitory computer-readable storage medium of any one of claims 148-189, wherein the one or more programs further include instructions, which when executed by one or more processors of an electronic device, cause the electronic device to generate a report identifying the cancer test sample as HCC-like. CCA-like, or ambiguous.

191. The non-transitory computer-readable storage medium of claim 190, wherein the one or more programs further include instructions, which when executed by one or more processors of an electronic device, cause the electronic device to display the report on an electronic display.

192. The non-transitory computer-readable storage medium of claim 190 or 191, wherein the one or more programs further include instructions, which when executed by one or more processors of an electronic device, cause the electronic device to transmit the report to the subject or a healthcare provider for the subject.

193. The non-transitory computer-readable storage medium of claim 192, wherein the report is transmitted via a computer network or a peer-to-peer connection.

194. The non-transitory computer-readable storage medium of claim 192 or 193, wherein the report is an electronic medical record.

195. A method comprising:
generating genomic data for a sample from a subject having cancer, comprising:
providing a plurality of nucleic acid molecules obtained from the sample from a subject;
ligating one or more adapters to one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
analyzing, by one or more processors, the plurality of sequence reads to generate the genomic data for the sample;
receiving, at at least one of the one or more processors, test data for the sample, wherein the test data comprises the genomic data;
inputting, using the at least one processor, the test data into a machine-learning model trained using a first carcinoma data comprising a first carcinoma genomic data from a plurality of first carcinoma samples and a second carcinoma data comprising second carcinoma genomic data from a plurality of second carcinoma samples, wherein the first carcinoma samples are different from the second carcinoma samples, and wherein the machine-learning model is configured to classify the sample, based on the test data, as first-carcinoma-like, second-carcinoma-like, or ambiguous; and
classifying, by the at least one processor using the machine-learning model, the sample as first-carcinoma-like, second-carcinoma-like, or ambiguous.

196. A method, comprising:
receiving, at one or more processors, test data for a sample from a subject with cancer, wherein the test data comprises genomic data for the sample;
inputting, using the at least one processor, the test data into a machine-learning model trained using a first carcinoma data comprising a first carcinoma genomic data from a plurality of first carcinoma samples and a second carcinoma data comprising second carcinoma genomic data from a plurality of second carcinoma samples, wherein the first carcinoma samples are different from the second carcinoma samples, and wherein the machine-learning model is configured to classify the sample, based on the test data, as first-carcinoma-like, second-carcinoma-like, or ambiguous; and
classifying, by the at least one processor using the machine-learning model, the sample as first-carcinoma-like, second-carcinoma-like, or ambiguous.
